(19) United States
(12) Patent Application Publication
(10) Pub. No.: US 2025/0040944 A1
(43) Pub. Date: Feb. 6, 2025

(54) FEMORAL FIXATION DEVICES, SYSTEMS, AND METHODS

(71) Applicant: RTG Scientific, LLC, Austin, TX (US)

(72) Inventors: Raymond WHITE, Windhamm, ME (US); Matthew CAMUSO, Falmouth, ME (US); Timothy WEBER, Austin, TX (US); Michael PARKER, Austin, TX (US); Corey JOHNSON, Rochester, MN (US); Bruce HOFFMAN, Smithfield, UT (US); Spencer BUNN, Nibley, UT (US); Andrew FAUTH, North Logan, UT (US)

(21) Appl. No.: 18/923,595

(22) Filed: Oct. 22, 2024

Related U.S. Application Data
(63) Continuation-in-part of application No. 17/468,806, filed on Sep. 8, 2021, now Pat. No. 12,121,271.
(60) Provisional application No. 63/147,640, filed on Feb. 9, 2021.

(51) Int. Cl. A61B 17/17; A61B 17/56; A61B 17/74

(52) U.S. Cl. CPC A61B 17/1721; A61B 17/74; A61B 2017/564

(57) ABSTRACT

Surgical kits for implanting a femoral fastener in a femoral bone may include the femoral fastener and at least one of: a targeting guide assembly, an anti-rotation assembly, and a plurality of tap tools. The targeting guide assembly may include a targeting guide base having a locating slot and a removal slot that are formed therethrough. The removal slot may extend from an inferior end of the targeting guide base into the locating slot. A guide pin may be placed through the locating slot and into the neck and head of the femoral bone to guide placement of the femoral fastener therein. The targeting guide base may be movable in a superior direction to release the guide pin inferiorly through the removal slot that extends from the locating slot to the inferior end of the targeting guide base.

### CROSS-REFERENCE TO RELATED APPLICATIONS

This application is a continuation-in-part of U.S. patent application Ser. No. 17/468,806 filed on Sep. 8, 2021, entitled “FEMORAL FIXATION DEVICES, SYSTEMS, AND METHODS”, which claims the benefit of U.S. Provisional Patent Application Ser. No. 63/147,640 filed on Feb. 9, 2021, entitled “FASTENING DEVICES, SYSTEMS, AND METHODS”.

The foregoing are incorporated by reference as though set forth herein in their entirety.

### TECHNICAL FIELD

The present disclosure relates to fixation devices, systems, instruments, and methods. More specifically, the present disclosure relates to femoral fixation devices, systems, instruments, and methods for improved fixation.

### BACKGROUND

Surgical procedures involving femoral fasteners implanted in bone and other tissues can become lose over time due to multi-axial forces and off-axis loading scenarios that may be applied to the femoral fastener during the healing process. Traditional femoral fastener thread designs may not provide sufficient fixation to overcome these multi-axial forces and off-axis loading scenarios.

Accordingly, femoral fastener devices, systems, instruments, and methods with improved thread designs that increase bone fixation and load sharing between a bone/fastener interface experiencing multi-axial and off-loading conditions would be desirable.

### SUMMARY

The various femoral fastening devices, systems, and methods of the present disclosure have been developed in response to the present state of the art, and in particular, in response to the problems and needs in the art that have not yet been fully solved by currently available femoral fastening devices, systems, instruments, and methods. In some embodiments, the femoral fastening devices, systems, instruments, and methods of the present disclosure may provide improved fixation and load sharing between a bone/fastener interface under multi-axial and off-loading conditions, as well as improved instruments and methods for implanting the fastener in a bone.

In some embodiments, a surgical kit for implanting a femoral fastener in a femoral bone may include a femoral fastener and a targeting guide assembly. The femoral fastener may include a shaft having a longitudinal axis, a proximal end, a distal end, a longitudinal passageway formed through the shaft, and a helical thread disposed about the shaft between a first location and a second location along the shaft. The helical thread may include at least one undercut surface such that, when the femoral fastener is implanted within a neck and a head of a femoral bone: at least a portion of the helical thread may reside within the head of the femoral bone; the at least one undercut surface of the helical thread may be angled toward one of the proximal end and the distal end of the femoral fastener; and the at least one undercut surface of the helical thread may be configured to transmit at least one force from the head of the femoral bone to the neck of the femoral bone. The targeting guide assembly may include a targeting guide base, and a targeting guide insert. The targeting guide base may include a superior end, an inferior end a bone-facing surface, an opposing surface opposite the bone-facing surface, a locating slot formed through the targeting guide base between the bone-facing surface and the opposing surface, and a removal slot formed through the targeting guide base between the bone-facing surface and the opposing surface that extends from the inferior end of the targeting guide base to the locating slot. The targeting guide insert may include a proximal end, a distal end, at least one insert passageway formed through the targeting guide insert between the proximal end and the distal end of the targeting guide insert, and an insert base connection feature at the distal end of the targeting guide insert. The bone-facing surface of the targeting guide base may be configured to engage a lateral surface of the femoral bone. The insert base connection feature may be configured to removably couple the targeting guide insert to the targeting guide base at the locating slot. The at least one insert passageway of the targeting guide insert may be configured to orient a guide pin placed therethrough into the neck and head of the femoral bone to guide placement of the femoral fastener therein. Once the guide pin has been placed into the neck and the head of the femoral bone, the targeting guide insert may be configured to detach from the targeting guide base and slidingly disengage from the guide pin along a proximal direction. The targeting guide base may also be movable in a superior direction to release the guide pin inferiorly through the removal slot that extends from the locating slot to the inferior end of the targeting guide base.

In some embodiments of the surgical kit, the at least one insert passageway may comprise at least one of: a cylindrical shape, a conical shape, an oval shape, a flared shape, and a tapered shape.

In some embodiments of the surgical kit, the at least one insert passageway may include a plurality of insert passageways formed through the targeting guide insert.

In some embodiments of the surgical kit, the insert base connection feature may include an insert projection configured to be at least partially received within the locating slot of the targeting guide base.

In some embodiments, the surgical kit may also include a targeting guide positioner. The targeting guide positioner may include a positioner shaft, a positioner first arm projecting from a distal end of the positioner shaft, a positioner second arm projecting from the distal end of the positioner shaft opposite the positioner first arm, at least one positioner base connection feature configured to couple the targeting guide positioner to the targeting guide base, and at least one positioner insert connection feature configured to couple the targeting guide positioner to the targeting guide insert.

In some embodiments of the surgical kit, the targeting guide base may also include one or more placement pin holes configured to receive one or more placement pins therethrough to couple the targeting guide base to the femoral bone.

In some embodiments of the surgical kit, the targeting guide base may also include one or more bone fixation members configured to stabilize the targeting guide base with respect to the femoral bone.

In some embodiments, a surgical kit for implanting a femoral fastener in a femoral bone may include a femoral fastener and an anti-rotation assembly. The femoral fastener may include a shaft having a longitudinal axis, a proximal end, a distal end, a longitudinal passageway formed through the shaft, and a helical thread disposed about the shaft between a first location and a second location along the shaft. The helical thread may include at least one undercut surface such that, when the femoral fastener is implanted within a neck and a head of a femoral bone: at least a portion of the helical thread may reside within the head of the femoral bone; the at least one undercut surface of the helical thread may be angled toward one of the proximal end and the distal end of the femoral fastener; and the at least one undercut surface of the helical thread may be configured to transmit at least one force from the head of the femoral bone to the neck of the femoral bone. The anti-rotation assembly may include at least one anti-rotation placement pin and an anti-rotation sleeve having a proximal end, a distal end, an anti-rotation sleeve passageway extending between the proximal end and the distal end of the anti-rotation sleeve defining an inner surface therein, and at least one anti-rotation pin hole disposed about the inner surface of the anti-rotation sleeve that is configured to receive the at least one anti-rotation placement pin therethrough. When the anti-rotation assembly is coupled to the femoral bone: the distal end of the anti-rotation sleeve is positioned adjacent a lateral surface of the femoral bone; the at least one anti-rotation placement pin is inserted through the at least one anti-rotation pin hole and into the neck and the head of the femoral bone to stabilize a bone fracture formed between the neck and the head of the femoral bone; the anti-rotation sleeve passageway is configured to receive at least one rotatable tool therethrough that imparts at least one torque force on the head of the femoral bone relative to the neck of the femoral bone across the bone fracture; and the anti-rotation assembly is configured to resist the at least one torque force imparted on the head of the femur to stabilize the bone fracture during rotation of the at least one rotatable tool.

In some embodiments of the surgical kit, the at least one anti-rotation placement pin may include a plurality of anti-rotation placement pins, and the at least one anti-rotation pin hole may include a plurality of anti-rotation pin holes disposed about the inner surface of the anti-rotation sleeve and configured to receive the plurality of anti-rotation placement pins therethrough.

In some embodiments of the surgical kit, the distal end of the anti-rotation sleeve may be angled.

In some embodiments of the surgical kit, the distal end of the anti-rotation sleeve may be straight.

In some embodiments of the surgical kit, the anti-rotation sleeve may also include at least one anti-rotation pin hole projection, and the at least one anti-rotation pin hole may be formed through the at least one anti-rotation pin hole projection.

In some embodiments, the surgical kit may also include the at least one rotatable tool, which may comprise at least one of: a reamer tool, a drill tool, a tap tool, and an inserter tool.

In some embodiments, the surgical kit may also include at least one centering sleeve receivable within the anti-rotation sleeve passageway. The at least one centering sleeve may include at least one of: a tool centering sleeve, a guide wire centering sleeve, and a femoral fastener centering sleeve.

In some embodiments of the surgical kit, a surgical kit for implanting a femoral fastener in a femoral bone may include a femoral fastener, a first tap tool, and a second tap tool. The femoral fastener may include a shaft having a longitudinal axis, a proximal end, a distal end, a longitudinal passageway formed through the shaft, and a helical thread disposed about the shaft between a first location and a second location along the shaft. The helical thread may include at least one undercut surface such that, when the femoral fastener is implanted within a neck and a head of a femoral bone: at least a portion of the helical thread may reside within the head of the femoral bone; the at least one undercut surface of the helical thread may be angled toward one of the proximal end and the distal end of the femoral fastener; and the at least one undercut surface of the helical thread may be configured to transmit at least one force from the head of the femoral bone to the neck of the femoral bone across a bone fracture that is formed therebetween. The first tap tool may include first cutting teeth having a first height, and the second tap tool may include second cutting teeth having a second height. The first height of the first cutting teeth may be less than the second height of the second cutting teeth. The first tap tool may be configured to cut a first tapped bone thread in the head of the femoral bone at the first height of the first cutting teeth, thereby imparting a first torque force on the head relative to the neck across the bone fracture as the first tap tool rotates. The second taptool may be configured to follow the first tapped bone thread and cut a remaining height of the first tapped bone thread at the second height of the second cutting teeth, thereby imparting a second torque force on the head relative to the neck across the bone fracture as the second tap tool rotates. In this manner, the second torque force imparted on the head may be reduced by cutting the first tapped bone thread in the head at the first height before cutting the remaining height of the first tapped bone thread at the second height in order to reduce the torque forces that are applied to the head, thereby maintaining stabilization of the bone fracture as the tap tools rotate.

In some embodiments, the surgical kit may also include one or more tap tools (or a plurality of tap tools) having one or more cutting teeth heights (or a plurality of cutting teeth heights) that are intermediate the first height and the second height in order to further reduce the torque forces applied to the head by successively cutting the first tapped bone thread into the head, thereby maintaining the stabilization of the bone fracture as the tap tools rotate.

In some embodiments of the surgical kit, the plurality of cutting teeth heights may be configured to increase in a linear fashion between the first height and the second height.

In some embodiments of the surgical kit, the plurality of cutting teeth heights may be configured to increase in a non-linear fashion between the first height and the second height.

In some embodiments of the surgical kit, at least one of: the first tap tool may include at least one first cutting flute configured to move bone chips in a proximal direction along the first tap tool; and the second tap tool may include at least one second cutting flute configured to move bone chips in the proximal direction along the second tap tool.

In some embodiments of the surgical kit, at least one of: the first tap tool may include a first tap tool torque connection feature configured to receive torque forces to rotate the first tap tool; and the second tap tool may include a second tap tool torque connection feature configured to receive torque forces to rotate the second tap tool.

These and other features and advantages of the present disclosure will become more fully apparent from the following description and appended claims, or may be learned by the practice of the devices, systems, methods, and instruments set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1A illustrates a front perspective view of a fastener, according to an embodiment of the present disclosure;

FIG. 1B illustrates a rear perspective view of the fastener of FIG. 1A;

FIG. 1C illustrates a side view of the fastener of FIG. 1A;

FIG. 1D illustrates a cross-sectional side view of the fastener of FIG. 1C, taken along the line A-A;

FIG. 2 illustrates a partial cross-sectional side view of a fastener comprising crescent-shaped threading;

FIG. 3A illustrates a rear perspective view of a femoral fastener, according to an embodiment of the present disclosure;

FIG. 3B illustrates a front perspective view of the femoral fastener of FIG. 3A;

FIG. 3C illustrates a side view of the femoral fastener of FIG. 3A;

FIG. 3D illustrates a cross-sectional side view of the fastener shown in FIG. 3C, taken along the line B-B;

FIG. 4A illustrates a perspective side view of a femoral support member, according to an embodiment of the present disclosure;

FIG. 4B illustrates another perspective side view of the femoral support member of FIG. 4A;

FIG. 4C illustrates a front view of the femoral support member of FIG. 4A;

FIG. 4D illustrates a rear view of the femoral support member of FIG. 4A;

FIG. 5A illustrates a front view of a stop member, according to an embodiment of the present disclosure;

FIG. 5B illustrates a rear perspective view of the stop member of FIG. 5A;

FIG. 5C illustrates a front perspective view of the stop member of FIG. 5A;

FIG. 5D illustrates a side view of the stop member of FIG. 5A;

FIG. 5E illustrates a side view of the stop member of FIG. 5A with a stop member projection;

FIG. 5F illustrates a side view of the stop member of FIG. 5A with another stop member projection;

FIG. 6 illustrates an exploded view of a femoral fixation assembly, according to an embodiment of the present disclosure;

FIG. 7 illustrates a side view of the femoral fixation assembly of FIG. 6, after it has been assembled;

FIG. 8 illustrates a side view of the femoral fixation assembly of FIG. 6 implanted in a femur;

FIG. 9 illustrates a front view of the femoral fixation assembly and femur shown in FIG. 8;

FIG. 10 illustrates a cross-sectional side view of the femoral fixation assembly and femur shown in FIG. 9, taken along the line C-C;

FIG. 11 illustrates a perspective side view of a targeting guide assembly placed adjacent a femur, according to an embodiment of the present disclosure;

FIG. 12 illustrates a cross-sectional side view of a targeting guide insert, according to an embodiment of the present disclosure;

FIG. 13 illustrates a side view of a guide pin inserted into a femur;

FIG. 14 illustrates a side view of a guide pin depth gauge adjacent to a femur, according to an embodiment of the present disclosure;

FIG. 15 illustrates a side view of a tissue shield and reamer adjacent a femur, according to embodiments of the present disclosure;

FIG. 16 illustrates a side view of drill bit, adjustable depth stop, and centering sleeve, according to embodiments of the present disclosure;

FIG. 17 illustrates a side view of a tap device, according to an embodiment of the present disclosure;

FIG. 18A illustrates a close up partial view of a connection feature between an inserter and a femoral fastener prior to engagement, according to an embodiment of the present disclosure;

FIG. 18B illustrates a side view of the connection feature of FIG. 18A after engagement;

FIG. 18C illustrates a side view of the connection feature of FIG. 18A showing an inserter coupler that is inside the inserter before it engages with an internal thread of the femoral fastener;

FIG. 18D illustrates a side view of the connection feature of FIG. 18C after the inserter coupler has been engaged with the internal thread of the femoral fastener;

FIG. 19 illustrates a side view of a centering sleeve and inserter facilitating placement of a femoral fastener within a femur;

FIG. 20 illustrates a side view of the inserter of FIG. 19 being utilized to couple a femoral support member to the femoral fastener;

FIG. 21 illustrates a side view of the femoral support member and femoral fastener after the inserter has been removed;

FIG. 22 illustrates a side view of an impactor and mallet utilized to seat the femoral support member against the femur;

FIG. 23A illustrates a side view of a compression screw inserted into the femoral support member and the femoral fastener;

FIG. 23B illustrates a close-up view of the compression screw shown in FIG. 23A;

FIG. 24 illustrates a side view of a drill bit and drill bit guide forming bone tunnels in the femur, according to an embodiment of the present disclosure;

FIG. 25 illustrates a side view of a bone tunnel depth gauge utilized to measure a depth of a bone tunnel, according to an embodiment of the present disclosure;

FIG. 26 illustrates a side view of a driver installing one or more bone plate fasteners into the femur, according to an embodiment of the present disclosure;

FIG. 27 illustrates a side view of a drill bit and drill bit guide forming bone tunnels into the head of the femur, according to another embodiment of the present disclosure;

FIG. 28 illustrates a side view of the driver installing one or more support fasteners into the head of the femur;

FIG. 29 illustrates a side view of the femoral fixation assembly of FIG. 28 after the one or more support fasteners have been installed;

FIG. 30 illustrates a side view of an extension plate being coupled to the femoral support member, according to an embodiment of the present disclosure;

FIG. 31 illustrates a side view of the femoral fixation assembly of FIG. 30 after the extension plate has been coupled to the femoral support member and additional support fasteners have been inserted into the head of the femur;

FIG. 32 illustrates a side view of the femoral fixation assembly of FIG. 29 after a trochanter plate has been coupled to the femoral support member and additional support fasteners have been inserted into the head of the femur, according to another embodiment of the present disclosure;

FIG. 33 illustrates a side view of the femoral fixation assembly of FIG. 32 showing a drill bit forming one or more bone tunnels to receive one or more trochanter plate fasteners to secure the trochanter plate to the femur;

FIG. 34A illustrates a perspective side view of the femoral fixation assembly shown in FIG. 33 after the one or more trochanter plate fasteners have been installed;

FIG. 34B illustrates a perspective front view of the femoral fixation assembly shown in FIG. 34A;

FIG. 35 illustrates a perspective front view of a targeting guide base, according to another embodiment of the present disclosure;

FIG. 36A illustrates a perspective side view of a femoral support member, according to another embodiment of the present disclosure;

FIG. 36B illustrates another perspective side view of the femoral support member of FIG. 36A;

FIG. 36C illustrates a front view of the femoral support member of FIG. 36A;

FIG. 36D illustrates a rear view of the femoral support member of FIG. 36A;

FIG. 37A illustrates a perspective side view of a femoral support member, according to another embodiment of the present disclosure;

FIG. 37B illustrates another perspective side view of the femoral support member of FIG. 37A;

FIG. 37C illustrates a front view of the femoral support member of FIG. 37A;

FIG. 37D illustrates a rear view of the femoral support member of FIG. 37A;

FIG. 38 illustrates a side view of a femoral fixation assembly utilizing the femoral support member of FIG. 37A and the femoral fastener of FIG. 3A;

FIG. 39 illustrates a side view of the femoral fixation assembly of FIG. 38 implanted in a femur;

FIG. 40 illustrates a front view of the femoral fixation assembly and femur shown in FIG. 39;

FIG. 41A illustrates a front view of a targeting guide base, according to an embodiment of the present disclosure;

FIG. 41B illustrates a rear view of the targeting guide base of FIG. 41A;

FIG. 41C illustrates an inferior end view of the targeting guide base of FIG. 41A;

FIG. 41D illustrates a superior end view of the targeting guide base of FIG. 41A;

FIG. 41E illustrates a side view of the targeting guide base of FIG. 41A;

FIG. 42A illustrates a perspective top view of a targeting guide insert 840, according to an embodiment of the present disclosure;

FIG. 42B illustrates a perspective bottom view of the targeting guide insert 840 of FIG. 42A;

FIG. 42C illustrates a perspective proximal end view of the targeting guide insert 840 of FIG. 42A;

FIG. 42D illustrates a perspective distal end view of the targeting guide insert 840 of FIG. 42A;

FIG. 43 illustrates a perspective distal end view of a targeting guide positioner, according to an embodiment of the present disclosure;

FIG. 44A illustrates a perspective distal end view of a handle, according to an embodiment of the present disclosure;

FIG. 44B illustrates a perspective proximal end view of the handle of FIG. 44A;

FIG. 45 illustrates an exploded view of a targeting guide assembly, according to an embodiment of the present disclosure;

FIG. 46 illustrates a perspective view of the targeting guide assembly of FIG. 45 assembled on a femur;

FIG. 47 illustrates a perspective view of the targeting guide assembly of FIG. 45 with placement pins;

FIG. 48A illustrates a perspective view of the targeting guide assembly of FIG. 46 with a guide pin placed therethrough;

FIG. 48B illustrates another perspective view of the targeting guide assembly of FIG. 48A showing the guide pin placed within the head of the femur;

FIG. 49 illustrates a side view of the guide pin depth gauge placed adjacent the femur;

FIG. 50 illustrates a side view of the tissue shield and reamer placed adjacent the femur;

FIG. 51A illustrates a perspective proximal end view of an anti-rotation sleeve, according to an embodiment of the present disclosure;

FIG. 51B illustrates a perspective distal end view of the anti-rotation sleeve of FIG. 51A;

FIG. 52A illustrates a perspective distal end view of an anti-rotation sleeve, according to another embodiment of the present disclosure;

FIG. 52B illustrates a side view of the anti-rotation sleeve of FIG. 52A;

FIG. 52C illustrates a bottom view of the anti-rotation sleeve of FIG. 52A;

FIG. 53 illustrates a distal end view of the anti-rotation sleeve of FIG. 51A;

FIG. 54 illustrates a distal end view of an anti-rotation sleeve, according to another embodiment of the present disclosure;

FIG. 55 illustrates a distal end view of an anti-rotation sleeve, according to another embodiment of the present disclosure;

FIG. 56A illustrates a perspective distal end view of a guide wire centering sleeve, according to an embodiment of the present disclosure;

FIG. 56B illustrates a perspective proximal end view of the guide wire centering sleeve of FIG. 56A;

FIG. 57A illustrates a perspective distal end view of a tool centering sleeve, according to an embodiment of the present disclosure;

FIG. 57B illustrates a perspective proximal end view of the tool centering sleeve of FIG. 57A;

FIG. 58 illustrates a perspective view of an anti-rotation assembly coupled to a femur, according to an embodiment of the present disclosure;

FIG. 59 illustrates a perspective view of another anti-rotation assembly coupled to a femur, according to another embodiment of the present disclosure;

FIG. 60 illustrates a perspective view of the anti-rotation assembly of FIG. 59 with the tool centering sleeve placed therein;

FIG. 61 illustrates a perspective view of the anti-rotation assembly of FIG. 60 with the guide wire centering sleeve placed therein;

FIG. 62A illustrates a perspective view of the anti-rotation assembly of FIG. 61 with the guide pin placed therethrough;

FIG. 62B illustrates another perspective view of the anti-rotation assembly of FIG. 62A showing the anti-rotation placement pins and the guide pin placed within the head of the femur;

FIG. 63A illustrates a perspective view of the anti-rotation assembly of FIG. 62A with the guide wire centering sleeve removed and a drill tool placed therein;

FIG. 63B illustrates another perspective view of the anti-rotation assembly of FIG. 63A showing the anti-rotation placement pins and the drill bit of the drill tool placed within the head of the femur;

FIG. 64A illustrates a perspective distal end view of a tap tool, according to embodiments of the present disclosure;

FIG. 64B illustrates a close up distal end view of a cutting head of the tap tool of FIG. 64A;

FIG. 65A illustrates a perspective view of the anti-rotation assembly of FIG. 63B with the drill tool removed and the tap tool placed therein;

FIG. 65B illustrates another perspective view of the anti-rotation assembly of FIG. 65A showing the anti-rotation placement pins and the tap tool placed within the head of the femur;

FIG. 66 illustrates a perspective view of the anti-rotation assembly of FIG. 65A with the tap tool removed and a femoral fastener centering sleeve inserted therein to facilitate insertion of the femoral fastener into the head of the femur;

FIG. 67 illustrates a perspective view of the anti-rotation assembly of FIG. 66 with the inserter tool placed therein to rotatably insert the femoral fastener into the head of the femur;

FIG. 68 illustrates a perspective view of the impactor and mallet seating the femoral support member against the femur;

FIG. 69 illustrates a perspective view of the femoral fixation assembly installed in the femur; and

FIG. 70 illustrates another perspective view of FIG. 69 showing the femoral fastener, support fasteners placed within the head of the femur, and bone plate fasteners placed within the shaft of the femur.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure, but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means outboard deviation of the knees (away from the sagittal plane) from the line between the hip and ankle, resulting in a “bowlegged” stance. Valgus means inboard deviation of the knees (toward the sagittal plane) from the line between the hip and ankle, resulting in a “knock-kneed” stance.

Although the present disclosure illustrates bone fixation concepts with reference to a femoral bone, it will also be understood that the devices, systems, instruments, and methods disclosed herein may be adapted for use within any bone.

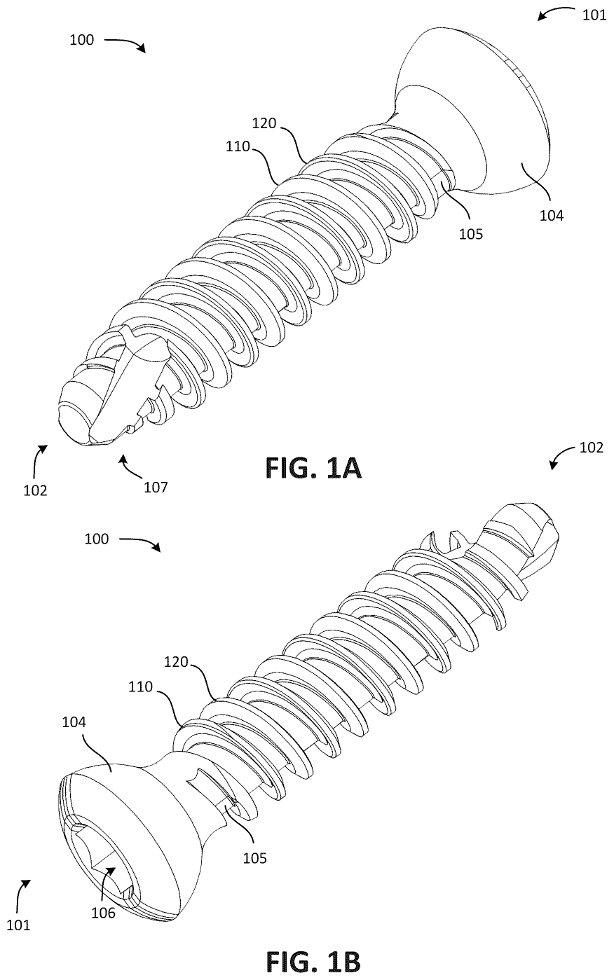

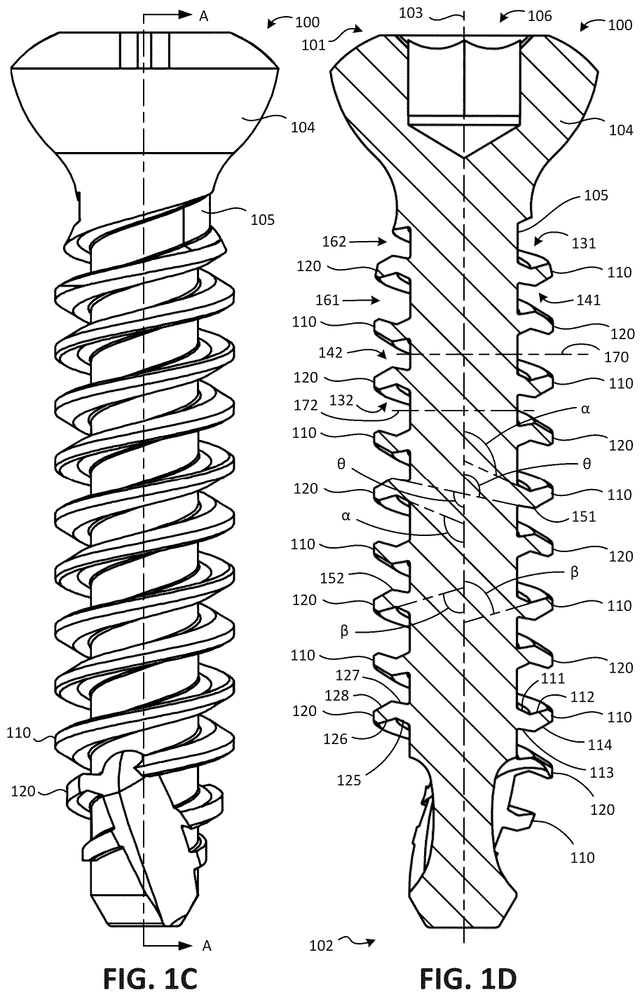

FIGS. 1A-D illustrate various views of a fastener 100, implantable bone anchor, or bone screw, according to an example of the present disclosure. Specifically, FIG. 1A is a front perspective view of the fastener 100, FIG. 1B is a rear perspective view of the fastener 100, FIG. 1C is a side view of the fastener 100, and FIG. 1D is a cross-sectional side view of the fastener 100 taken along the line A-A in FIG. 1C.

In general, the fastener 100 may include a shaft 105 having a proximal end 101, a distal end 102, and a longitudinal axis 103. The fastener 100 may also include a head 104 located at the proximal end 101 of the shaft 105, a torque connection interface 106 formed in/on the head 104 (in either a male/female configuration), and a self-tapping feature 107 formed in the shaft 105, such as the distal end 102 of the shaft 105, etc.

In some embodiments, the fastener 100 may include a first helical thread 110 disposed about the shaft 105, and a second helical thread 120 disposed about the shaft 105 adjacent the first helical thread 110.

In some embodiments, the fastener 100 may include a “dual start” or “dual lead” thread configuration comprising the first helical thread 110 and the second helical thread 120.

In some embodiments, a depth of the first helical thread 110 and/or the second helical thread 120 with respect to the shaft 105 may define a major diameter vs. a minor diameter of the shaft 105 alone.

In some embodiments, a major diameter and/or a minor diameter of the fastener 100 may be constant or substantially constant along the entire length of the fastener, along a majority of the length of the fastener, or along any length of the fastener 100. In these embodiments, a constant minor diameter may help avoid blowout of narrow/delicate bones (e.g., a pedicle or other bones) when inserting a fastener into a bone. In some embodiments, a pilot hole may first be drilled into a narrow/delicate bone and then a fastener having a similar minor diameter in comparison to the diameter of the pilot hole may be chosen to avoid blowout when inserting the fastener into the bone.

In some embodiments, a depth of the first helical thread 110 and/or the second helical thread 120 with respect to the shaft 105 may vary along a length of the shaft 105 to define one or more major diameters of the fastener 100 and/or one or more regions along the fastener 100 may comprise a one or more continuously variable major diameters.

In some embodiments, a thickness of the shaft 105 may vary along a length of the shaft 105 to define one or more minor diameters of the fastener 100, and/or one or more regions along the fastener 100 may comprise one or more continuously variable minor diameters. In some embodiments, a thickness/height/width/length/pitch/angle/shape, etc., of the first helical thread 110 and/or the second helical thread 120 (or any additional helical thread) may vary along a length of the shaft 105. For example, a thickness/height/width/length/pitch/angle/shape, etc., of the first helical thread 110 and/or the second helical thread 120 may be greater towards the tip of the fastener and thinner towards the head of the fastener (or vice versa) in either a discrete or continuously variable fashion, etc., or combinations thereof.

In some embodiments, the major and/or minor diameters may increase toward a proximal end or head of a fastener (or vice versa) in order to increase bone compaction as the fastener is terminally inserted into the bone/tissue.

In some embodiments, a pitch of the first helical thread 110 and/or the second helical thread 120 may vary along a length of the fastener 100.

In some embodiments, the fastener 100 may include a plurality of helical threads disposed about the shaft 105. However, it will also be understood that any of the fasteners disclosed or contemplated herein may include a single helical thread disposed about the shaft of the fastener. Moreover, the fastener 100 may comprise a nested plurality of helical threads having different lengths (not shown). As one non-limiting example, the fastener 100 may include a first helical thread 110 that is longer than a second helical thread 120, such that the fastener 100 comprises dual threading along a first portion of the shaft 105 and single threading along a second portion of the shaft 105.

In some embodiments, the plurality of helical threads may include three helical threads comprising a “triple start” or “triple lead” thread configuration (not shown).

In some embodiments, the plurality of helical threads may include four helical threads comprising a “quadruple start” or “quadruple lead” thread configuration (not shown).

In some embodiments, the plurality of helical threads may include more than four helical threads (not shown).

In some embodiments, the fastener 100 may include first threading with any of the shapes disclosed herein oriented toward one of the proximal end and the distal end of the fastener 100, with the first threading located proximate the distal end of the fastener 100, as well as second threading with any of the shapes disclosed herein oriented toward the other one of the proximal end and the distal end of the fastener 100, with the second threading located proximate the head of the fastener 100 (not shown).

In some embodiments, the fastener 100 may include multiple threading (e.g., dual helical threading, etc.) with any of the shapes disclosed herein located proximate one of the proximal end and the distal end of the fastener 100, as well as single threading with any of the shapes disclosed herein with the second threading located proximate the other of the proximal end and the distal end of the fastener 100.

In some embodiments, the first helical thread 110 may include a plurality of first concave undercut surfaces 131 and a plurality of first convex undercut surfaces 141.

In some embodiments, the second helical thread 120 may include a plurality of second concave undercut surfaces 132 and a plurality of second convex undercut surfaces 142.

In some embodiments, when the fastener 100 is viewed in section along a plane that intersects the longitudinal axis 103 of the shaft 105 (e.g., see FIG. 1D), the plurality of first concave undercut surfaces 131 and the plurality of second convex undercut surfaces 142 may be oriented toward (i.e., point toward) the proximal end 101 of the shaft 105.

In some embodiments, the plurality of first convex undercut surfaces 141 and the plurality of second concave undercut surfaces 132 may be oriented toward (i.e., point toward) the distal end 102 of the shaft 105.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one substantially flat surface.

In some embodiments, when the fastener 100 is viewed in section along a plane intersecting the longitudinal axis 103 of the shaft 105, the first helical thread 110 may comprise a plurality of first bent shapes (comprising at least one surface that is angled relative to the longitudinal axis 103 of the shaft 105 and/or at least one undercut surface) with a plurality of first intermediate portions 151 that are oriented toward (i.e., point toward) the distal end 102 of the shaft 105. This may be referred to as “standard” threading, having a “standard” orientation.

In some embodiments, when the fastener 100 is viewed in section along a plane intersecting the longitudinal axis 103 of the shaft 105, the second helical thread 120 may comprise a plurality of second bent shapes (comprising at least one surface that is angled relative to the longitudinal axis 103 of the shaft 105 and/or at least one undercut surface) with a plurality of second intermediate portions 152 that are oriented toward (i.e., point toward) the proximal end 101 of the shaft 105. This may be referred to as “inverted” threading, having an “inverted” orientation.

In some embodiments, one or more helical threads may morph/transition between a standard orientation and an inverted orientation along a shaft of a fastener.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one curved surface.

As shown in FIG. 1D, the proximally-oriented and distally-oriented surfaces of the first helical thread 110 (i.e., the first concave undercut surfaces 131 and the first convex undercut surfaces 141 in the fastener 100 of FIG. 1D) may not have mirror symmetry relative to each other about any plane perpendicular to the longitudinal axis 103 of the fastener 100. Rather, the first concave undercut surfaces 131 and the first convex undercut surfaces 141 may be generally parallel to each other. The same may be true for the second helical thread 120, in which the second concave undercut surfaces 132 and the second convex undercut surfaces 142 may not have mirror symmetry relative to each other, but may be generally parallel to each other.

Conversely, as also shown in FIG. 1D, the proximally-oriented surfaces of the first helical thread 110 may have mirror symmetry relative to the distally-oriented surfaces of the second helical thread 120. Specifically, the first concave undercut surfaces 131 may have mirror symmetry relative to the second convex undercut surfaces 142 about a plane 170 that bisects the space between them, and lies perpendicular to the longitudinal axis 103.

Similarly, the distally-oriented surfaces of the first helical thread 110 may have mirror symmetry relative to the proximally-oriented surfaces of the second helical thread 120. Specifically, the second concave undercut surfaces 132 may have mirror symmetry relative to the first convex undercut surfaces 141 about a plane 172 that bisects the space between them, and lies perpendicular to the longitudinal axis 103.

This mirror symmetry may be present along most of the length of the first helical thread 110 and the second helical thread 120, with symmetry across different planes arranged between adjacent turns of the first helical thread 110 and the second helical thread 120 along the length of the longitudinal axis 103. Such mirror symmetry may help more effectively capture bone between the first helical thread 110 and the second helical thread 120, and may also facilitate manufacture of the fastener 100.

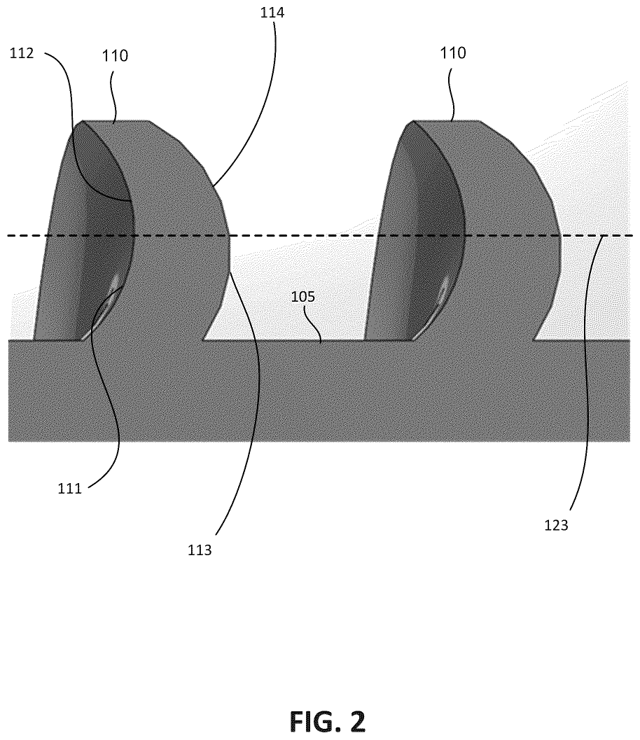

In some embodiments, when the fastener 100 is viewed in section along a plane intersecting the longitudinal axis 103 of the shaft 105, the first helical thread 110 may include at least one partial crescent shape that is oriented toward (i.e., points toward) the distal end 102 of the shaft 105 and/or the proximal end 101 of the shaft 105. FIG. 2 illustrates a partial cross-sectional view of a fastener comprising crescent shapes, as one non-limiting example of such an embodiment.

In some embodiments (not shown), when the fastener 100 is viewed in section along a plane intersecting the longitudinal axis 103 of the shaft 105, the first helical thread 110 may include at least one partial crescent shape that is oriented toward (i.e., points toward) the distal end 102 of the shaft 105, and the second helical thread 120 may include at least one partial crescent shape that is oriented toward (i.e., points toward) the proximal end 101 of the shaft 105.

In some embodiments (not shown), the first helical thread 110 may include a first plurality of partial crescent shapes that are oriented toward (i.e., point toward) the distal end 102 of the shaft 105, and the second helical thread 120 may include a second plurality of partial crescent shapes that are oriented toward (i.e., point toward) the proximal end 101 of the shaft 105.

In some embodiments (not shown), the first plurality of partial crescent shapes and the second plurality of partial crescent shapes may be arranged in alternating succession along the shaft 105 of the fastener 100.

In some embodiments, the first helical thread 110 may be bisected by the line 123 shown in FIG. 2 with each crescent shape including a plurality of first undercut surfaces 111, a plurality of second undercut surfaces 112, a plurality of third undercut surfaces 113, and a plurality of fourth open surfaces 114 similar to the helical threading shown in FIG. 1D, except with curved surfaces in place of flat surfaces.

In some embodiments, the plurality of first undercut surfaces 111 and the plurality of second undercut surfaces 112 may comprise concave curved surfaces. However, it will be understood that portions of the plurality of first undercut surfaces 111 and/or portions of the plurality of second undercut surfaces 112 may also comprise convex curved surfaces and/or flat surfaces (not shown in FIG. 2).

In some embodiments, the plurality of third undercut surfaces 113 and the plurality of fourth open surfaces 114 may comprise convex curved surfaces. However, it will be understood that portions of the plurality of third undercut surfaces 113 and the plurality of fourth open surfaces 114 may also comprise concave curved surfaces and/or flat surfaces (not shown in FIG. 2).

In some embodiments, the plurality of third undercut surfaces 113 and the plurality of fourth open surfaces 114 may be replaced by a ramped surface (such as that utilized in a standard buttress thread design) without any undercuts (not shown in FIG. 2). Likewise, any of the other thread designs disclosed herein may utilize a ramped or buttress thread design on at least one side of the helical thread.

In some embodiments, a fastener may have only standard threads or only inverted threads. The type of threads that are desired may depend on the type and/or magnitude of loads to be applied to the fastener. For example, a fastener loaded axially away from the bone in which it is implanted may advantageously have a standard thread, while a fastener loaded axially toward the bone in which it is implanted may advantageously have an inverted thread. A fastener that may experience multi-axial loading and/or off-loading conditions may advantageously include at least one standard thread and at least one inverted thread in order to increase bone fixation and load sharing between a bone/fastener interface during multi-axial and off-loading conditions to reduce high bone strain and distribute multi-axial forces applied to the bone in a load-sharing, rather than load-bearing, configuration. Shear loads and/or bending moments may also be optimally resisted with any chosen combination of threading, threading morphology, and/or threading variations contemplated herein to optimally resist shear loads, bending moments, multi-axial loading, off-loading conditions, etc.

In some embodiments, fasteners with standard threads may be used in conjunction with fasteners with inverted threads in order to accommodate different loading patterns.

In some embodiments, a single fastener may have both standard and inverted threads, like the fastener 100. Such a combination of threads may help the fastener 100 remain in place with unknown and/or varying loading patterns.

In some embodiments, the geometry of the threading of a fastener (with standard and/or inverted threads) may be varied to suit the fastener for a particular loading scheme. For example, the number of threads, the number of thread starts, the pitch of the threading, the lead(s) of the threading, the shape(s) of the threading, associated the any dimension(s) with threading (e.g., any length(s)/width(s)/height(s)/inflection-point(s), etc. associated with the threading), the major diameter(s), the minor diameter(s), any angulation/angles associated with any surfaces of the threading, the “handedness” of the threading (e.g., right-handed vs. left-handed), etc., may be varied accordingly to suit any specific medium of installation, loading pattern, desired radial loading force, pull-out strength, application, procedure, etc., that may be involved.

In some embodiments, the material(s) of any portion of a fastener, implant, or instrument described or contemplated herein may include, but are not limited to: metals (e.g., titanium, cobalt, stainless steel, etc.), metal alloys, plastics, polymers, ceramics, PEEK. UHMWPE, composites, additive particles, textured surfaces, biologics, biomaterials, bio-resorbable materials, bone, etc. Likewise, any fastener, implant, or instrument described or contemplated herein may be formed by any manufacturing method including, but not limited to: CNC manufacturing, injection molding, 3D printing, etc., and may include a solid or porous surface to encourage tissue/bone in-growth and/or may include any coating or surface treatment to stimulate tissue/bone growth, exhibit anti-microbial properties, etc.

In some embodiments, any of the fasteners or implants described herein may include additional features such as: self-tapping features, locking features (e.g., locking threading formed on a portion of the fastener, such as threading located on or near a head of the fastener), opening(s), longitudinal passageways, cannulation(s), fenestration(s), any style of fastener head (or no fastener head at all), any style of torque connection interface (or no torque connection interface at all), etc.

In some embodiments, any of the fasteners or implants described herein may also include opening(s), cannulation(s), fenestration(s), etc., that may be configured to receive any suitable bone cement or bone augment material therein to facilitate bone in-growth, bone fusion, etc.

In some embodiments, a tap tool may be utilized to pre-form threading in a bone or bone augment material according to any threading shape that is disclosed or contemplated herein. In this manner, tap tools with any suitable shape may be utilized in conjunction with any fastener described or contemplated herein to match or substantially match the threading geometry of a given fastener or implant.

In some embodiments, a minor diameter of the fastener may be selected to match, or substantially match, a diameter of a pilot hole that is formed in a bone to avoid bone blowout when the fastener is inserted into the pilot hole.

Additionally, or alternatively thereto, the type of threads and/or thread geometry may be varied based on the type of bone in which the fastener is to be anchored. For example, fasteners anchored in osteoporotic bone may fare better with standard or inverted threads, or when the pitch, major diameter, and/or minor diameter are increased or decreased, or when the angulation of thread surfaces is adjusted, etc.

In some embodiments, a surgical kit may include one or more fasteners (with any of the different thread options described or contemplated herein), bone implants, femoral fasteners, femoral support members, stop members, etc., as well as any of the instruments that are described or contemplated herein. The surgeon may select the appropriate components from the kit based on the particular loads to be applied and/or the quality of bone in which the implants(s) are to be anchored.

Continuing with FIG. 1D, in some embodiments the first helical thread 110 may include a plurality of first undercut surfaces 111, a plurality of second undercut surfaces 112, a plurality of third undercut surfaces 113, and a plurality of fourth open surfaces 114.

In some embodiments, the second helical thread 120 may include a plurality of fifth undercut surfaces 125, a plurality of sixth undercut surfaces 126, a plurality of seventh undercut surfaces 127, and a plurality of eighth open surfaces 128.

In some embodiments one or more of the plurality of first undercut surfaces 111, the plurality of second undercut surfaces 112, the plurality of third undercut surfaces 113, the plurality of fourth open surfaces 114, the plurality of fifth undercut surfaces 125, the plurality of sixth undercut surfaces 126, the plurality of seventh undercut surfaces 127, and the plurality of eighth open surfaces 128 may comprise at least one flat or substantially flat surface.

In some embodiments, the plurality of first undercut surfaces 111, the plurality of third undercut surfaces 113, the plurality of sixth undercut surfaces 126, and the plurality of eighth open surfaces 128 may be angled towards the distal end 102 of the shaft 105.

In some embodiments, the plurality of second undercut surfaces 112, the plurality of fourth open surfaces 114, the plurality of fifth undercut surfaces 125, and the plurality of seventh undercut surfaces 127 may be angled towards the proximal end 101 of the shaft 105.

In some embodiments, when the fastener 100 is viewed in section along a plane that intersects the longitudinal axis 103 of the shaft 105 (as shown in FIG. 1D), the first helical thread 110 may include at least one chevron shape that is oriented toward (i.e., points toward) the distal end 102 of the shaft 105. Likewise, the second helical thread 120 may also include at least one chevron shape that is oriented toward (i.e., points toward) the proximal end 101 of the shaft 105.

In some embodiments, when the fastener 100 is viewed in section along a plane that intersects the longitudinal axis 103 of the shaft 105 (as shown in FIG. 1D), the first helical thread 110 may include a first plurality of chevron shapes that are oriented toward (i.e., point toward) the distal end 102 of the shaft 105. Likewise, the second helical thread 120 may include a second plurality of chevron shapes that are oriented toward (i.e., point toward) the proximal end 101 of the shaft 105.

In some embodiments, the first plurality of chevron shapes and the second plurality of chevron shapes may be arranged in alternating succession along the shaft 105 of the fastener 100, (e.g., see FIG. 1D).

In some embodiments, a plurality of first interlocking spaces 161 and a plurality of second interlocking spaces 162 may be formed between the first helical thread 110 and the second helical thread 120 along the shaft 105 of the fastener 100.

In some embodiments, the plurality of first interlocking spaces 161 may be formed intermediate the first concave undercut surfaces 131 and the second concave undercut surfaces 132.

In some embodiments, the plurality of second interlocking spaces 162 may be formed intermediate the first convex undercut surfaces 141 and the second convex undercut surfaces 142.

In some embodiments, the plurality of first interlocking spaces 161 may be larger in size than the plurality of second interlocking spaces.

In some embodiments, the plurality of first interlocking spaces 161 and the plurality of second interlocking spaces 162 may be shaped and/or configured to interlock with bone/other tissues received therein to increase fixation of the fastener 100 within the bone/other tissues and provide additional resistance against multi-axial forces that may be applied to the fastener 100 and/or the bone/other tissues.

In some embodiments, the plurality of second undercut surfaces 112 and the plurality of sixth undercut surfaces 126 may be angled toward each other to trap bone, soft tissues, other tissues, bone augment material(s), etc., within the plurality of first interlocking spaces 161 in order to increase fixation and resistance against multi-axial forces.

In some embodiments, the plurality of third undercut surfaces 113 and the plurality of seventh undercut surfaces 127 may be angled toward each other to trap bone, soft tissues, other tissues, bone augment material(s), etc., within the plurality of second interlocking spaces 162 in order to increase fixation and resistance against multi-axial forces.

In some embodiments, the plurality of first undercut surfaces 111 and the plurality of fifth undercut surfaces 125 may each form an angle α with respect to the longitudinal axis 103 of the shaft 105, as shown in FIG. 1D.

In some embodiments, the angle α may be greater than 90 degrees.

In some embodiments, the plurality of second undercut surfaces 112 and the plurality of sixth undercut surfaces 126 may each form an angle β with respect to the longitudinal axis 103 of the shaft 105.

In some embodiments, the angle β may be less than 90 degrees.

In some embodiments, the plurality of third undercut surfaces 113 and the plurality of seventh undercut surfaces 127 may each form an angle θ with respect to the longitudinal axis 103 of the shaft 105.

In some embodiments, the angle θ may be approximately 90 degrees.

In some embodiments, the angle θ may be greater than 90 degrees.

It will be understood that any fastener, implant, etc., described or contemplated herein may include any thread configuration, feature, or morphology that is described or contemplated herein to achieve optimal fixation within a given bone/tissue. Moreover, it will also be understood that any fastener, implant, etc., described or contemplated herein may be utilized in conjunction with (or within) any system, method, or instrumentation that is described or contemplated herein.

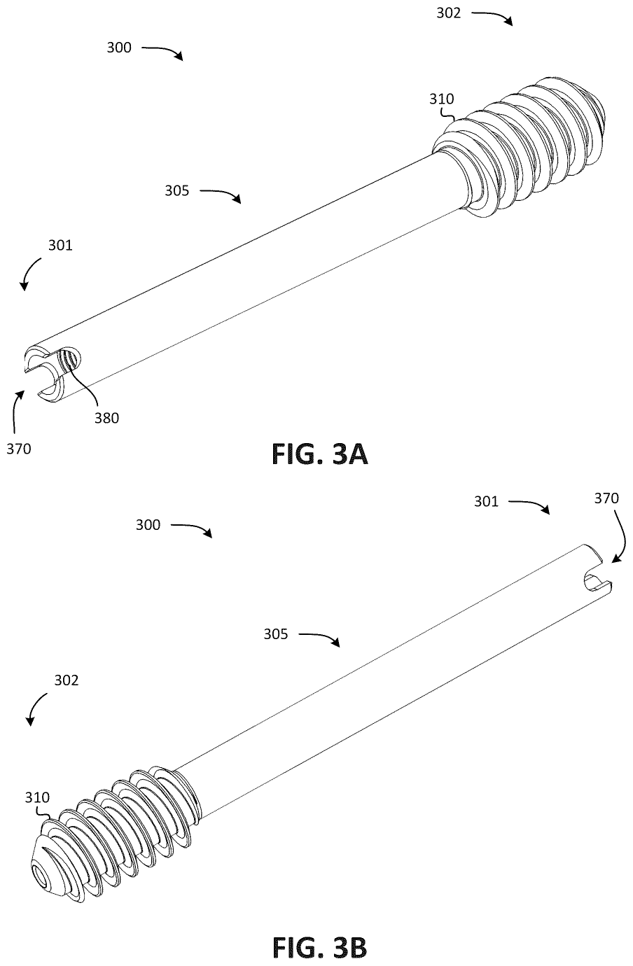

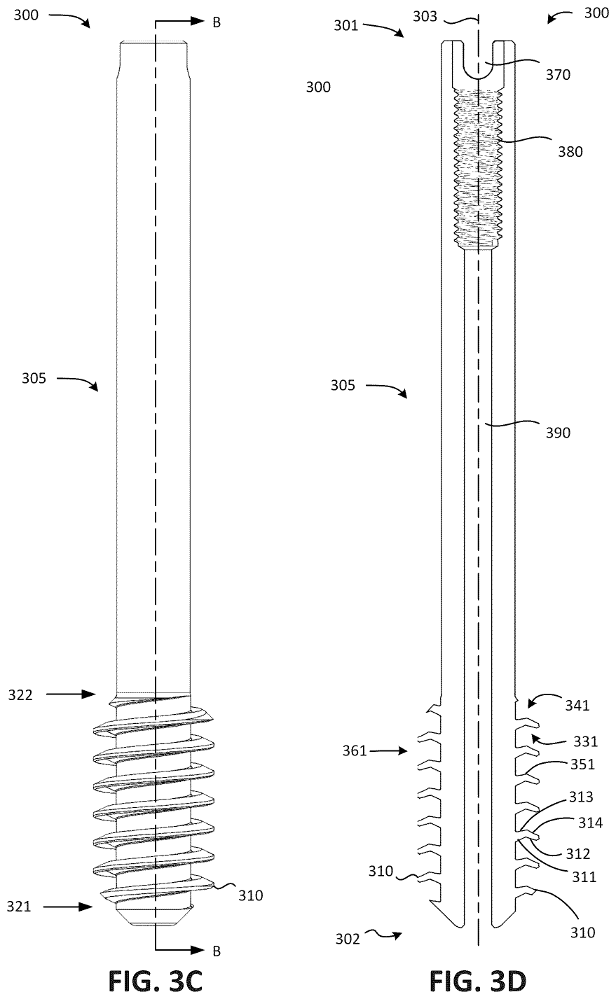

FIGS. 3A-D illustrate various views of a femoral fixation device or femoral fastener 300, according to another example of the present disclosure. Specifically, FIG. 3A is a rear perspective view of the femoral fastener 300, FIG. 3B is a front perspective view of the femoral fastener 300, FIG. 3C is a side view of the femoral fastener 300, and FIG. 3D is a cross-sectional side view of the femoral fastener 300 taken along the line B-B in FIG. 3C.

In general, the femoral fastener 300 may include a shaft 305 having a proximal end 301, a distal end 302, and a longitudinal axis 303, as well as a helical thread 310 disposed about at least a portion of the shaft 305.

In some embodiments, the shaft 305 of the femoral fastener 300 may be cannulated with a through bore or longitudinal passageway 390.

In some embodiments, the longitudinal passageway 390 may include an internal thread 380 formed along at least a portion of a length of the longitudinal passageway 390.

In some embodiments, the internal thread 380 may be located toward the proximal end 301 of the shaft 305.

In some embodiments, the proximal end 301 of the femoral fastener 300 may comprise a headless fastener design having an at least partially cylindrical shape.

In some embodiments, the proximal end 301 of the femoral fastener 300 may include one or more recesses 370 which may extend along the shaft from the proximal end 301 of the shaft 305 toward the distal end 302 of the shaft 305.

In some embodiments, the one or more recesses 370 may be shaped and configured to couple with an inserter tool to form a torque connection interface that may facilitate insertion of the femoral fastener 300, as will be discussed in more detail below.

In some embodiments, the femoral fastener 300 may include one or more self-tapping or bone cutting features formed in a distal portion of the femoral fastener 300 (not shown).

In some embodiments, the helical thread 310 may be disposed about the shaft 305 along the longitudinal axis 303 between a first location 321 and a second location 322 along the shaft 305.

Although the femoral fastener 300 shown in FIGS. 3A-3D illustrates a single helical thread design, it will be understood that the femoral fastener 300 may include any number of threads and/or any number of thread characteristics, shapes, or configurations that are described or contemplated herein, in any combination. For example, the femoral fastener 300 may include a “dual start” or “dual lead” thread configuration comprising a first helical thread, and a second helical thread as previously described herein, etc.

In some embodiments, a depth of the helical thread 310 with respect to the shaft 305 may define a major diameter vs. a minor diameter of the shaft 305 alone.

In some embodiments, the major diameter, the minor diameter, and/or a pitch of the helical thread 310 may be constant or substantially constant along a length of the femoral fastener 300.

In some embodiments, the helical thread 310 may include one or more concave undercut surfaces 331 and/or one or more convex undercut surfaces 341.

In some embodiments, the one or more concave undercut surfaces 331 may be angled towards one of the proximal end 301 and the distal end 302 of the shaft 305.

In some embodiments, the one or more convex undercut surfaces 341 may be angled towards the other one of the proximal end 301 and the distal end 302 of the shaft 305.

In some embodiments, the one or more concave undercut surfaces 331 may be angled towards the proximal end 301 of the shaft 305 and the one or more convex undercut surfaces 341 may be angled towards the distal end 302 of the shaft 305.

In some embodiments, the one or more concave undercut surfaces 331 and/or the one or more convex undercut surfaces 341 may include a plurality of flat surfaces that are angled relative to each other.

In some embodiments, the helical thread 310 may include one or more first undercut surfaces 311 and one or more second undercut surfaces 312.

In some embodiments, the one or more first undercut surfaces 311 may be angled toward the proximal end 301 of the shaft 305 and one or more second undercut surfaces 312 may be angled toward the distal end 302 of the shaft 305.

In some embodiments, the helical thread 310 may also include one or more third undercut surfaces 313 and one or more fourth open surfaces 314. However, it will be understood that in other embodiments the one or more third undercut surfaces 313 and the one or more fourth open surfaces 314 may be replaced with any other shaped surface or surfaces (e.g., any buttress type thread shape, any flat surface that is angled toward or away from the one or more concave undercut surfaces 331, or angled 90 degrees with respect thereto, any curved surface that is generally oriented toward or away from the one or more concave undercut surfaces, etc.) without departing from the spirit or scope of the present disclosure.

In some embodiments, when the femoral fastener 300 is viewed in section along a plane intersecting the longitudinal axis 303 of the shaft 305, the helical thread 310 may include at least one chevron shape oriented toward the proximal end 301 of the shaft 305.

In some embodiments, when the femoral fastener 300 is viewed in section along a plane intersecting the longitudinal axis 303 of the shaft 305, the helical thread 310 may include a plurality of chevron shapes oriented toward the proximal end 301 of the shaft 305.

In some embodiments, when the femoral fastener 300 is viewed in section along a plane intersecting the longitudinal axis 303 of the shaft 305, the helical thread 310 may include at least one partial crescent shape oriented toward the proximal end 301 or the distal end 302 of the shaft 305.

In some embodiments, when the femoral fastener 300 is viewed in section along a plane intersecting the longitudinal axis 303 of the shaft 305, the helical thread 310 may include a plurality of partial crescent shapes oriented toward the proximal end 301 or the distal end 302 of the shaft 305.

In some embodiments, when the femoral fastener 300 is implanted within a neck 710 and a head 720 of a femoral bone or femur 700, the first location 321, the second location 322, and the helical thread 310 extending therebetween may be disposed within the head 720 of the femur 700.

In some embodiments, when the femoral fastener 300 is implanted within a neck 710 and a head 720 of a femur 700, at least one of: the one or more concave undercut surfaces 331, the one or more convex undercut surfaces 341, the one or more first undercut surfaces 311, the one or more second undercut surfaces 312, the one or more third undercut surfaces 313, and/or the one or more fourth open surfaces 314 may be configured to transmit at least one force from the head 720 of the femur 700 to the neck 710 (or other portion) of the femur 700. In this manner, the unique shape and configuration of the helical thread 310 can help mitigate or prevent loosening of the femoral fastener 300 over time due to multi-axial forces and off-axis loading scenarios that may be applied to the femoral fastener 300.

In some embodiments, one or more interlocking spaces 361 may be formed between adjacent thread portions of the helical thread 310 along the shaft 305 of the femoral fastener 300.

In some embodiments, the one or more interlocking spaces 361 may be shaped and/or configured to interlock with bone/other tissues received therein to increase fixation of the femoral fastener 300 within the bone/other tissues and provide additional resistance against multi-axial forces that may be applied to the femoral fastener 300 and/or the bone/other tissues.

In some embodiments, when the femoral fastener 300 is viewed in section along a plane intersecting the longitudinal axis 303 of the shaft 305, the helical thread 310 may include one or more bent shapes (comprising at least one surface that is angled relative to the longitudinal axis 303 of the shaft 305 and/or at least one undercut surface) with one or more intermediate portions 351 that are oriented toward (i.e., point toward) one of the proximal end 301 and the distal end 302 of the shaft 305.

In some embodiments, at least one of: the one or more concave undercut surfaces 331, the one or more convex undercut surfaces 341, the one or more first undercut surfaces 311, the one or more second undercut surfaces 312, the one or more third undercut surfaces 313, and/or the one or more fourth open surfaces 314 may comprise at least one substantially flat surface.

In some embodiments, at least one of: the one or more concave undercut surfaces 331, the one or more convex undercut surfaces 341, the one or more first undercut surfaces 311, the one or more second undercut surfaces 312, the one or more third undercut surfaces 313, and/or the one or more fourth open surfaces 314 may comprise at least one curved surface.

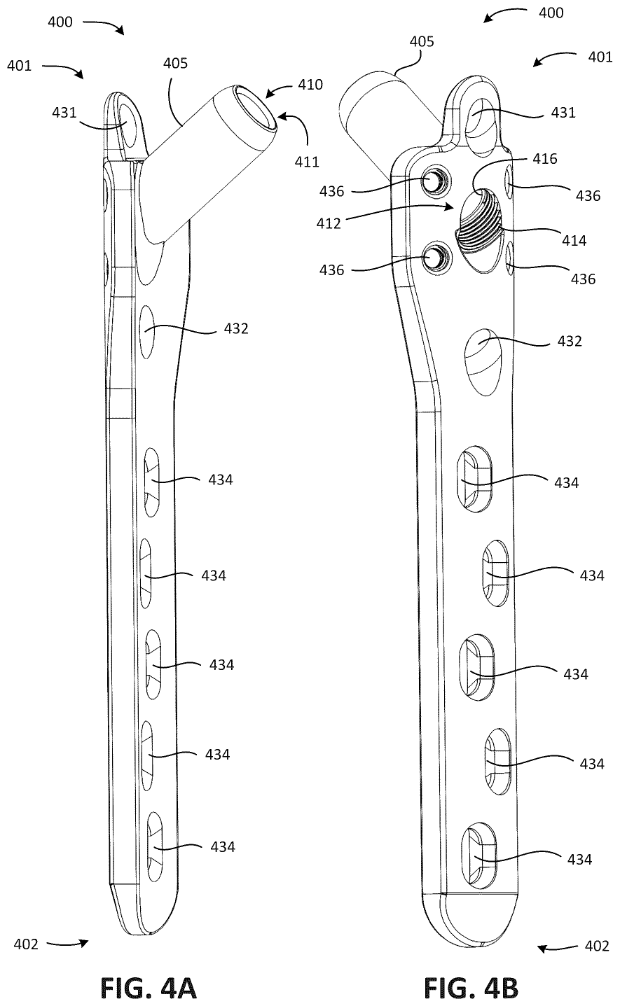

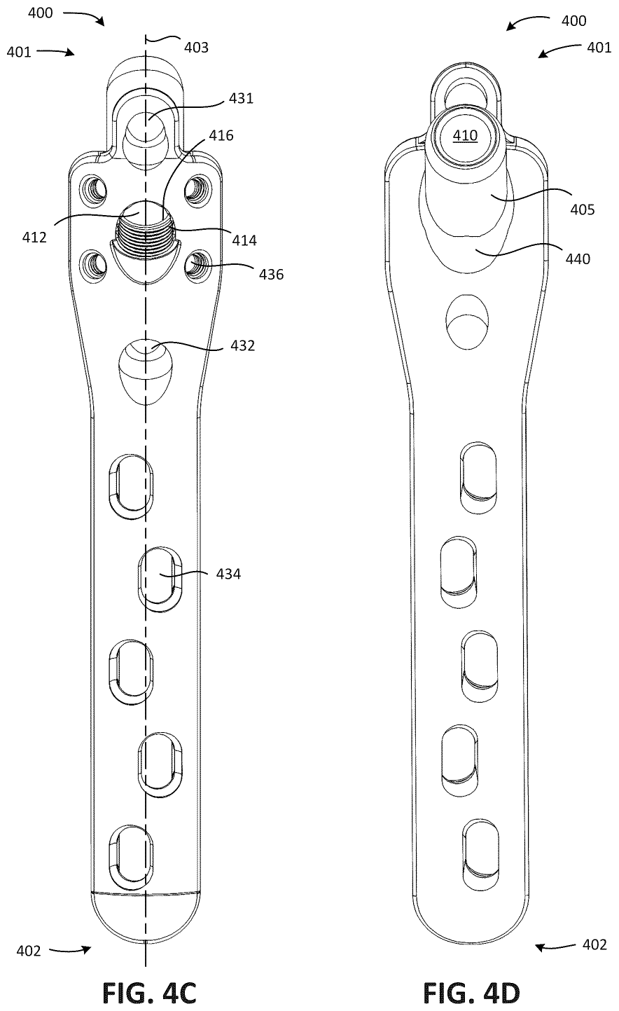

FIGS. 4A-D illustrate various views of a femoral support member 400, according to an example of the present disclosure. Specifically, FIG. 4A is a perspective side view of the femoral support member 400, FIG. 4B is another perspective side view of the femoral support member 400, FIG. 4C is a front view of the femoral support member 400, and FIG. 4D is a rear view of the femoral support member 400.

In general, the femoral support member 400 may include an elongate body having a proximal end 401, a distal end 402, a longitudinal axis 403, and a barrel 405.

In some embodiments, the femoral support member 400 may comprise a bone plate.

In some embodiments, the femoral support member 400 may comprise an intramedullary nail (not shown).

In some embodiments, the femoral support member 400 may include one or more bone plate apertures 434, one or more inferior support apertures 432, one or more superior support apertures 431, and/or one or more extension plate apertures 436.

In some embodiments, the barrel 405 may include a passageway 410 formed therethrough having a first opening 411 and a second opening 412 opposite the first opening 411.

In some embodiments, the passageway 410 may also include an internal thread 414 and a barrel shoulder 416 located adjacent the internal thread 414.

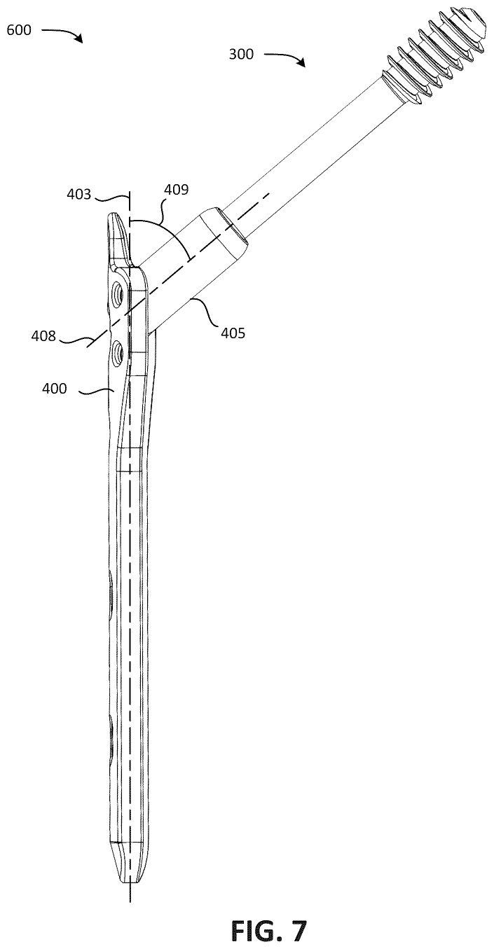

In some embodiments, the barrel 405 and/or the passageway 410 may be formed through the femoral support member 400 with a longitudinal axis 408 at an angle 409 with respect to the longitudinal axis 403 of the femoral support member 400, as shown in FIG. 7.

In some embodiments, the angle 409 of the barrel 405 and/or the passageway 410 with respect to the longitudinal axis 403 of the femoral support member 400 may be an acute angle. However, it will also be understood that in some embodiments the angle 409 may be a right angle and/or an obtuse angle.

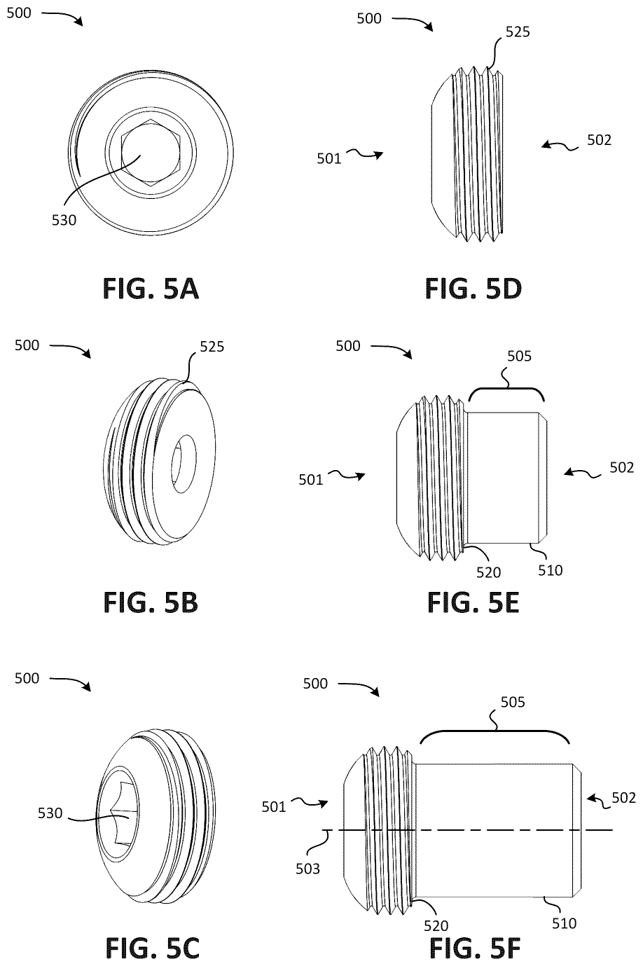

FIGS. 5A-F illustrate various views of a stop member 500, according to an example of the present disclosure. Specifically. FIG. 5A is a front view of the stop member 500, FIG. 5B is a rear perspective view of the stop member 500, FIG. 5C is a front perspective view of the stop member 500, FIG. 5D is a side view of the stop member 500, and FIGS. 5E and 5F are side views of the stop member 500 including a stop member projection 510 having a preselected length 505.

In general, the stop member 500 may include a proximal end 501, a distal end 502, and a longitudinal axis 503.

In some embodiments, the stop member 500 may include a torque connection interface 530. In some embodiments, the torque connection interface 530 may comprise a hexagonal shape. However, it will be understood that the torque connection interface 530 may comprise any shape suitable for receiving a torque force from a driver tool, as will be discussed in more detail below.

In some embodiments, the stop member 500 may include an external thread 525.

In some embodiments, the stop member 500 may include a stop member shoulder intermediate the external thread 525 and the stop member projection 510.

In some embodiments, the preselected length 505 of the stop member projection 510 may be zero.

In some embodiments, the preselected length 505 of the stop member projection 510 may be greater than zero.

In some embodiments, the preselected length 505 of the stop member projection 510 may be 3 mm, 5 mm, 10 mm, etc., as some non-limiting examples of a preselected length 505 that is greater than zero. However, it will be understood that any length greater than or equal to zero may be utilized for the preselected length 505 of the stop member projection 510.

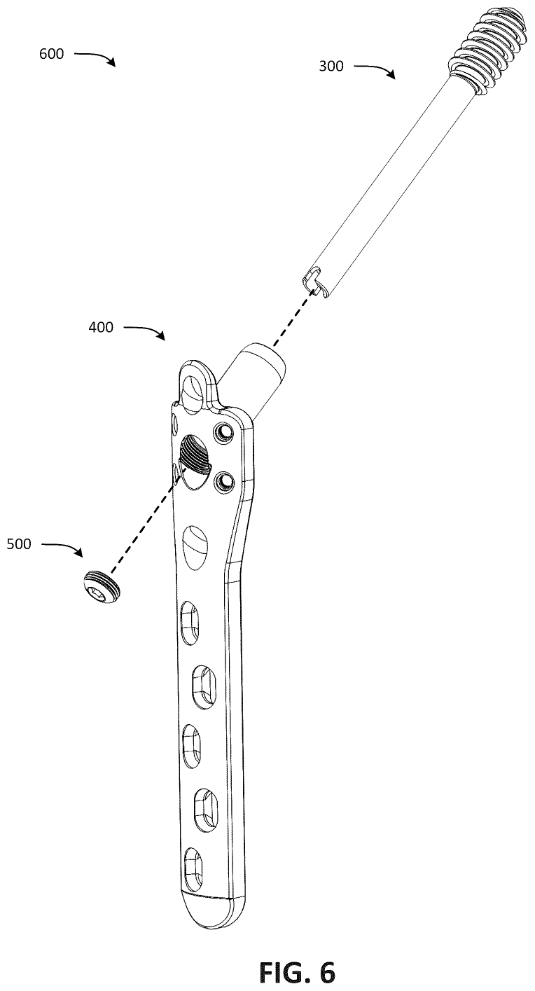

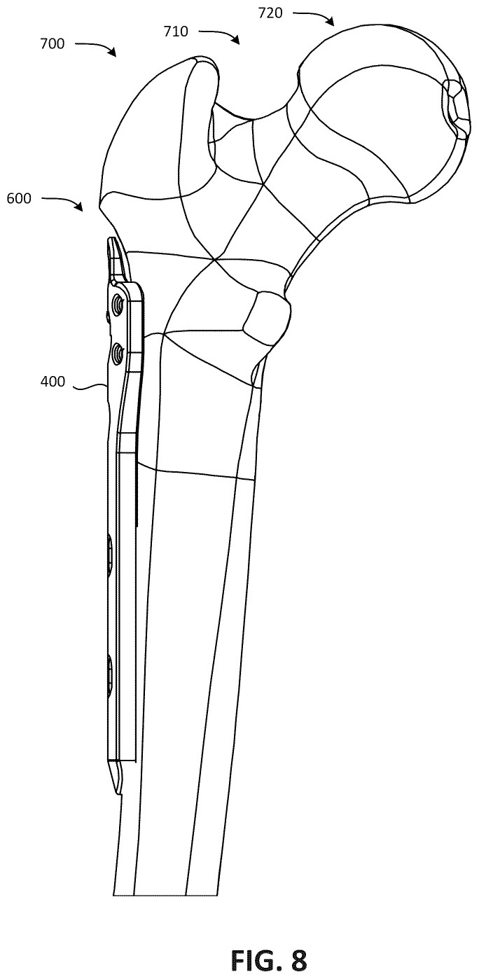

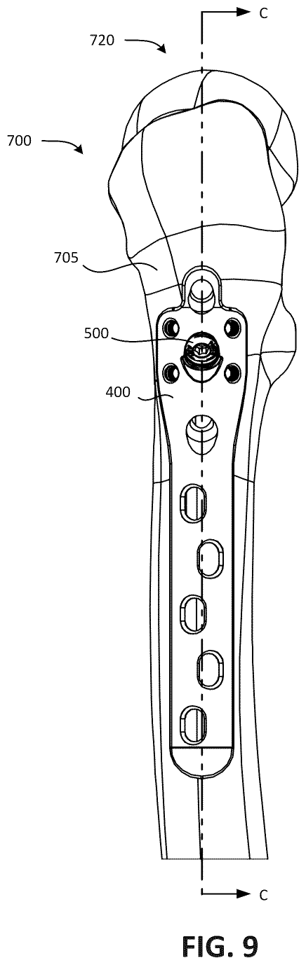

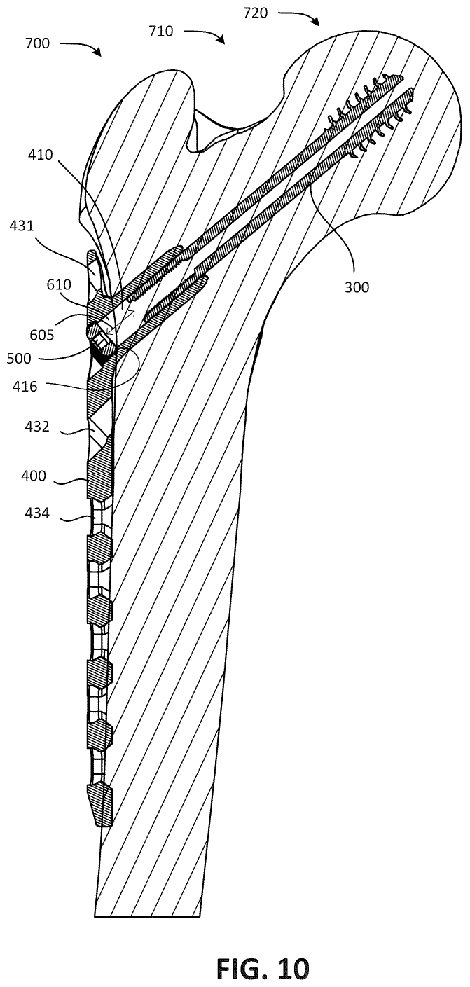

FIGS. 6-10 illustrate various views of a femoral fixation assembly 600 comprising the femoral support member 400, the femoral fastener 300, and the stop member 500, according to an embodiment of the present disclosure. Specifically, FIG. 6 illustrates an exploded view of the femoral fixation assembly 600; FIG. 7 illustrates a side view of the femoral fixation assembly 600 after assembly; FIG. 8 illustrates a side view of the femoral fixation assembly 600 implanted into a femur 700; FIG. 9 illustrates a front view of the femoral fixation assembly 600 and femur 700 of FIG. 8; and FIG. 10 illustrates cross-sectional side view of the femoral fixation assembly 600 and femur 700 of FIG. 9 taken along the line C-C.

With reference to FIG. 10, when the femoral fastener 300 is implanted within the neck 710 and the head 720 of the femur 700, and the femoral support member 400 is oriented with respect to a longitudinal axis of the femoral bone, at least a portion of the shaft 305 of the femoral fastener 300 may be slidingly received within the passageway 410 of the barrel 405 through the first opening 411 of the passageway 410.

In this manner, the femoral fastener 300 may be allowed to slide further into the passageway 410 of the barrel 405 as the femur 700 collapses due to the bone remodeling process, the healing process, continued use of the femur over time, etc.

However, it may be desirable to limit the amount of collapse that the femur 700 may undergo. Accordingly, in some embodiments, the stop member 500 may be inserted into the passageway 410 in order to set a predetermined limit to the amount of collapse that the femur 700 may be permitted to undergo.

In some embodiments, at least a portion of the stop member 500 may be received within the passageway 410 through the second opening 412.

In some embodiments, the stop member 500 may be inserted into the passageway 410 and coupled thereto by engaging the external thread 525 of the stop member 500 with the internal thread 380 of the femoral fastener 300.

In some embodiments, a stop member shoulder 520 of the stop member 500 may be configured to abut against a barrel shoulder 416 of the passageway 410 when the stop member 500 has been fully inserted into the passageway 410.

In some embodiments, a space 610 having a predetermined length 605 may be formed within the passageway 410 between the distal end 502 of the stop member 500 and the proximal end 301 of the shaft 305 based on the preselected length 505 of the stop member projection 510. The predetermined length 605 of the space 610 may define the amount of collapse that the femur 700 may be permitted to undergo.

In some embodiments, the preselected length 505 of the stop member projection 510 may be selected such that the predetermined length 605 of the space 610 within the passageway 410 may be zero. In these embodiments, the distal end 502 of the stop member 500 may abut against the proximal end 301 of the femoral fastener 300 to prevent collapse.

In some embodiments, the preselected length 505 of the stop member projection 510 may be selected such that the predetermined length 605 of the space 610 within the passageway 410 may be greater than zero. In these embodiments, the predetermined length 605 of the space 610 in the passageway 410 may define the amount of collapse that the femur 700 may be permitted to undergo.

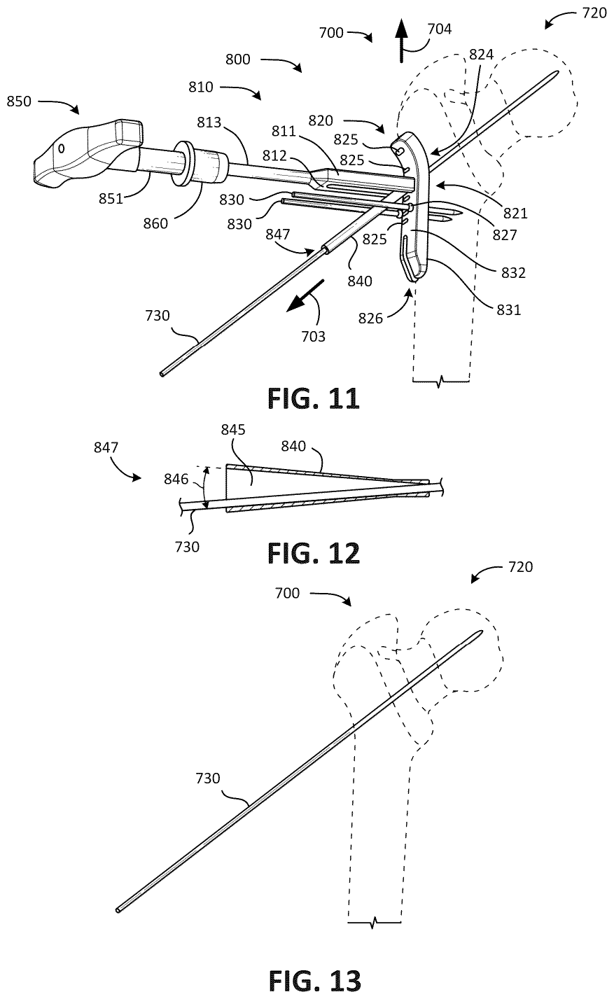

FIGS. 11-35 illustrate various views of a surgical procedure that may be utilized to install the femoral fixation assembly 600 into a femur 700.

FIG. 11 illustrates a perspective side view of a guide pin inserter assembly or targeting guide assembly 800 placed adjacent a femur 700, according to an embodiment of the present disclosure.

The targeting guide assembly 800 may generally include a handle 850, a side plate positioner or targeting guide positioner 810 coupled to the handle 850, a side plate guide or targeting guide base 820 coupled to the targeting guide positioner 810, and a guide pin insert or targeting guide insert 840 coupled to the targeting guide base 820.

In some embodiments, one or more of the handle 850, the targeting guide positioner 810, the targeting guide base 820, and/or the targeting guide insert 840 may each be removably couplable with each other.

In some embodiments, one or more of the handle 850, the targeting guide positioner 810, the targeting guide base 820, and/or the targeting guide insert 840 may be integrally formed with each other. For example, in some embodiments the targeting guide positioner 810 and the targeting guide base 820 may be integrally formed with each other.

The targeting guide base 820 may be placed against the lateral side of the femur 700 by manipulating the handle 850 to orient the targeting guide base 820 with respect to a longitudinal axis of the femur. Once the targeting guide base 820 has been properly located adjacent the lateral side of the femur 700, one or more placement pins 830 (e.g., olive pins, etc.) may be inserted through apertures or one or more placement pin holes 827 formed in the targeting guide base 820 to pin the targeting guide base 820 to the femur 700.

Once the targeting guide base 820 has been pinned to the femur 700 at a desired location, the targeting guide insert 840 may be coupled to the targeting guide base 820 by inserting a distal end of the targeting guide insert 840 into the or one or more locating slots or locating slot 825 of the targeting guide base 820, depending on a desired superior/inferior location and/or trajectory for a K-wire or guide pin 730.

In some embodiments, an interior space 845 of the targeting guide insert 840 may be substantially straight and/or cylindrical in shape in order to direct the guide pin 730 along a single trajectory into the head 720 of the femur 700.

In some embodiments, the interior space 845 of the targeting guide insert 840 may be flared, oval, and/or somewhat conical in shape in order to allow the surgeon some degree of latitude to choose a trajectory for the guide pin 730 into the head 720 of the femur 700.

In some embodiments, the interior space 845 of the targeting guide insert 840 may include an adjustable angle 846 that may allow the trajectory of the guide pin 730 to be varied in at least an anterior-posterior direction, but it will be understood that, in at least some embodiments, the trajectory of the guide pin 730 can be varied in any other direction(s), and/or in all directions.

In some embodiments, the adjustable angle 846 may span a range of about zero to nine degrees, as one non-limiting example. However, it will be understood that the interior space 845 may comprise any angle or range of angles (e.g., a range zero to fifteen degrees, etc.).

In some embodiments, once the guide pin 730 has been properly placed within the head 720 of the femur 700, the targeting guide assembly 800 may be removed from the femur without disturbing the guide pin 730 by sliding the targeting guide base 820 superiorly. In these embodiments, the targeting guide insert 840 may be removed from the guide pin 730. For example, the targeting guide insert 840 may be configured to detach from the targeting guide base 820 and slidingly disengage from the guide pin 730 in a proximal direction 703.

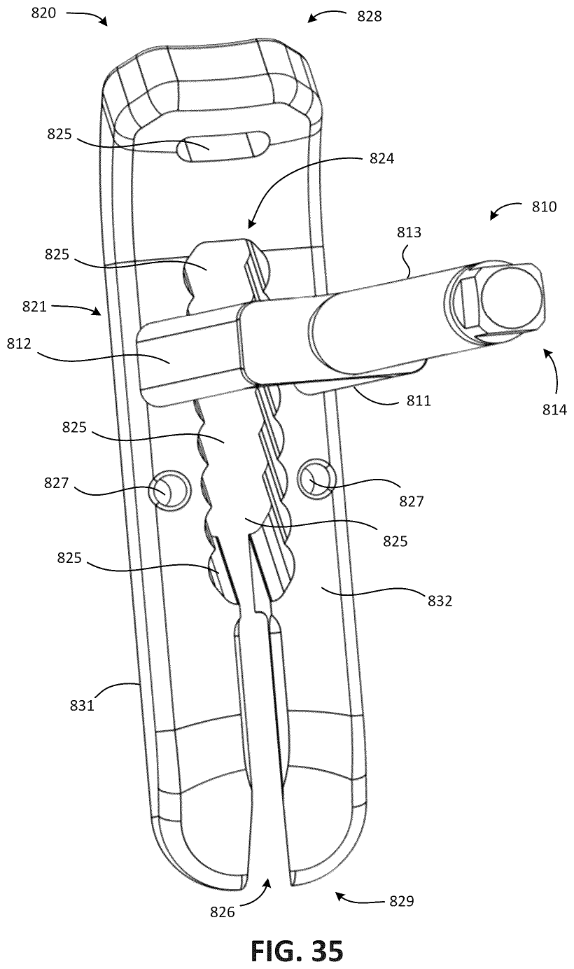

The targeting guide base 820 may then be moved superiorly, or along a superior direction 704, to allow the guide pin 730 to exit the targeting guide base 820 inferiorly through the removal slot 826 that is formed in the targeting guide base 820 and in communication with the locating slot 825 that is also formed in the targeting guide base 820. In these embodiments, the removal slot 826 may extend further superiorly (than what is illustrated in FIG. 11) in order to provide a channel through which the guide pin 730 may exit the targeting guide base 820 inferiorly as the targeting guide base 820 is moved superiorly. For example, FIG. 35 illustrates a targeting guide base 820 comprising a removal slot 826 extending superiorly to connect with the locating slot 825. In this manner, the guide pin 730 may be allowed to exit the targeting guide base 820 inferiorly through the removal slot 826 as the targeting guide base 820 is moved superiorly.

FIG. 13 illustrates a side view of a guide pin 730 located in the femur 700 after the targeting guide assembly 800 has been removed.

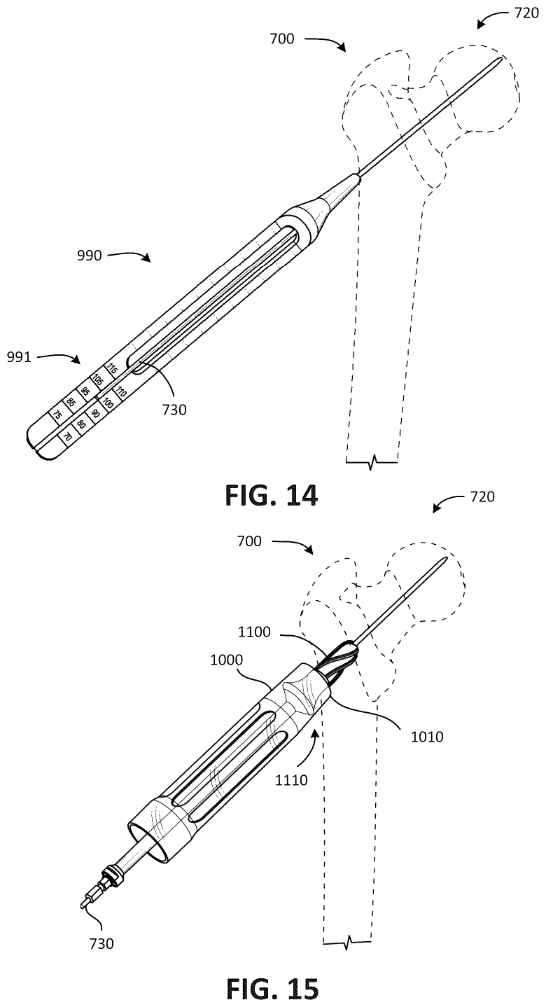

FIG. 14 illustrates a side view of a guide pin depth gauge 990 adjacent the femur 700 in order to measure a depth of the guide pin 730 inside the femur 700. The guide pin depth gauge 990 may include one or more markings 991 configured to indicate a depth of the guide pin 730 inside the femur 700, which may then be utilized to determine a selected length for the femoral fastener 300.

Once the depth of the guide pin 730 has been determined, a reamer or reamer tool 1100 and a tissue shield 1000 may be placed adjacent the femur 700 (e.g., see FIG. 15) in order to ream a bone tunnel in the femur 700 that may be shaped to receive the barrel 405 of the femoral support member 400 therein.

In some embodiments, a distal end 1010 of the tissue shield 1000 may be notched to conform to a lateral surface 705 of the femur 700.

In some embodiments, the reamer tool 1100 may include a flared portion 1110 that may be configured to provide a countersink in the femur to receive the base 440 of the barrel 405 therein (see FIG. 4D).

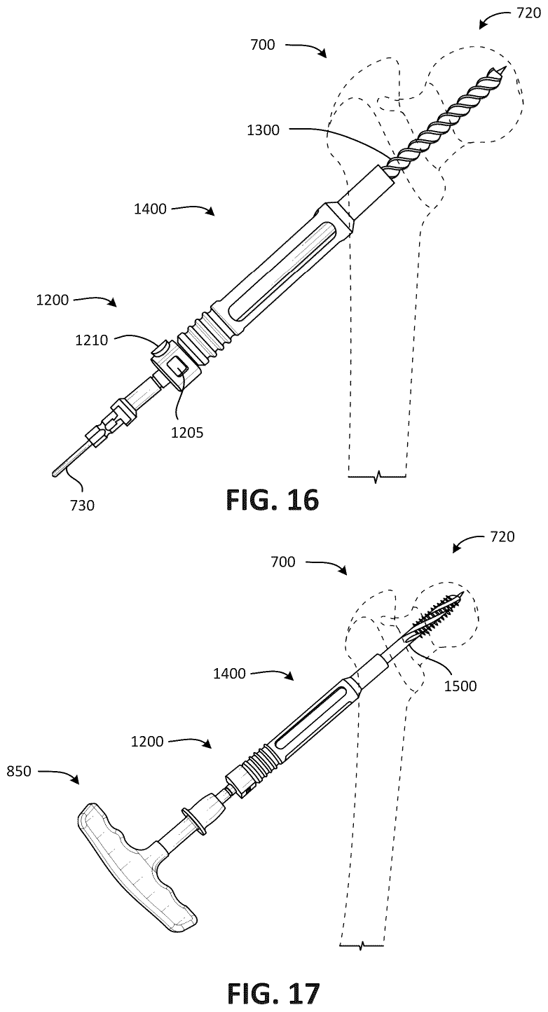

FIG. 16 illustrates a side view of drill bit 1300, an adjustable depth stop 1200, and centering sleeve 1400, according to embodiments of the present disclosure. The adjustable depth stop 1200 may be slid onto the drill bit 1300 while depressing the button 1210 on the adjustable depth stop 1200 until a previously measured femoral fastener length is seen in the window 1205. The button 1210 may then be released in order to couple the adjustable depth stop 1200 to the drill bit 1300 and hold its position. The centering sleeve 1400 may be slid over the drill bit 1300. The drill bit 1300 and centering sleeve 1400 may then be placed over the guide pin 730. The centering sleeve 1400 may be slid down the guide pin 730 until seated, and the drill bit 1300 may then be rotated to form a bone tunnel for the femoral fastener 300. The drill bit 1300 may be advanced until the adjustable depth stop 1200 rests against the centering sleeve 1400. This may indicate that the bone tunnel has been formed to a proper depth corresponding to the depth shown in the window 1205 of the adjustable depth stop 1200.

FIG. 17 illustrates a side view of a tap tool or tap 1500, according to an embodiment of the present disclosure. The adjustable depth stop 1200 may be placed on the tap 1500 in the same manner as the drill bit 1300 described above. The tap 1500 and centering sleeve 1400 may then be placed over the guide pin 730 and advanced into the femur. The tap 1500 may then be rotated using the handle 850 to form a tapped bone thread about the bone tunnel in the head 720 of the femur 700. The tapped bone thread may be configured to receive the helical thread 310 of the femoral fastener 300 therein.

In some embodiments, the tap 1500 may be configured to pre-form threading in the femur 700 according to any threading shape that is disclosed herein. In this manner, taps with any suitable shape may be utilized in conjunction with any fastener described or contemplated herein to match or substantially match the threading geometry of a given fastener.

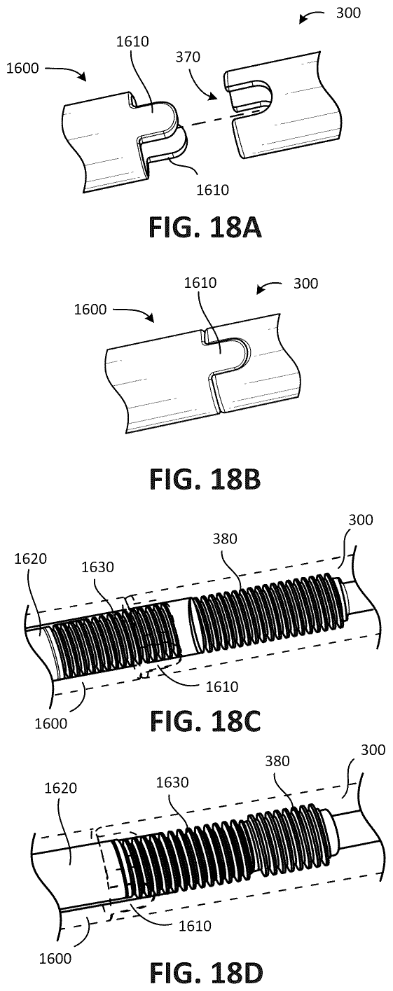

FIGS. 18A-18D illustrate a connection feature formed between an inserter tool 1600 and the femoral fastener 300, according to an embodiment of the present disclosure. Specifically, FIG. 18A is a close up partial view of the connection feature between the inserter tool 1600 and the femoral fastener 300 prior to engagement; FIG. 18B is a side view of the connection feature of FIG. 18A after engagement; FIG. 18C is a side view of the connection feature showing an inserter coupler 1620 inside the inserter tool 1600 before it engages with the internal thread 380 of the femoral fastener 300; and FIG. 18D illustrates a side view of the connection feature after the external thread 1630 of the inserter coupler 1620 engages the internal thread 380 of the femoral fastener 300 to retain the femoral fastener 300 to the inserter tool 1600. As shown in FIGS. 18A-18B, the inserter tool 1600 may include one or more projections 1610 that may be received within the one or more recesses 370 of the femoral fastener 300. In this manner, the femoral fastener 300 may be coupled with the inserter tool 1600 to facilitate insertion of the femoral fastener 300 into the bone tunnel formed in the femur 700.

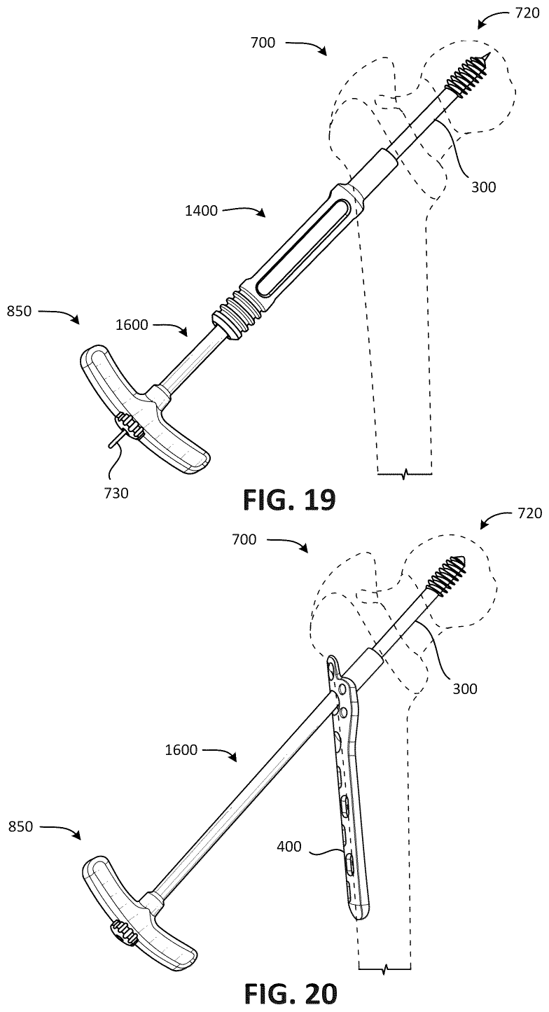

FIG. 19 illustrates a side view of the centering sleeve 1400 and the inserter tool 1600 facilitating placement of the femoral fastener 300 within the femur 700.

In some embodiments, a portion of the shaft 305 of the femoral fastener 300 comprising the helical thread 310 may be placed within the head 720 of the femur 700, such that, a concave undercut surface of the helical thread 310 may be positioned within the head 720 of the femur 700 to transmit at least one force from the head 720 of the femur 700 to the neck 710 of the femur 700.

In some embodiments, placing the a portion of the shaft 305 comprising the helical thread 310 within the head 720 of the femur 700 comprises rotating the shaft 305 to insert the helical thread 310 into a tapped bone thread that is disposed about the bone tunnel.

FIG. 20 illustrates a side view of the inserter tool 1600 facilitating placement of the femoral support member 400 against the femur 700 with the proximal end 301 of the shaft 305 of the femoral fastener 300 placed inside the passageway 410 of the barrel 405.

Thus, in some embodiments, the proximal end 301 of the shaft 305 of the femoral fastener 300 may be inserted into the first opening 411 of the passageway 410 formed through the femoral support member 400 when the femoral support member 400 is oriented with respect to the longitudinal axis of the femoral bone.

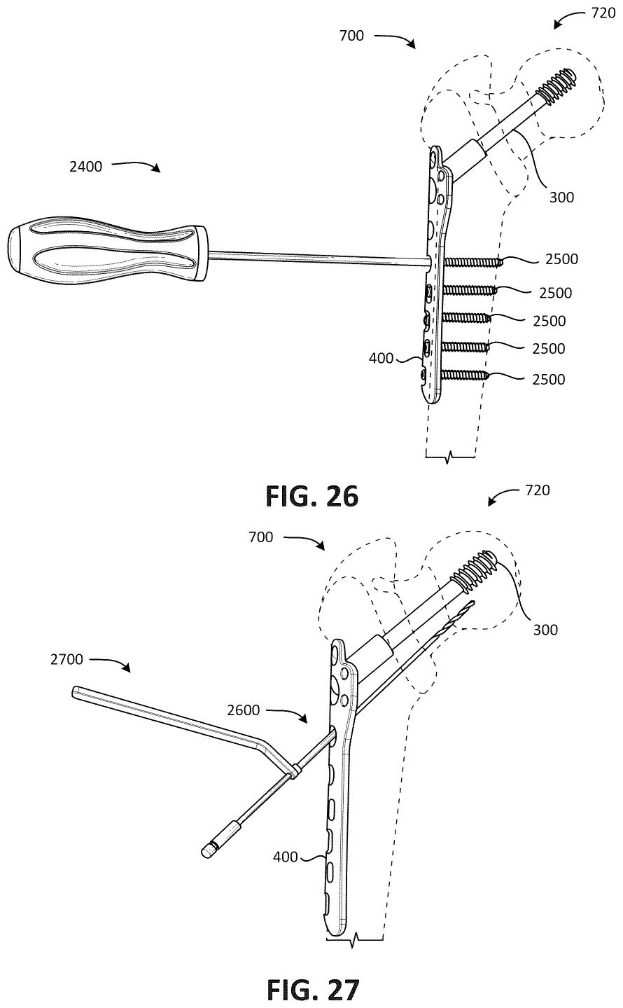

Moreover, in some embodiments the distal end 502 of the stop member 500 may be inserted into the second opening 412 of the passageway 410 opposite the first opening 411, such that a space 610 having a predetermined length 605 may be formed within the passageway 410 between the distal end 502 of the stop member 500 and the proximal end 301 of the shaft 305 based on the preselected length 505 of the stop member 500 or stop member projection 510. In some embodiments, the stop member 500 may be placed within the passageway 410 after the femoral support member 400 has been secured to the femur 700 (e.g., see FIGS. 26-34B).

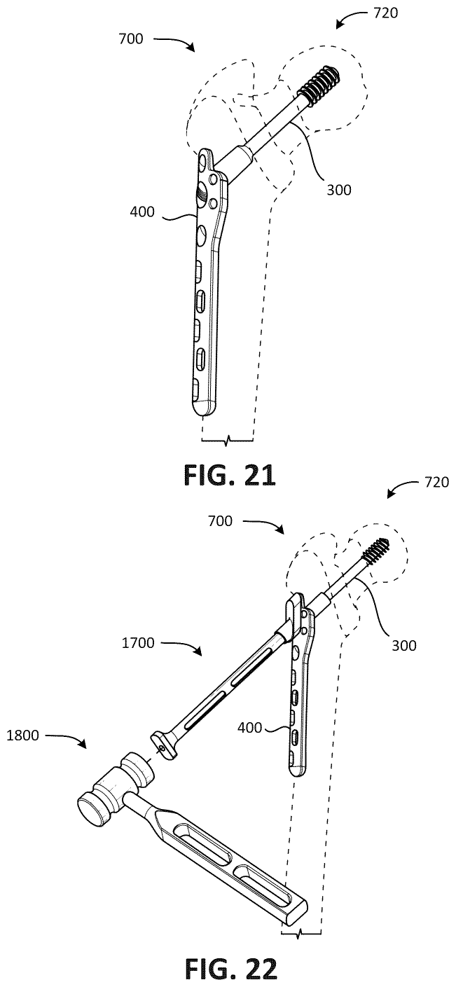

FIG. 21 illustrates a side view the femoral support member 400, the femoral fastener 300, and the femur 700 after the inserter tool 1600 has been removed.

FIG. 22 illustrates a side view of an impactor 1700 and a mallet 1800 that may be utilized to further seat the femoral support member 400 against the femur 700, in some embodiments.

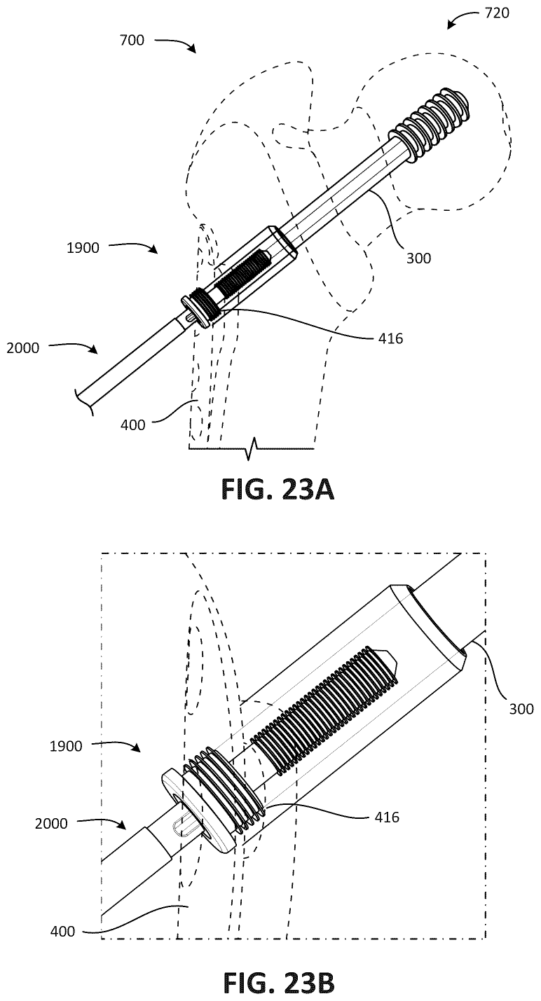

FIG. 23A illustrates a side view of a compression screw 1900 being inserted into the femoral support member 400 and the femoral fastener 300 with a driver tool 2000, according to an embodiment of the present disclosure. FIG. 23B illustrates a close-up view of the compression screw 1900 shown in FIG. 23A. If desired, the compression screw 1900 may be utilized to generate a compression force across a bone fracture or fracture 725 of the femur 700 by threading the compression screw 1900 into the femoral fastener 300 while a head of the compression screw 1900 presses against the barrel shoulder 416 in the passageway 410. The compression screw 1900 may be rotated until a desired compression force is achieved. To maintain the compression force, one or more support fasteners 2800 (e.g., see FIG. 29) may then be placed across the fracture while the compression screw 1900 is generating the compression force. The compression screw 1900 may then be removed after compression has been achieved and maintained by the one or more support fasteners 2800. However, in some embodiments the compression screw 1900 may left inside the femoral fixation assembly 600.

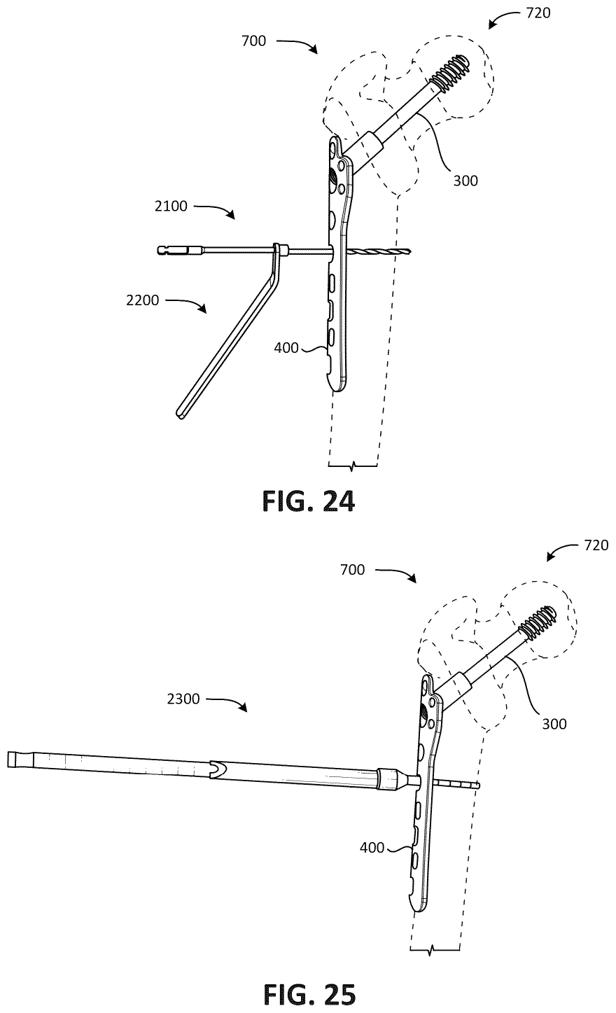

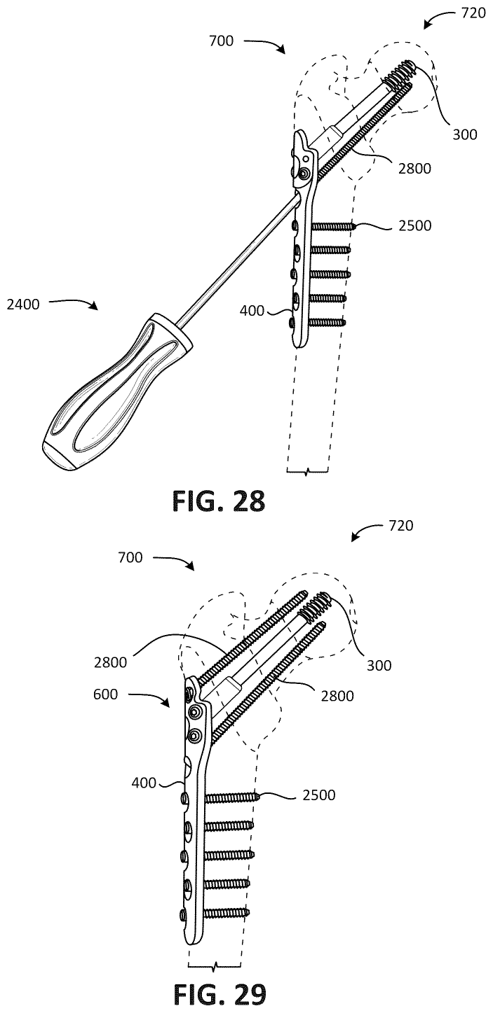

FIGS. 24-29 illustrate the installation of various bone screws to affix the femoral fixation assembly 600 to the femur 700. Specifically, FIG. 24 illustrates a side view of a drill bit 2100 and a drill bit guide 2200 forming one or more bone tunnels in the femur 700; FIG. 25 illustrates a side view of a bone tunnel depth gauge 2300 measuring a depth of a bone tunnel to ascertain a required length for a bone screw; FIG. 26 illustrates a side view of a driver tool 2400 installing one or more bone plate fasteners 2500 into the femur 700; FIG. 27 illustrates a side view of a drill bit 2600 and drill bit guide 2700 forming a bone tunnel into the head 720 of the femur 700; FIG. 28 illustrates a side view of a driver tool 2400 installing one or more support fasteners 2800 into the head 720 of the femur 700; and FIG. 29 illustrates a side view of the femoral fixation assembly 600 after the one or more support fasteners 2800 have been installed.

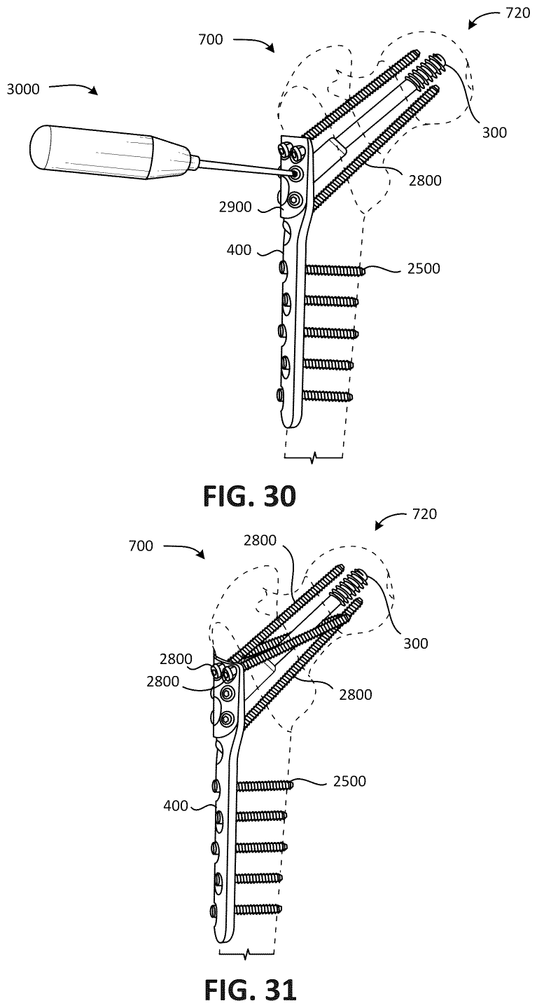

FIG. 30 illustrates a side view of an extension plate 2900 being coupled to the femoral support member 400 with a driver tool 3000, according to some embodiments of the present disclosure. FIG. 31 illustrates a side view of the femoral fixation assembly 600 after the extension plate 2900 has been coupled to the femoral support member 400 and support fasteners 2800 have been inserted into the head 720 of the femur 700 through the extension plate 2900.

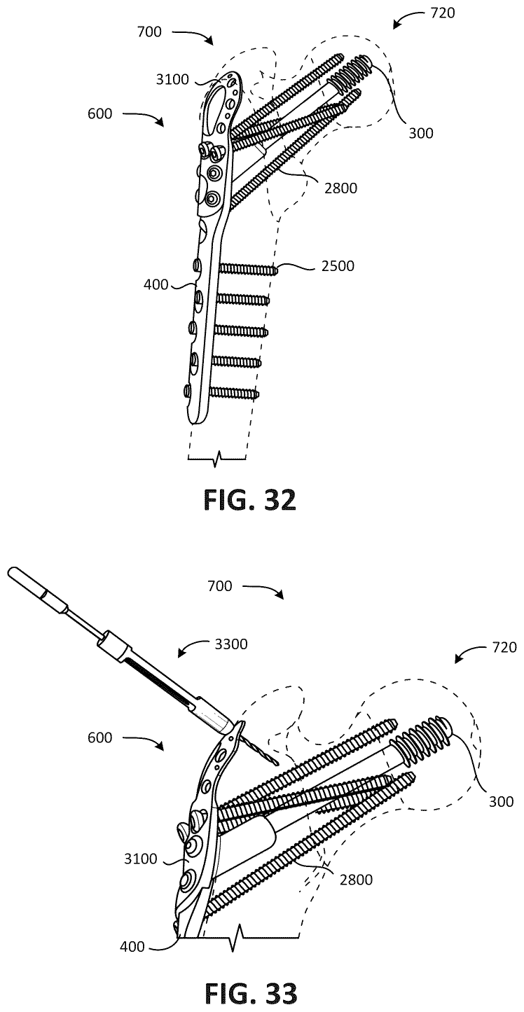

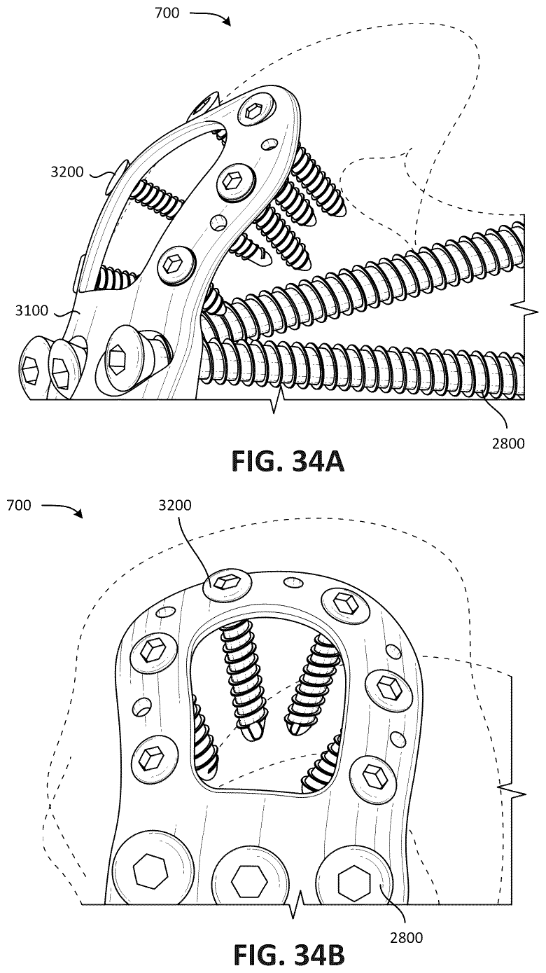

FIGS. 32-34B illustrate installation of a trochanter plate 3100 to the femur 700, according to some embodiments of the present disclosure. Specifically, FIG. 32 illustrates a side view of the femoral fixation assembly 600 after a trochanter plate 3100 has been coupled to the femoral support member 400 and support fasteners 2800 have been inserted into the head 720 of the femur 700 through the trochanter plate 3100; FIG. 33 illustrates a side view of the femoral fixation assembly 600 with a drill bit 3300 forming one or more bone tunnels in the femur 700 to receive one or more trochanter plate fasteners 3200 to secure the trochanter plate 3100 to the femur 700; FIG. 34A illustrates a perspective side view of the femoral fixation assembly 600 after the one or more trochanter plate fasteners 3200 have been installed into the femur 700 through the trochanter plate 3100; and FIG. 34B illustrates a front view of the femoral fixation assembly 600 of FIG. 34A.

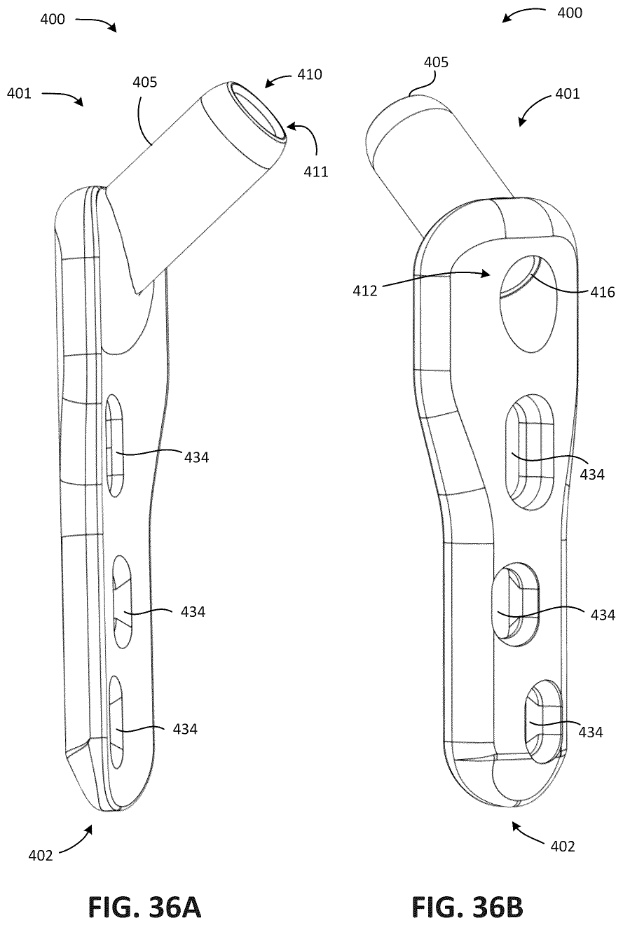

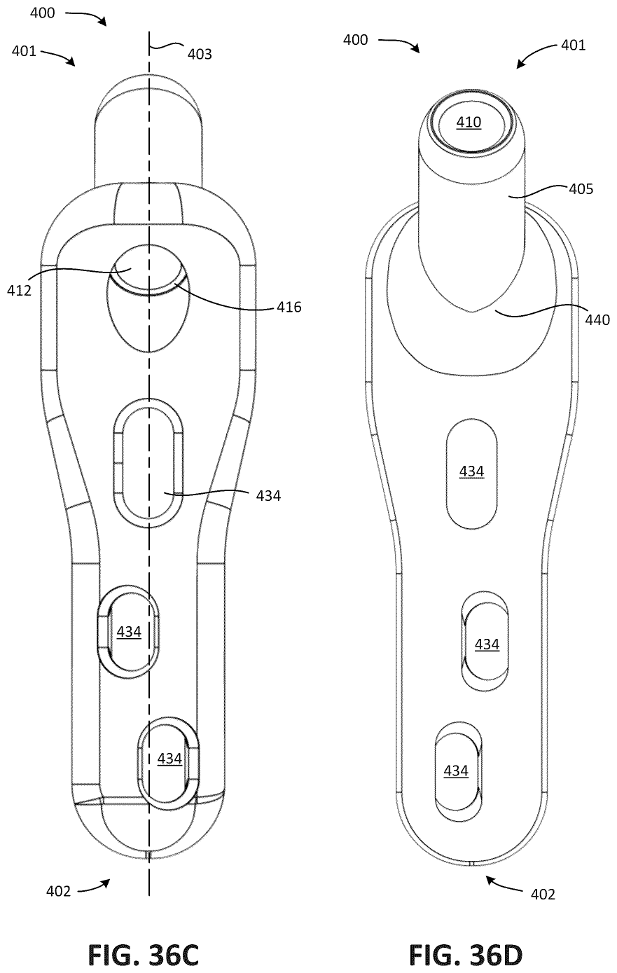

FIGS. 36A-D illustrate various views of a femoral support member 400, according to another embodiment of the present disclosure. Specifically, FIG. 36A is a perspective side view of the femoral support member 400, FIG. 36B is another perspective side view of the femoral support member 400, FIG. 36C is a front view of the femoral support member 400, and FIG. 36D is a rear view of the femoral support member 400.

In general, the femoral support member 400 may include an elongate body having a proximal end 401, a distal end 402, a longitudinal axis 403, and a barrel 405.

In some embodiments, the femoral support member 400 may comprise a bone plate configured to engage the lateral surface 705 of a femoral bone.

In some embodiments, the femoral support member 400 may comprise an intramedullary nail (not shown) configured for implantation within an intramedullary canal of a femoral bone.

In some embodiments, the femoral support member 400 may include one or more bone plate apertures 434 configured to receive one or more bone plate fasteners 2500 therethrough.

In some embodiments, the barrel 405 may include a passageway 410 formed therethrough having a first opening 411 and a second opening 412 opposite the first opening 411.

In some embodiments, the barrel 405 and/or the passageway 410 may be formed through the femoral support member 400 with a longitudinal axis 408 at an angle 409 with respect to the longitudinal axis 403 of the femoral support member 400, as previously described herein.

In some embodiments, the angle 409 of the barrel 405 and/or the passageway 410 with respect to the longitudinal axis 403 of the femoral support member 400 may be an acute angle. However, it will also be understood that in some embodiments the angle 409 may be a right angle and/or an obtuse angle.

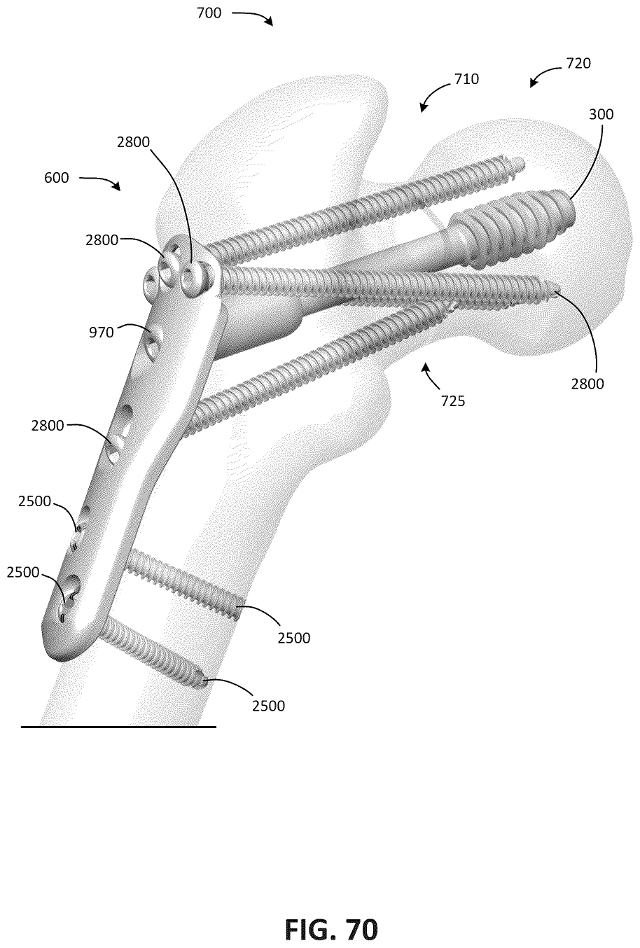

In some embodiments, the passageway 410 may include a barrel shoulder 416 positioned near the second opening 412. A compression fastener 970 may be utilized with the barrel shoulder 416 to generate a compression force across the fracture 725 by threadingly engaging the compression fastener 970 with the internal thread 380 formed within the longitudinal passageway 390 of the femoral fastener 300, while a head of the compression fastener 970 presses against the barrel shoulder 416 within in the passageway 410. The compression fastener 970 may be rotated until a desired compression force is achieved. To maintain the compression force, one or more support fasteners 2800 (e.g., see FIG. 70) may be placed across the fracture 725 while the compression fastener 970 is generating the compression force. The compression fastener 970 may be removed after compression has been achieved and maintained by the one or more support fasteners 2800. However, in some embodiments the compression fastener 970 may left inside the femoral fixation assembly 600.

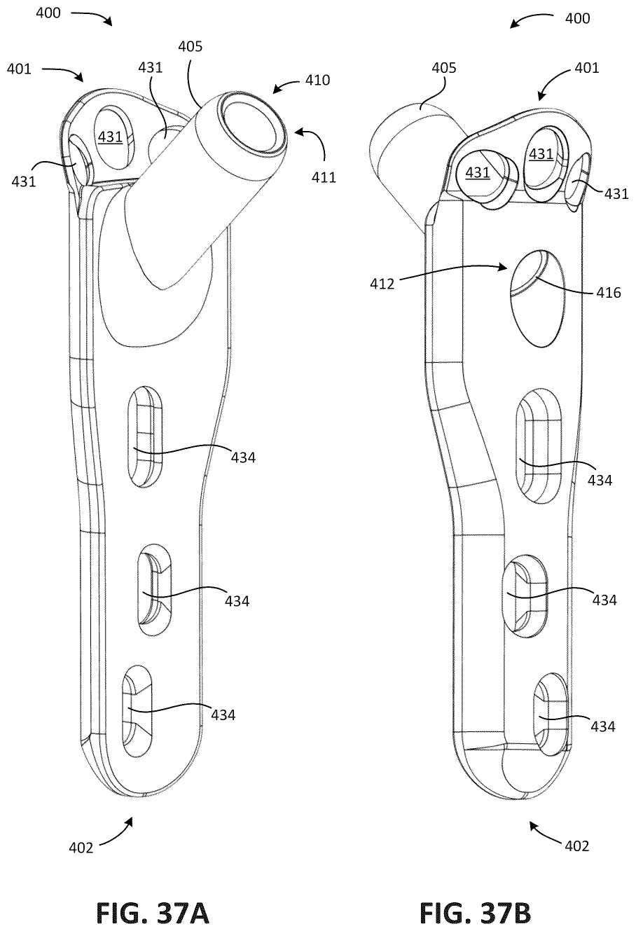

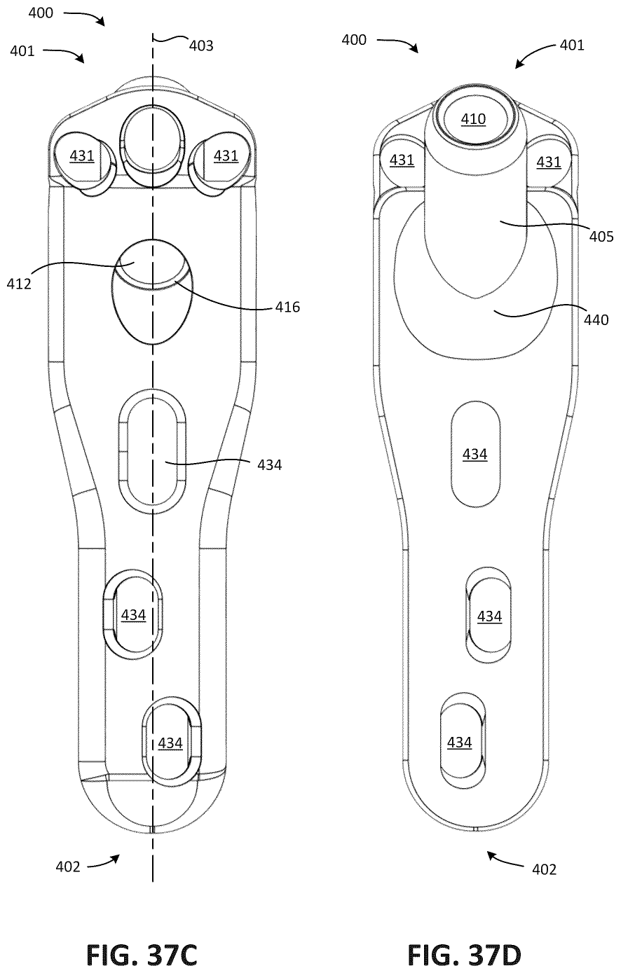

FIGS. 37A-D illustrate various views of a femoral support member 400, according to another embodiment of the present disclosure. Specifically, FIG. 37A is a perspective side view of the femoral support member 400, FIG. 37B is another perspective side view of the femoral support member 400, FIG. 37C is a front view of the femoral support member 400, and FIG. 37D is a rear view of the femoral support member 400.

In general, the femoral support member 400 may include an elongate body having a proximal end 401, a distal end 402, a longitudinal axis 403, and a barrel 405.

In some embodiments, the femoral support member 400 may comprise a bone plate configured to engage the lateral surface 705 of a femoral bone.

In some embodiments, the femoral support member 400 may comprise an intramedullary nail (not shown) configured for implantation within an intramedullary canal of a femoral bone.

In some embodiments, the femoral support member 400 may include one or more bone plate apertures 434 configured to receive one or more bone plate fasteners 2500 therethrough and/or one or more superior support apertures 431 configured to receive one or more support fasteners 2800 therethrough.

In some embodiments, the barrel 405 may include a passageway 410 formed therethrough having a first opening 411 and a second opening 412 opposite the first opening 411.

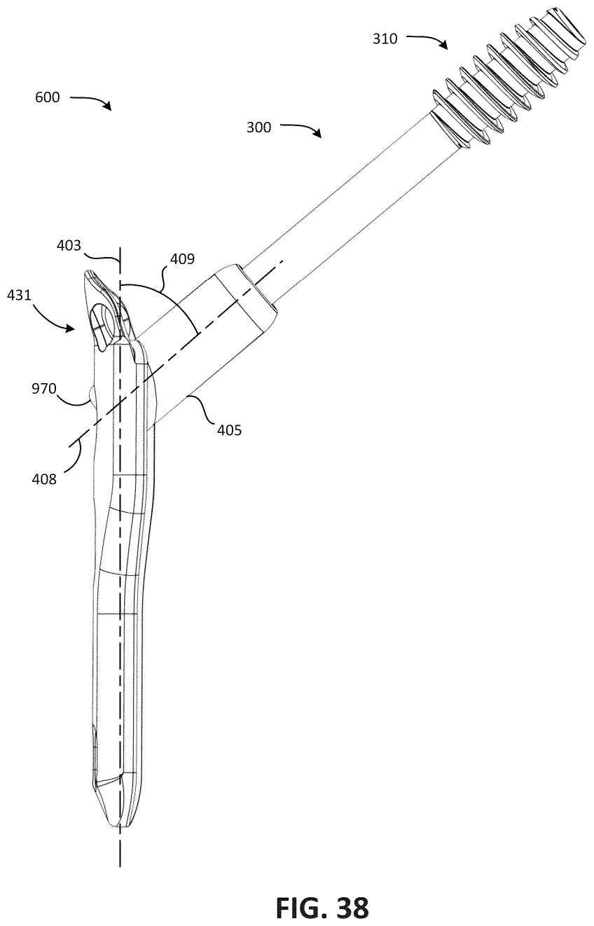

In some embodiments, the barrel 405 and/or the passageway 410 may be formed through the femoral support member 400 with a longitudinal axis 408 at an angle 409 with respect to the longitudinal axis 403 of the femoral support member 400. (e.g., see FIGS. 7 and 38).

In some embodiments, the angle 409 of the barrel 405 and/or the passageway 410 with respect to the longitudinal axis 403 of the femoral support member 400 may be an acute angle. However, it will also be understood that in some embodiments the angle 409 may be a right angle and/or an obtuse angle.

In some embodiments, the passageway 410 may include a barrel shoulder 416 positioned near the second opening 412. The compression fastener 970 may be utilized with the barrel shoulder 416 to generate a compression force across the fracture 725, as previously described.

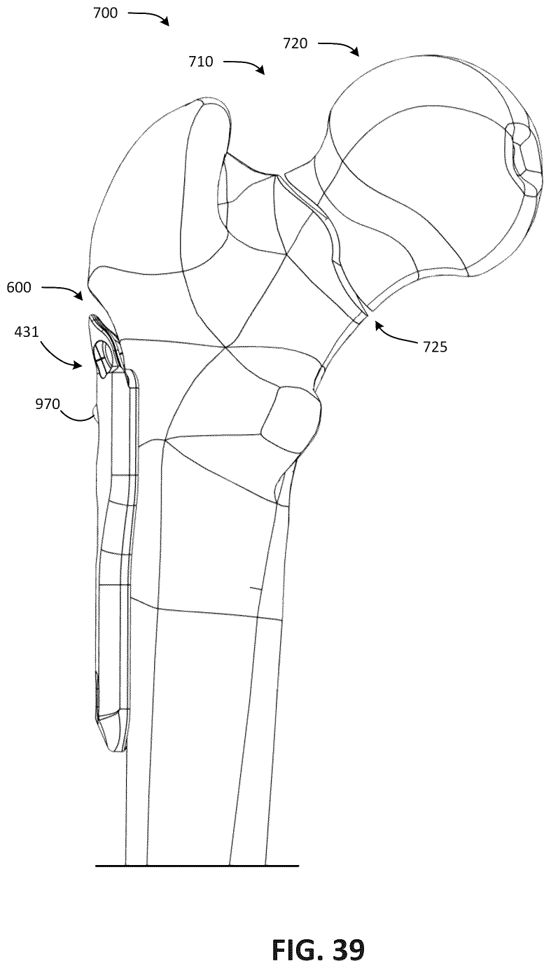

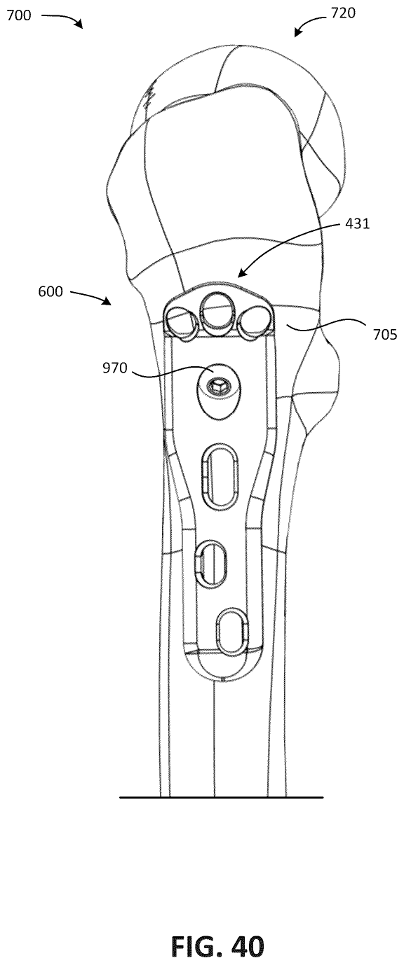

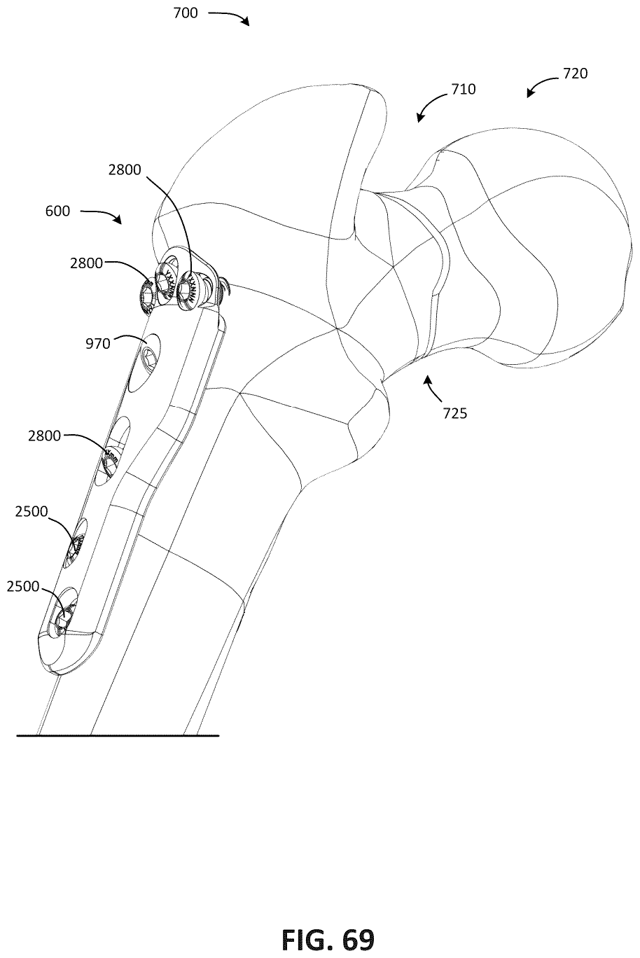

FIGS. 38-40, 69, and 70 illustrate various views of a femoral fixation assembly 600 comprising the femoral support member 400 of FIGS. 37A-37D (or FIGS. 36A-36D) in combination with the femoral fastener 300 of FIGS. 3A-3D, according to embodiments of the present disclosure. Specifically, FIG. 38 is a side view of the femoral fixation assembly 600; FIG. 39 is a side view of the femoral fixation assembly 600 implanted within the femur 700; FIG. 40 is a front view of the femoral fixation assembly of FIG. 39; FIG. 69 is a perspective view of the femoral fixation assembly 600 installed within the femur 700, along with various bone fasteners; and FIG. 70 is another perspective view of the femoral fixation assembly 600 from FIG. 69 that shows the femoral fastener 300 and one or more support fasteners 2800 extending into the head 720 of the femur 700, as well as one or more bone plate fasteners 2500 extending into the shaft of the femur 700.

When the femoral fastener 300 is implanted within the neck 710 and the head 720 of the femur 700, and the femoral support member 400 is oriented with respect to a longitudinal axis of the femoral bone, at least a portion of the shaft 305 of the femoral fastener 300 may be slidingly received within the passageway 410 of the barrel 405 through the first opening 411 of the passageway 410. In this manner, the femoral fastener 300 may be allowed to slide further into the passageway 410 of the barrel 405 as the femur 700 collapses due to the bone remodeling process, the healing process, continued use of the femur 700 over time, etc.

In some embodiments, the femoral support member 400 may (or may not) include the stop member 500 that is configured to limit an amount of collapse that the femur 700 may be permitted to undergo, as previously described herein.

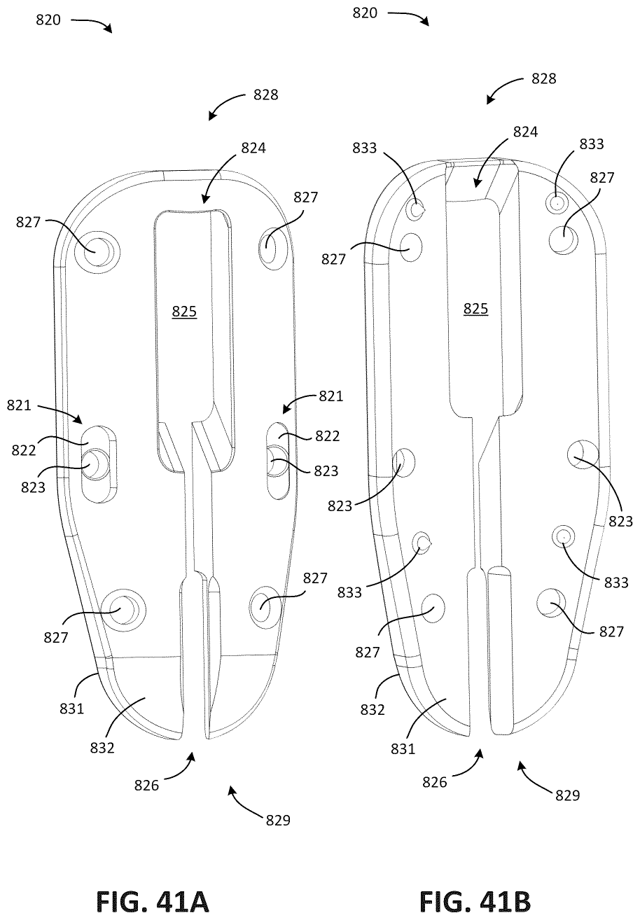

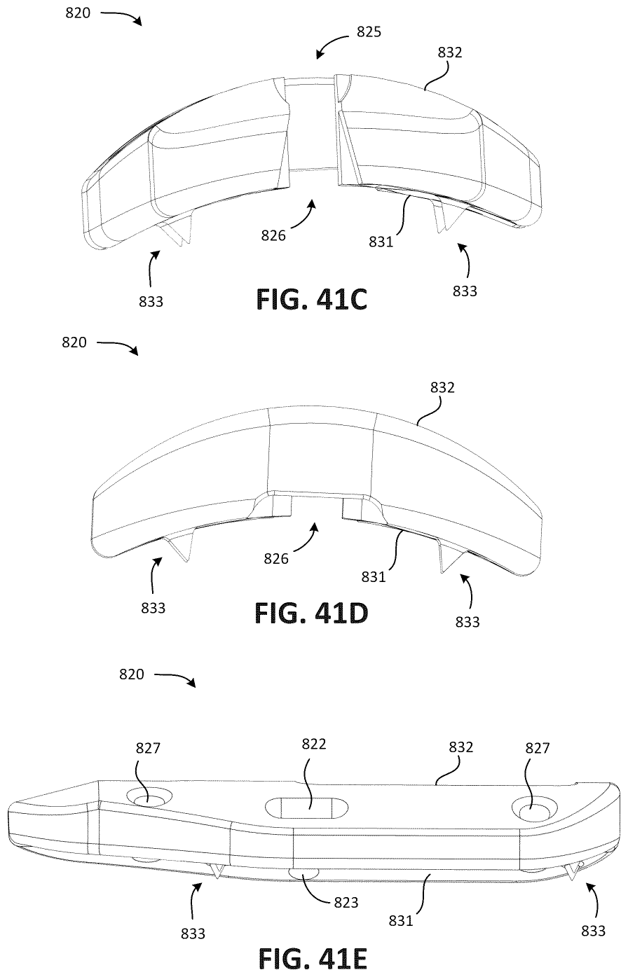

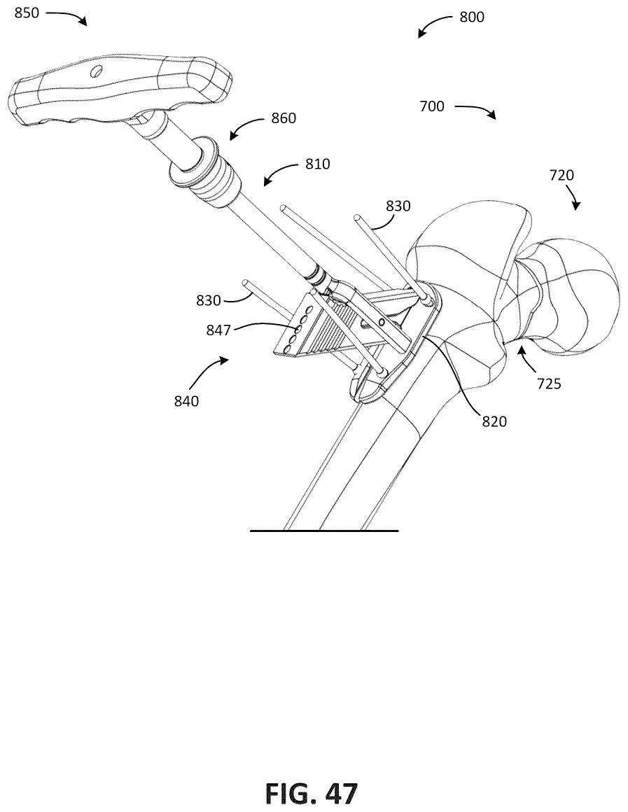

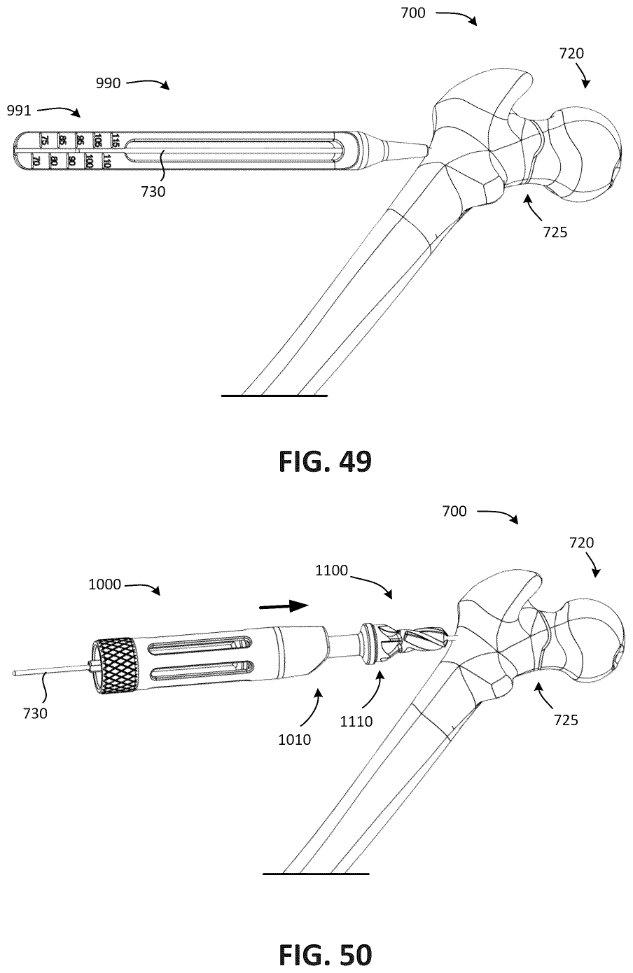

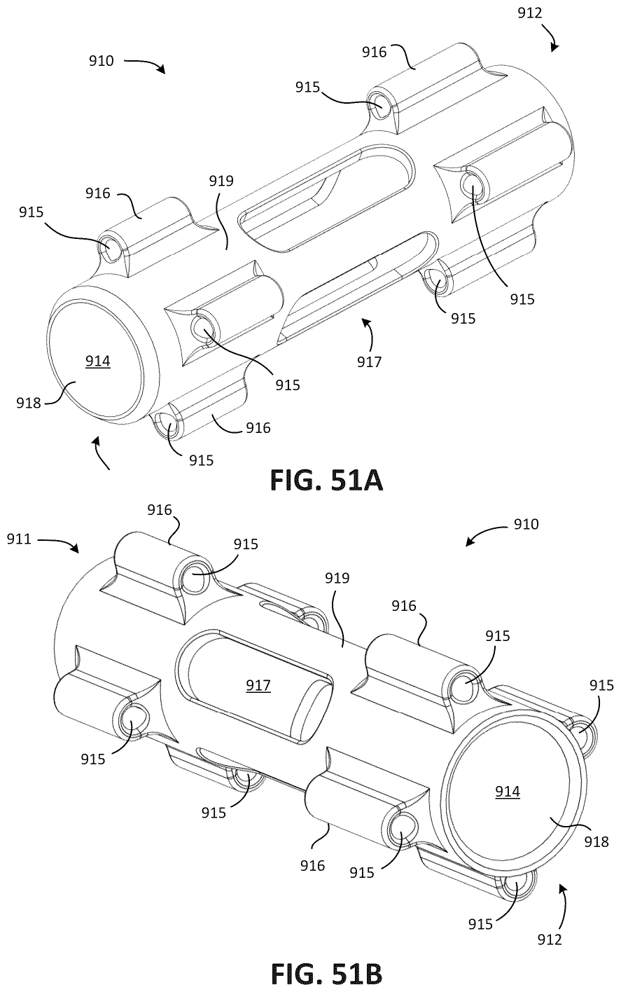

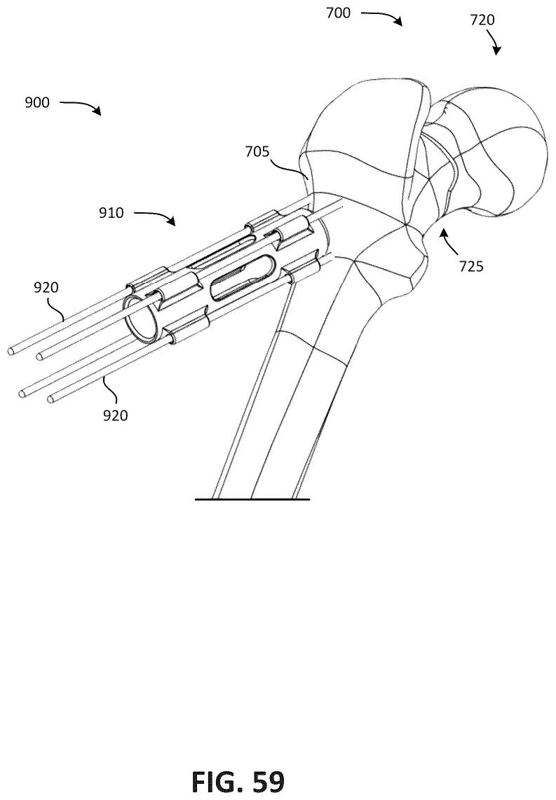

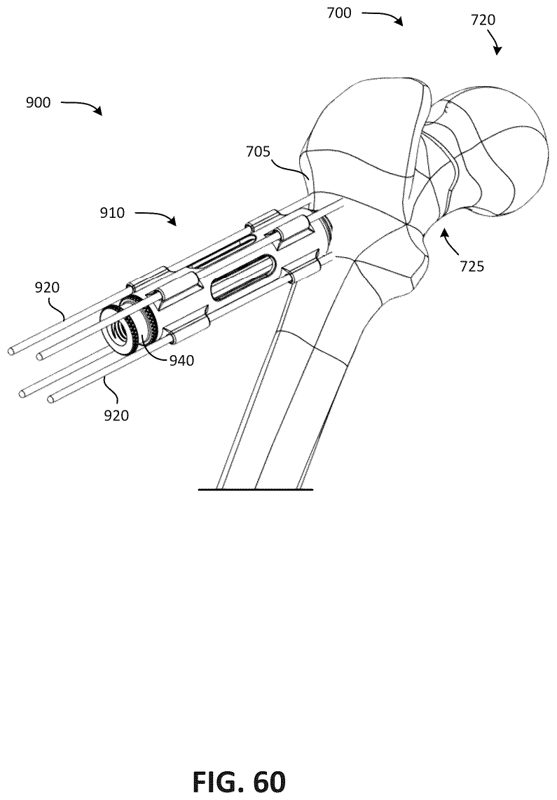

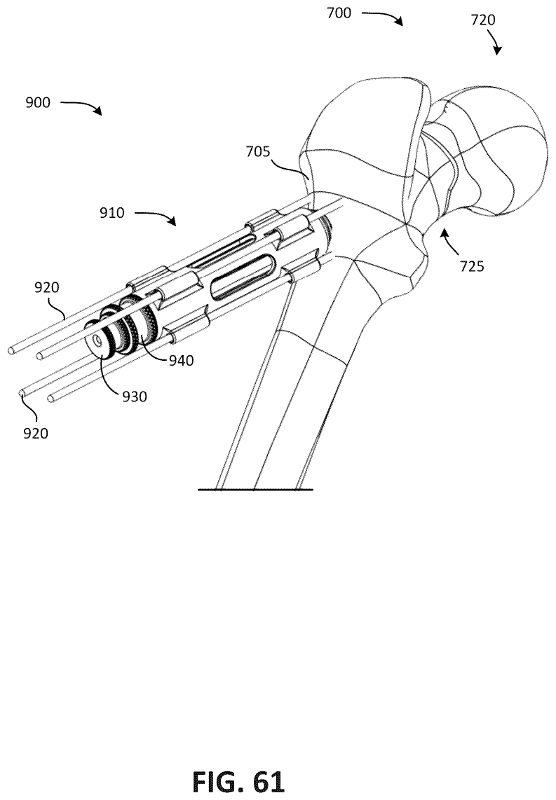

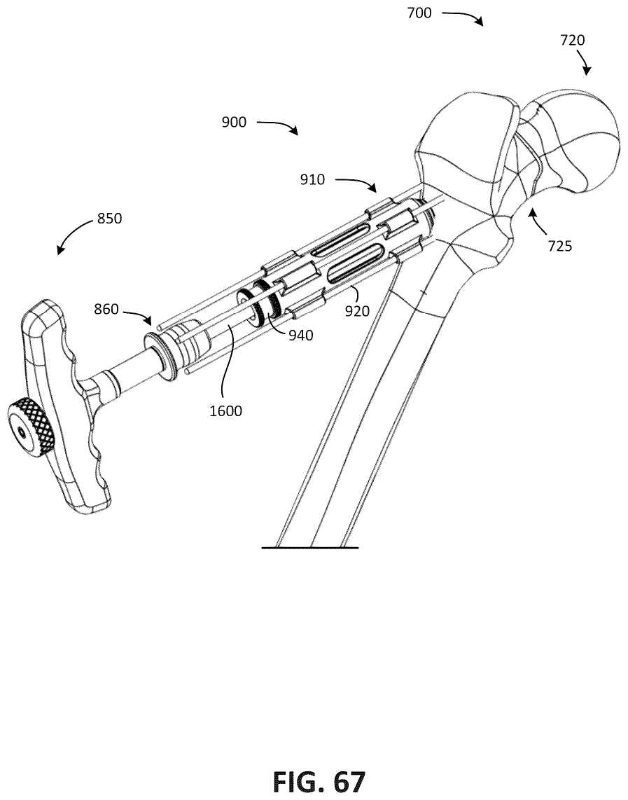

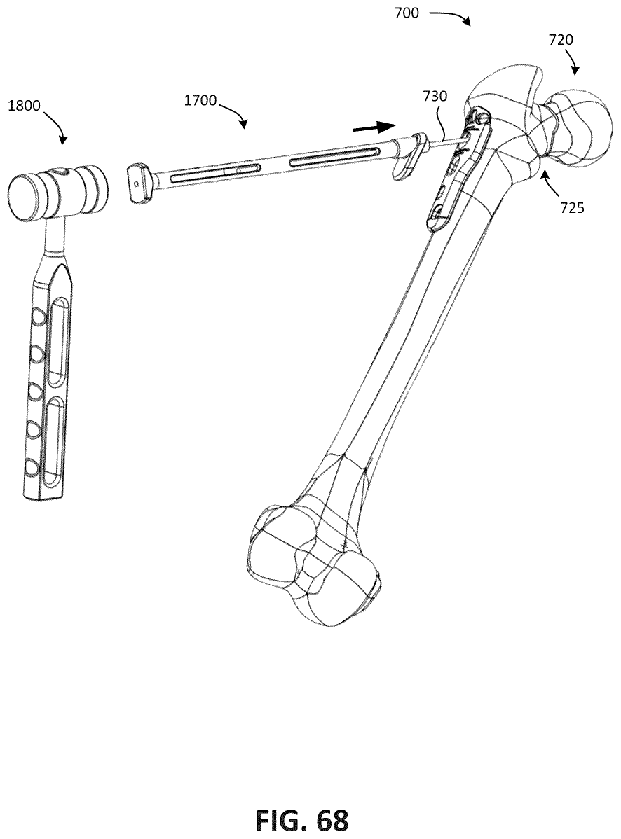

FIGS. 41A-70 illustrate various views of surgical instruments and procedures that may be utilized to install the femoral fixation assembly 600 into the neck 710 and head 720 of the femur 700. Specifically, FIGS. 41A-48A illustrate various views of a targeting guide assembly 800 and its method of use for installing the guide pin 730 into the neck 710 and head 720 of the femur 700; FIGS. 49 and 50 respectively illustrate a guide pin depth gauge measuring step and a reaming step; FIGS. 51A-67 illustrate various views of an anti-rotation assembly and its method of use for installing the femoral fastener 300 into the neck 710 and head 720 of the femur 700; and FIGS. 68-70 illustrate various method steps that may be utilized to affix the femoral support member 400 to the femur 700 once the femoral fastener 300 has been installed within the neck 710 and head 720 of the femur 700.

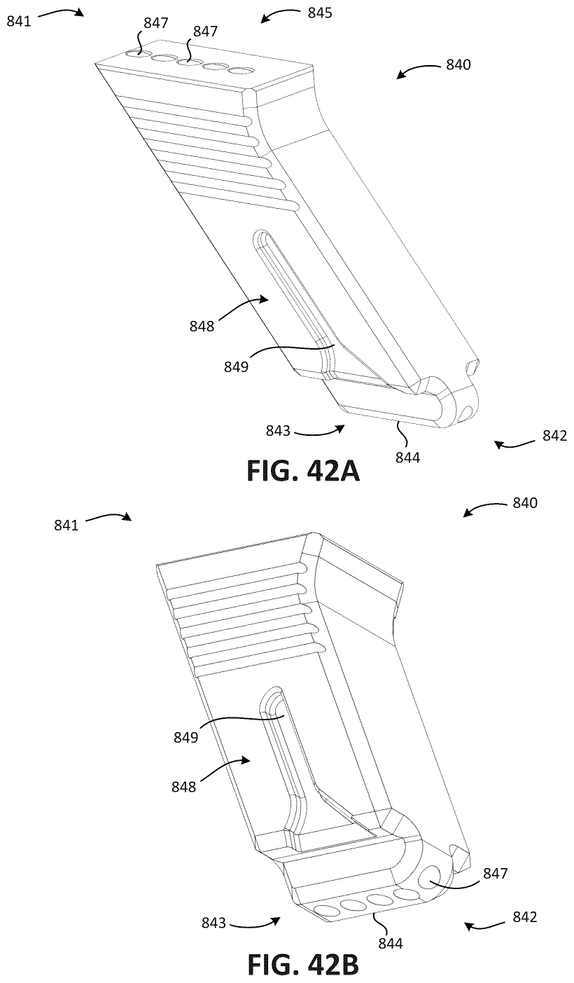

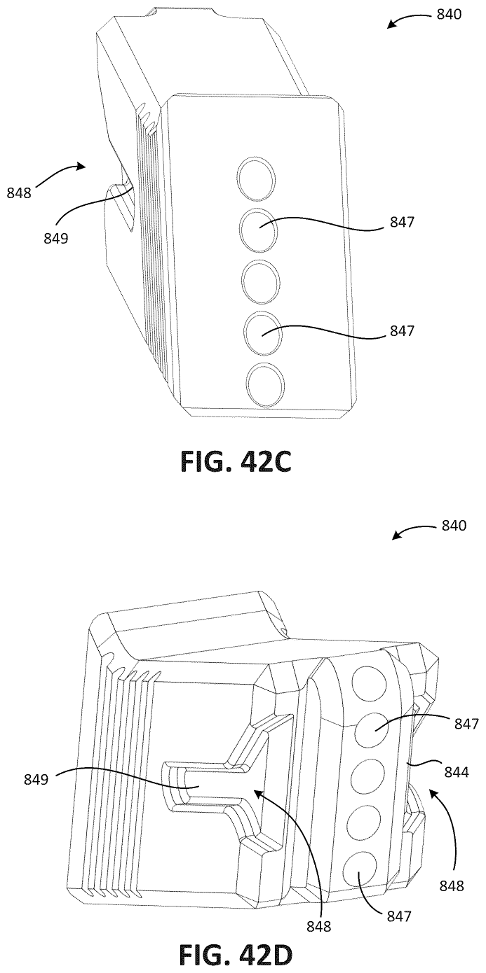

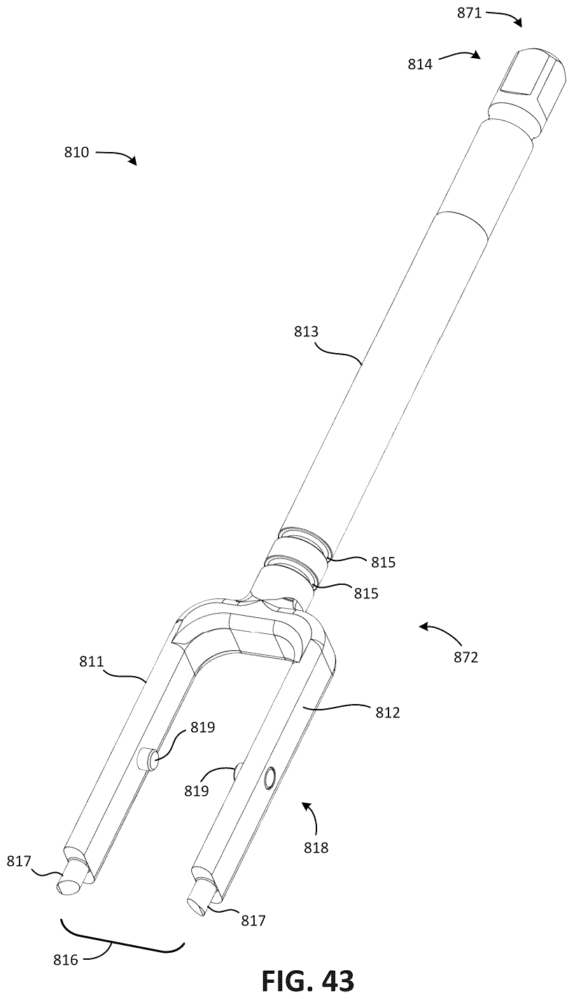

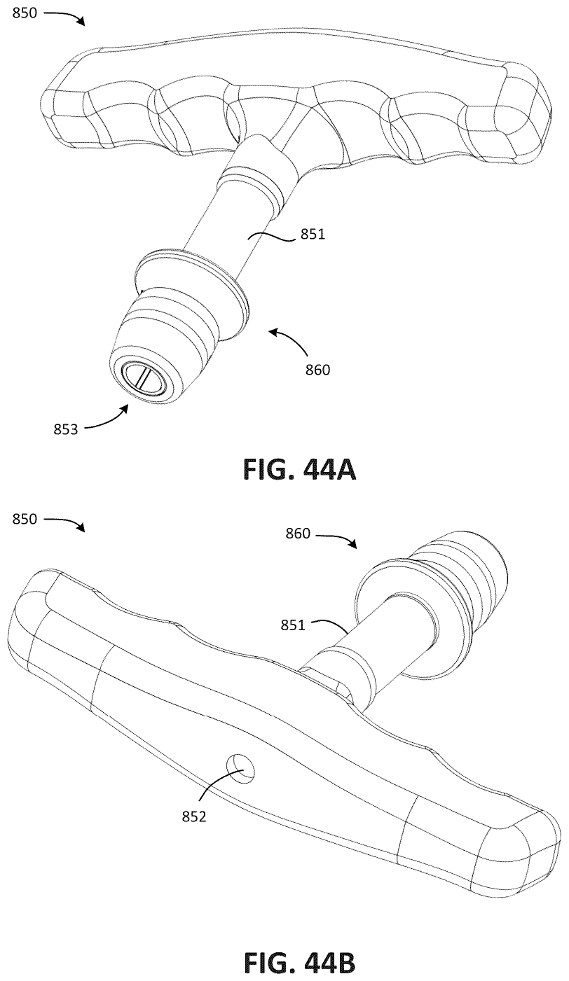

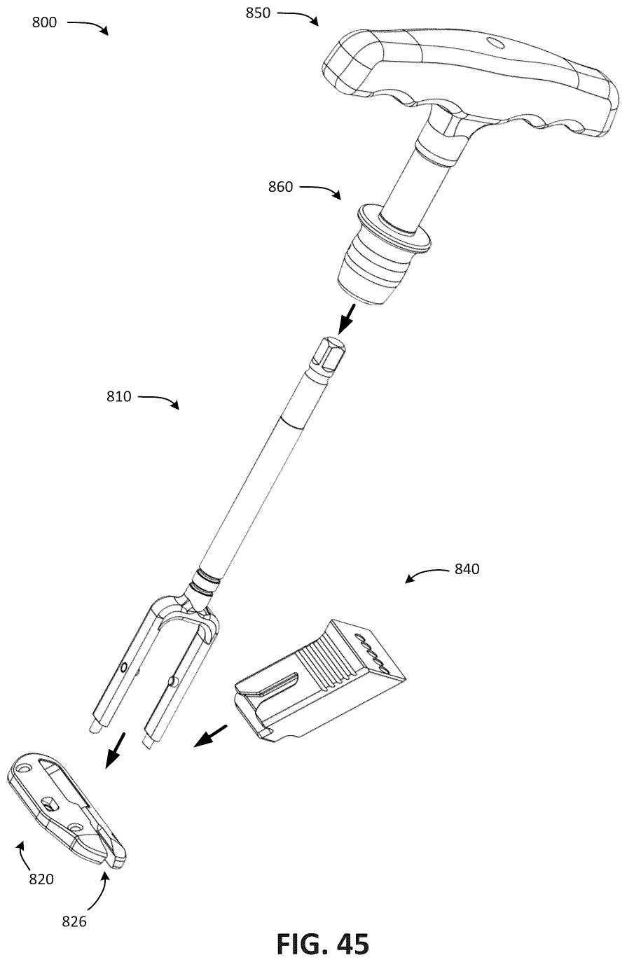

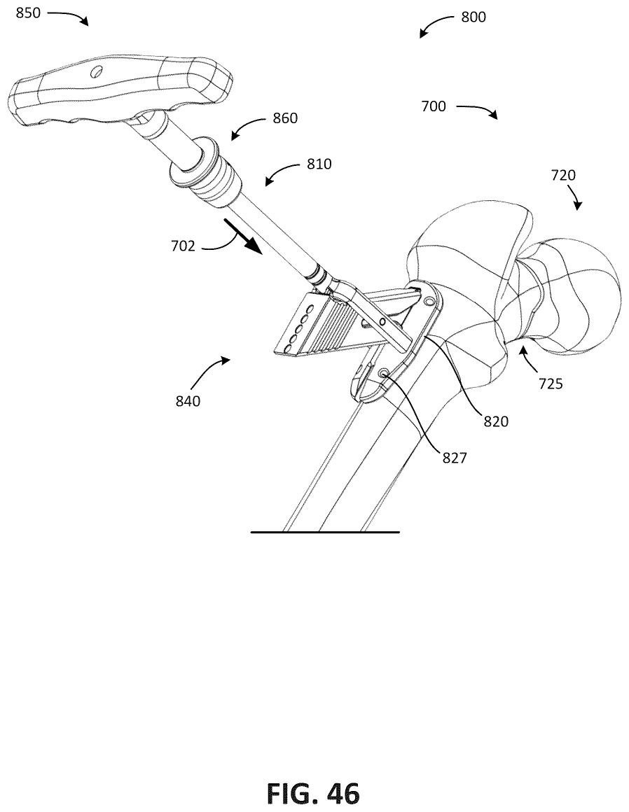

FIGS. 41A-48B illustrate various views of a targeting guide assembly 800 and its method of use for installing the guide pin 730 into the neck 710 and head 720 of the femur 700. Specifically, FIGS. 41A-41E show various views of a targeting guide base 820, according to another embodiment of the present disclosure; FIGS. 42A-42D show various views of a targeting guide insert 840, according to another embodiment of the present disclosure; FIG. 43 shows the targeting guide positioner 810 of FIG. 11; FIGS. 44A-44B show various views of the handle 850 and the adjustable insert retainer 860 of FIG. 11; FIG. 45 shows an exploded view of the targeting guide assembly 800; and FIGS. 46-48B show the targeting guide assembly 800 being assembled onto a femur 700 to place the guide pin 730 into the neck 710 and the head 720 of the femur 700.

With reference to FIGS. 41A-41E, the targeting guide base 820 may generally include a superior end 828, an inferior end 829, a bone-facing surface 831, an opposing surface 832 opposite the bone-facing surface 831, a locating slot 825 formed through the targeting guide base 820 between the bone-facing surface 831 and the opposing surface 832, and a removal slot 826 formed through the targeting guide base 820 between the bone-facing surface 831 and the opposing surface 832. The removal slot 826 may extend from the inferior end 829 of the targeting guide base 820 to the locating slot 825.

In some embodiments, the bone-facing surface 831 may comprise a concave shape the may be shaped to engage the lateral surface 705 of the femur 700. In some embodiments, the opposing surface 832 may comprise a convex shape. However, it will be understood that the bone-facing surface 831 and/or the opposing surface 832 may comprise any shape or shapes (e.g., straight, concave, convex, etc.).

In some embodiments, the targeting guide base 820 may also include one or more placement pin holes 827 configured to receive one or more placement pins 830 (e.g., olive pins, etc.) therethrough to couple the targeting guide base 820 to the femur 700. In some embodiments, the one or more placement pin holes 827 may include chamfered surfaces configured to accept the olive portions of olive pins thereon to secure the targeting guide base 820 to the lateral surface 705 of the femur 700 (e.g., see FIGS. 11 and 47-48B).

In some embodiments, the targeting guide base 820 may also include one or more bone fixation members 833 (e.g., spikes, teeth, projections, etc.) configured to stabilize the targeting guide base 820 against the femur 700.

In some embodiments, the targeting guide base 820 may also include a base positioner connection feature 821 configured to removably couple the targeting guide base 820 to the targeting guide positioner 810. For example, in some embodiments the base positioner connection feature 821 may include one or more indentations 822 and/or one or more holes 823 formed in the targeting guide base 820 that are configured to removably couple the targeting guide base 820 to the targeting guide positioner 810. However, it will be understood that any structure(s) may be utilized to removably couple the targeting guide base 820 to the targeting guide positioner 810. Moreover, in at least some embodiments the targeting guide base 820 may be integrally/monolithically formed with the targeting guide positioner 810, or otherwise permanently, or semi-permanently, coupled with each other (i.e., welded together, glued together, etc.).

In some embodiments, the targeting guide base 820 may also include a base insert connection feature 824 configured to removably couple the targeting guide base 820 to the targeting guide insert 840. For example, in some embodiments the base insert connection feature 824 may include all (or a portion) of the locating slot 825. However, it will be understood that any structure(s) may be utilized to removably couple the targeting guide base 820 to the targeting guide insert 840, as will be discussed below in more detail with respect to the targeting guide insert 840.

In some embodiments, the locating slot 825 may be angled superiorly, as shown in FIGS. 41A and 41B. In some embodiments, the locating slot 825 may be angled superiorly to match the angle 409 of the barrel 405 with respect to the longitudinal axis 403 of the plate or femoral support member 400. However, it will be understood that the locating slot 825 may comprise any shape(s) or angles (or no angle at all).

In some embodiments, the removal slot 826 may be wider toward the inferior end 829 of the targeting guide base 820, and narrower toward the locating slot 825. However, it will be understood that the locating slot 825 may comprise any shape or shapes.

The targeting guide base 820 configurations/shapes shown in FIGS. 11, 35, and 41A-41E, which include the removal slot 826, allow for smaller incisions (i.e., more minimally invasive incisions) to be utilized during the implantation procedure by enabling insertion/removal of the targeting guide base 820 from the superior direction 704 at the surgical site to engage and/or disengage with the lateral surface 705 of the femur 700. Moreover, the inclusion of the removal slot 826 also allows for larger targeting guide bases to be utilized (e.g., targeting guide bases that mimic the actual size of the femoral support member 400 to be installed on the femur 700 to help visualize the final location of the femoral support member 400), while maintaining this smaller, more minimally invasive, incision technique.

With reference to FIGS. 42A-42D, the targeting guide insert 840 may generally include a proximal end 841, a distal end 842, at least one insert passageway 847 formed through the targeting guide insert 840 between the proximal end 841 and the distal end 842 of the targeting guide insert 840, and an insert base connection feature 843 at the distal end 842 of the targeting guide insert 840.

In some embodiments, the insert base connection feature 843 may comprise an insert projection 844 configured to be at least partially received within the locating slot 825 of the targeting guide base 820 to removably couple the targeting guide insert 840 to the targeting guide base 820 at the locating slot 825. However, it will be understood that the insert base connection feature 843 and/or the base insert connection feature 824 may include any structure(s) for removably coupling the targeting guide insert 840 to the targeting guide base 820 (e.g., an attachment screw/threaded hole structure, a mating ball/detent structure, a quick release structure, etc.). In this manner, different targeting guide inserts with different configurations may be easily swapped out with each other during the procedure without removing the targeting guide base 820.

In some embodiments, the at least one insert passageway 847 of the targeting guide insert 840 may be configured to orient the guide pin 730 placed therethrough into the neck 710 and the head 720 of the femur 700 to guide placement of the femoral fastener 300 therein.

In some embodiments, the at least one insert passageway 847 of the targeting guide insert 840 may comprises a plurality of insert passageways formed through the targeting guide insert 840. In some embodiments, the plurality of insert passageways may be arranged in a linear fashion (e.g., superiorly-inferiorly) to allow for superior-inferior adjustability for targeting the guide pin 730 into the head 720 of the femur 700, without the need of removing/adjusting the targeting guide insert 840 position relative to the targeting guide base 820.

In some embodiments, the targeting guide insert 840 may comprise a fixed targeting guide insert to orient the guide pin 730 along a single direction into the head 720 of the femur 700. For example, the at least one insert passageway 847 may comprise a straight or cylindrical shape.

In some embodiments, the targeting guide insert 840 may comprise a variable targeting guide insert to allow for orienting the guide pin 730 along a plurality of directions into the head 720 of the femur 700. For example, the at least one insert passageway 847 may comprise a conical shape, an oval shape, a flared shape, a tapered shape, etc. (e.g., see FIG. 12 showing the interior space 845 and the adjustable angle 846 previously discussed herein). In this manner, the variable targeting guide insert may allow for anterior-posterior (and/or superior-inferior) adjustability for targeting the guide pin 730 into the head 720 of the femur 700.

In some embodiments, the variable targeting guide insert may allow for infinite adjustability for targeting the guide pin 730 into the head 720 of the femur 700.

In some embodiments, the variable targeting guide insert may allow for discrete adjustability for targeting the guide pin 730 into the head 720 of the femur 700.

In some embodiments, the targeting guide insert 840 may comprise any combination of fixed and/or variable insert passageways to allow for any number or combinations of orientation(s) to guide the guide pin 730 into the head 720 of the femur 700.

In some embodiments, the targeting guide insert 840 may also include an insert positioner connection feature 848 configured to removably couple the targeting guide insert 840 to the targeting guide positioner 810, as will be discussed below in more detail. In some embodiments, the insert positioner connection feature 848 may include one or more insert slots 849 configured to removably couple the targeting guide insert 840 to the targeting guide positioner 810.

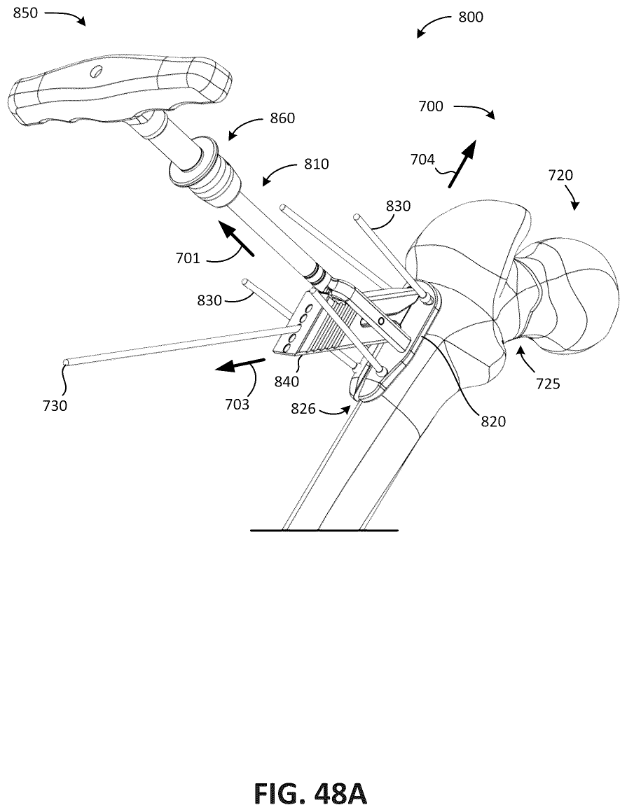

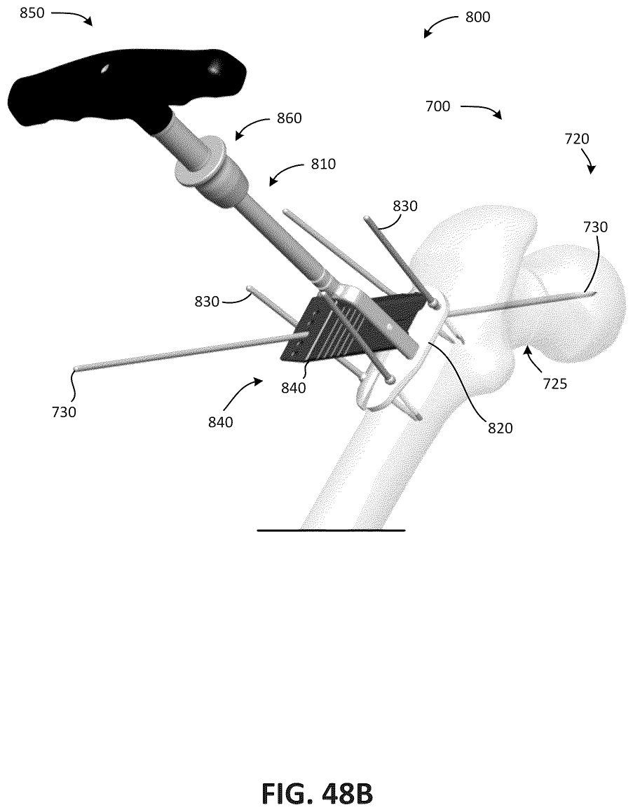

In some embodiments, once the guide pin 730 has been placed into the neck 710 and the head 720 of the femur 700, the targeting guide insert 840 may be configured to detach from the targeting guide base 820 and slidingly disengage from the guide pin 730 along a proximal direction 703 (e.g., see FIG. 48A). The targeting guide base 820 may then be movable along a superior direction 704 to release the guide pin 730 inferiorly through the removal slot 826 that extends from the locating slot 825 to the inferior end 829 of the targeting guide base 820, as previously discussed herein.

In some embodiments, the targeting guide assembly 800 may also include the targeting guide positioner 810 shown in FIG. 43. The targeting guide positioner 810 may generally include a positioner shaft 813, a positioner first arm 811 projecting from a distal end 872 of the positioner shaft 813, a positioner second arm 812 projecting from the distal end 872 of the positioner shaft 813, opposite the positioner first arm 811, a positioner base connection feature 816 configured to couple the targeting guide positioner 810 to the targeting guide base 820, and at least one positioner insert connection feature 818 configured to couple the targeting guide positioner 810 to the targeting guide insert 840.

In some embodiments, the positioner base connection feature 816 may include one or more prongs 817 extending from the positioner first arm 811 and/or the positioner second arm 812 and configured to engage the one or more indentations 822 and/or the one or more holes 823 formed in the targeting guide base 820 to removably couple the targeting guide positioner 810 with the targeting guide base 820.

In some embodiments, the positioner insert connection feature 818 may include one or more pins 819 configured to engage the one or more insert slots 849 of the targeting guide insert 840 to removably couple the targeting guide positioner 810 with the targeting guide insert 840.

In some embodiments, the targeting guide positioner 810 may also include a positioner torque connection feature 814 at the proximal end 871 of the positioner shaft 813, as well as one or more shaft grooves 815 formed in the distal end 872 of the positioner shaft 813.

In some embodiments, the targeting guide assembly 800 may also include the handle 850 and/or the adjustable insert retainer 860 that are shown in FIGS. 11 and 44A-48B. The handle 850 may generally include a handle shaft 851, a handle passageway 852 formed through the handle shaft 851, and a handle torque connection feature 853 configured to receive a corresponding torque connection feature therein from one or more of the various tools that are described herein (e.g., the targeting guide positioner 810, the tap tool 960, the inserter tool 1600, etc.).

In some embodiments of the targeting guide assembly 800, the handle 850 and adjustable insert retainer 860 may be inserted over the distal end 872 of the positioner shaft 813 (e.g., see FIG. 45) to couple with the targeting guide positioner 810. Once the targeting guide insert 840 and the targeting guide positioner 810 have been attached to the targeting guide base 820, the adjustable insert retainer 860 may then be slid along the positioner shaft 813 distally 702 (e.g., see FIG. 46) to internally couple the adjustable insert retainer 860 to the one or more shaft grooves 815 that are formed in the positioner shaft 813 and retain the targeting guide insert 840 to the targeting guide assembly 800. Likewise, the adjustable insert retainer 860 may be slid along the positioner shaft 813 proximally 701 (e.g., see FIG. 48A) to decouple the adjustable insert retainer 860 from the one or more shaft grooves 815 and release the targeting guide insert 840 from the targeting guide assembly 800.

Once the guide pin 730 has been properly placed within the head 720 of the femur 700, the targeting guide assembly 800 may be disassembled and removed from the femur 700, as previously described herein, by sliding the targeting guide insert 840 proximally off the guide pin 730, removing the one or more placement pins 830, and moving the targeting guide base 820 superiorly to release the guide pin 730 inferiorly through the removal slot 826.

FIG. 49 illustrates a side view of a guide pin depth gauge 990 adjacent the femur 700 in order to measure a depth of the guide pin 730 inside the femur 700. The guide pin depth gauge 990 may include one or more markings 991 configured to indicate a depth of the guide pin 730 inside the femur 700, which may then be utilized to determine a selected length for the femoral fastener 300.

Once the depth of the guide pin 730 has been determined, a reamer tool 1100 and/or a tissue shield 1000 may be placed adjacent the femur 700 (e.g., see FIG. 50) in order to ream a bone tunnel in the femur 700 that may be shaped to receive the barrel 405 of the femoral support member 400 therein.

In some embodiments, a distal end 1010 of the tissue shield 1000 may be notched to conform to the lateral surface 705 of the femur 700.

In some embodiments, the reamer tool 1100 may also include a flared portion 1110 that may be configured to provide a countersink in the femur 700 to receive the base 440 of the barrel 405 therein (see FIGS. 36D and 37D).

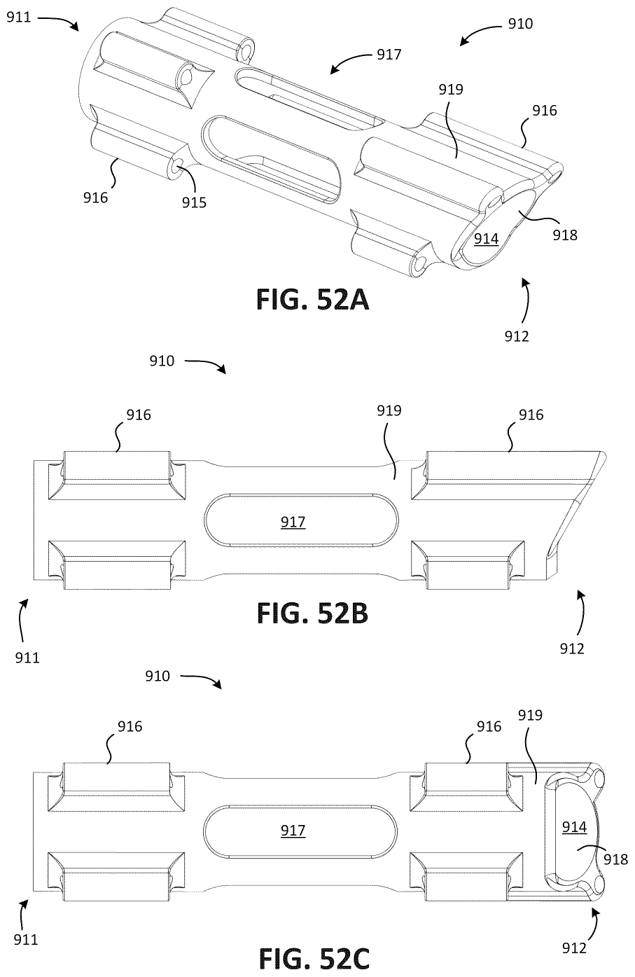

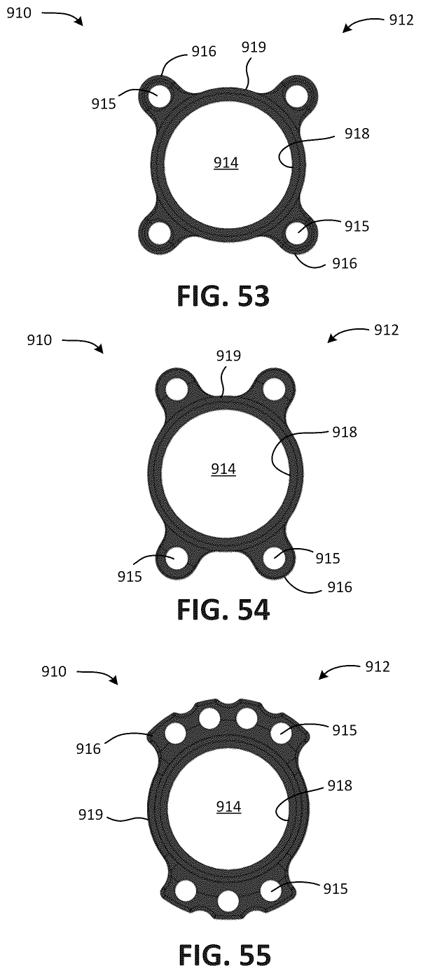

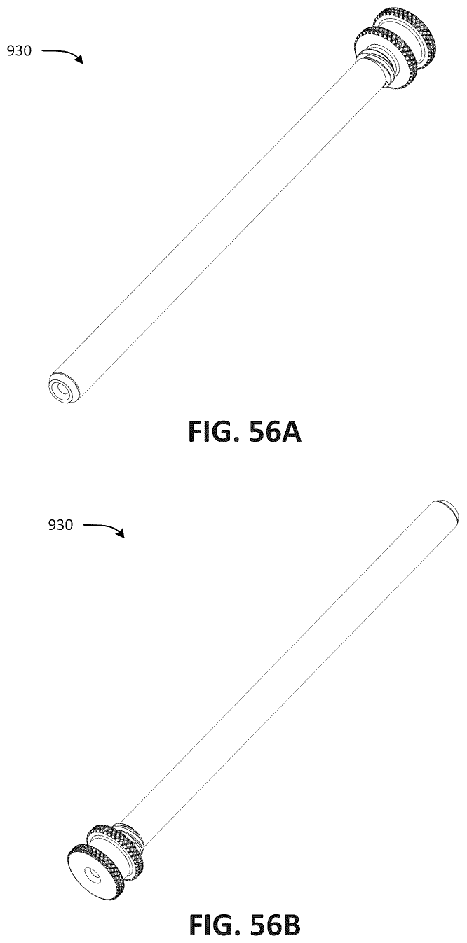

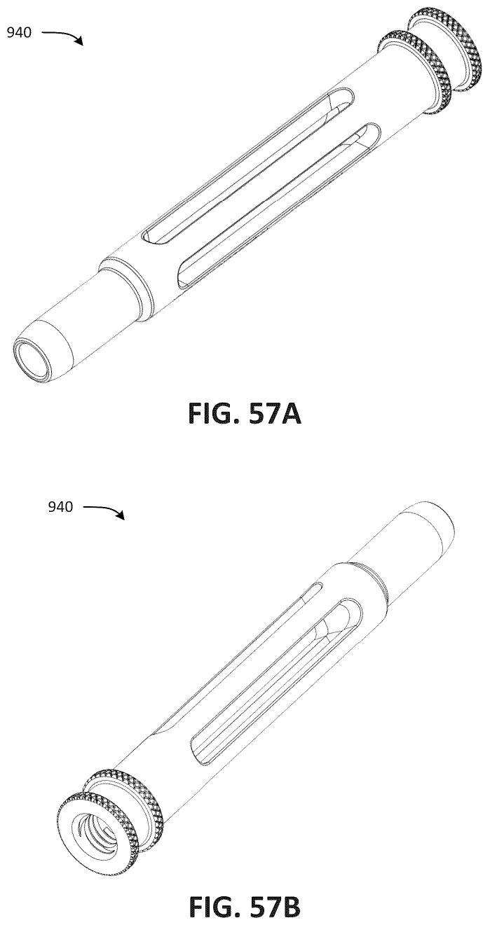

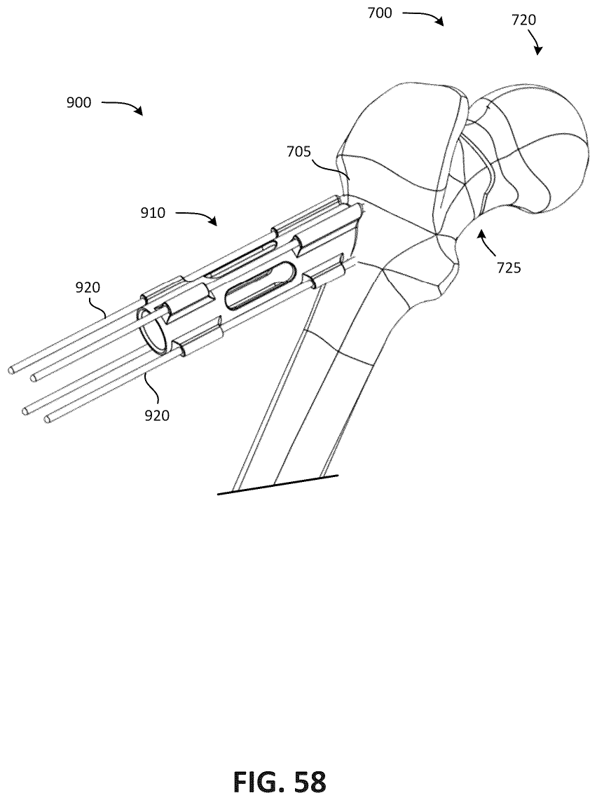

FIGS. 51A-67 illustrate various views of an anti-rotation assembly 900 and its method of use for installing the guide pin 730 into the neck 710 and head 720 of the femur 700. Specifically, FIGS. 51A-51B show an anti-rotation sleeve 910, according to an embodiment of the present disclosure; FIGS. 52A-52C show an anti-rotation sleeve 910, according to another embodiment of the present disclosure; FIGS. 53-55 show distal ends of anti-rotation sleeves, according to embodiments of the present disclosure; FIGS. 56A-57B show a guide wire centering sleeve and a tool centering sleeve, according to embodiments of the present disclosure; and FIGS. 58-67 show the anti-rotation assembly 900 being assembled onto the femur 700 and utilized to perform one or more operations in placing the guide pin 730 in the neck 710 and head 720 of the femur 700.

With reference to FIGS. 51A-55, the anti-rotation sleeve 910 may generally include a proximal end 911, a distal end 912, an anti-rotation sleeve passageway 914 extending between the proximal end 911 and the distal end 912 that defines an inner surface 918 therein, and at least one anti-rotation pin hole 915 disposed about the inner surface 918 of the anti-rotation sleeve 910 that may be configured to receive at least one anti-rotation placement pin 920 therethrough to couple the anti-rotation sleeve 910 to the femur 700.

In some embodiments, the anti-rotation sleeve passageway 914 may be cylindrical in shape. However, it will be understood that the anti-rotation sleeve passageway 914 can comprise any shape without departing form the spirit or scope of the present disclosure (e.g., square, rectangular, triangular, hexagonal, octagonal, oval, etc.).

In some embodiments, the anti-rotation sleeve 910 may also include at least one anti-rotation sleeve window 917 formed through a wall of the anti-rotation sleeve 910. However, it will be understood that the anti-rotation sleeve 910 may not include the at least one anti-rotation sleeve windows 917, in at least some embodiments.

In some embodiments, the distal end 912 of the anti-rotation sleeve 910 may be straight (e.g., see FIGS. 51A-51B).

In some embodiments, the distal end 912 of the anti-rotation sleeve 910 may be notched or angled (e.g., at about 130 degrees, etc.) to better conform to the lateral surface 705 of the femur 700 (e.g., see FIGS. 52A-52C).

In some embodiments, the anti-rotation sleeve 910 may also include at least one anti-rotation pin hole projection 916 that may project away from an outer surface 919 of the anti-rotation sleeve 910. In these embodiments, the at least one anti-rotation pin hole 915 may be formed through the at least one anti-rotation pin hole projection 916 that projects away from the outer surface 919 of the anti-rotation sleeve 910. However, it will be understood that in at least some embodiments the anti-rotation sleeve 910 may not include the at least one anti-rotation pin hole projection 916 (e.g., the at least one anti-rotation pin hole 915 may be formed within the body of the anti-rotation sleeve 910 intermediate the inner surface 918 and the outer surface 919).

In some embodiments, the at least one anti-rotation placement pin 920 may comprise a plurality of anti-rotation placement pins, and the at least one anti-rotation pin hole 915 may comprise a plurality of anti-rotation pin holes disposed about the inner surface 918 of the anti-rotation sleeve 910 and configured to receive the plurality of anti-rotation placement pins therethrough. The plurality of anti-rotation pin holes may be arranged about the inner surface 918 of the anti-rotation sleeve 910 in any number and/or in any spaced relationship with each other (e.g., see the configuration shown in FIGS. 53-55, as at least some non-limiting examples).

In some embodiments, the anti-rotation assembly 900 may include the anti-rotation sleeve 910 and the at least one anti-rotation placement pin 920.

In some embodiments, the anti-rotation assembly 900 may also include at least one centering sleeve that is receivable within the anti-rotation sleeve passageway 914. In some embodiments, the at least one centering sleeve may comprise at least one of: a tool centering sleeve 940, a guide wire centering sleeve 930, and a femoral fastener centering sleeve 950.

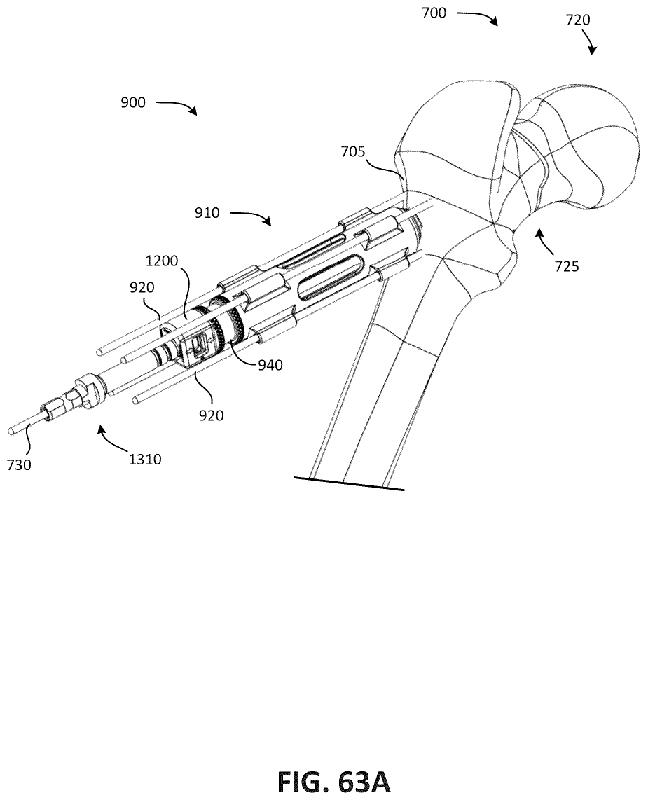

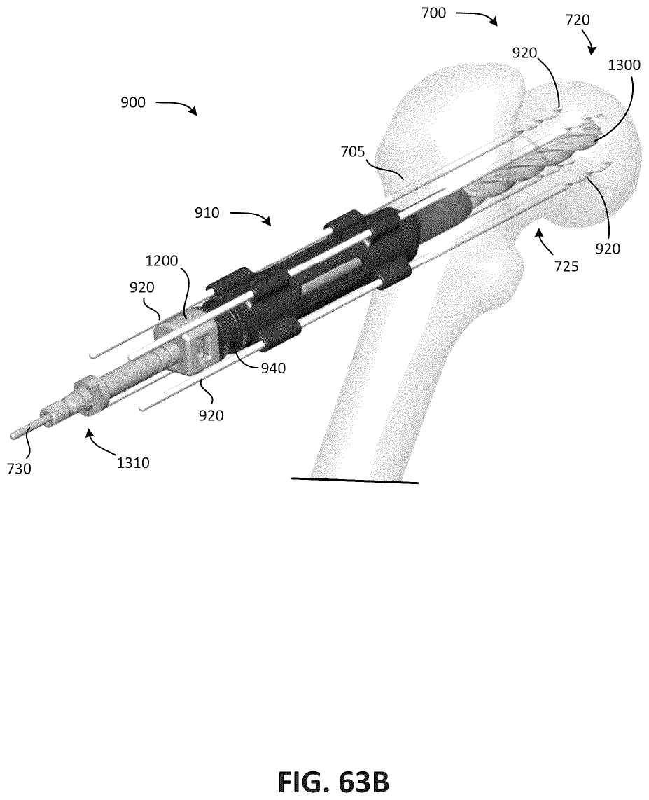

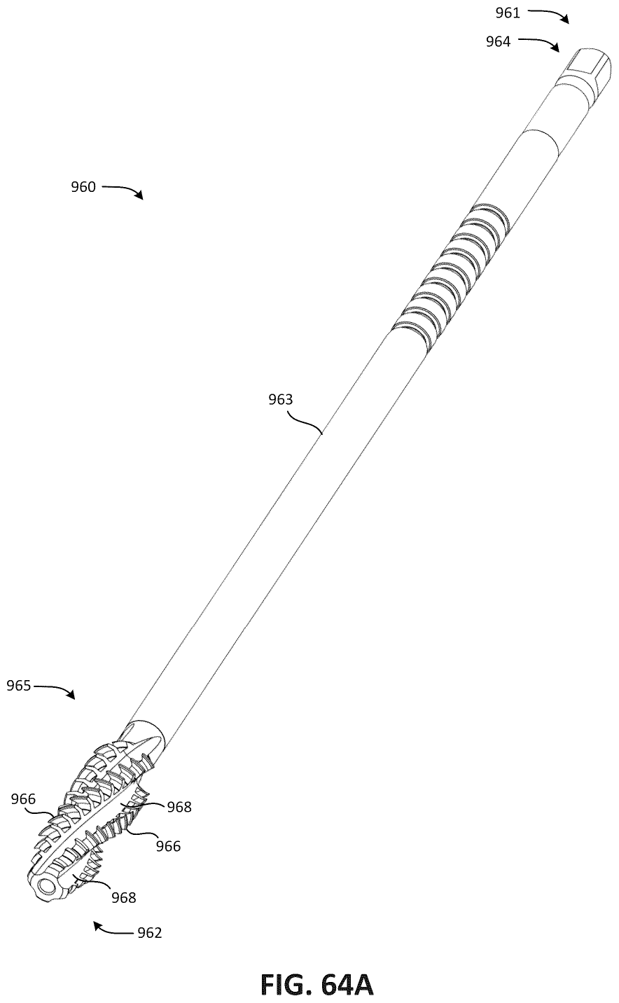

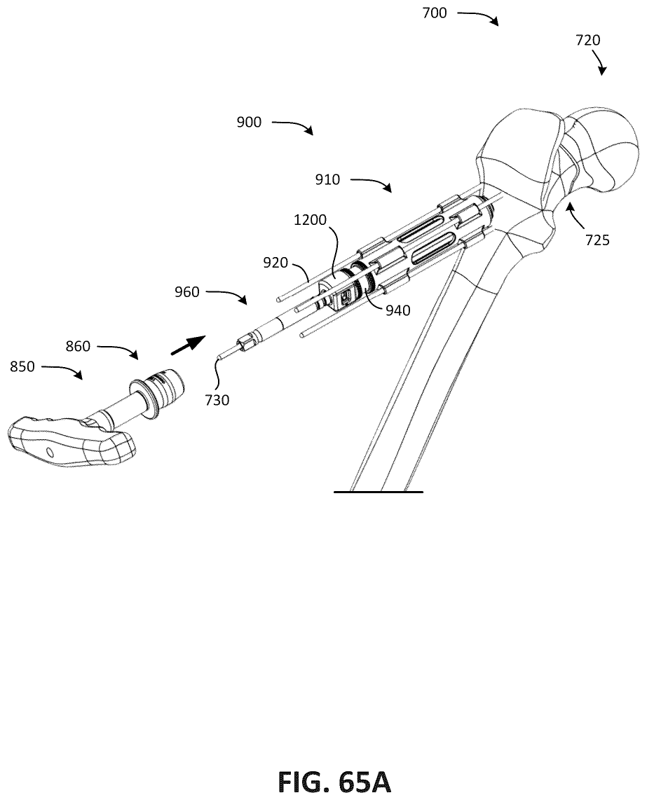

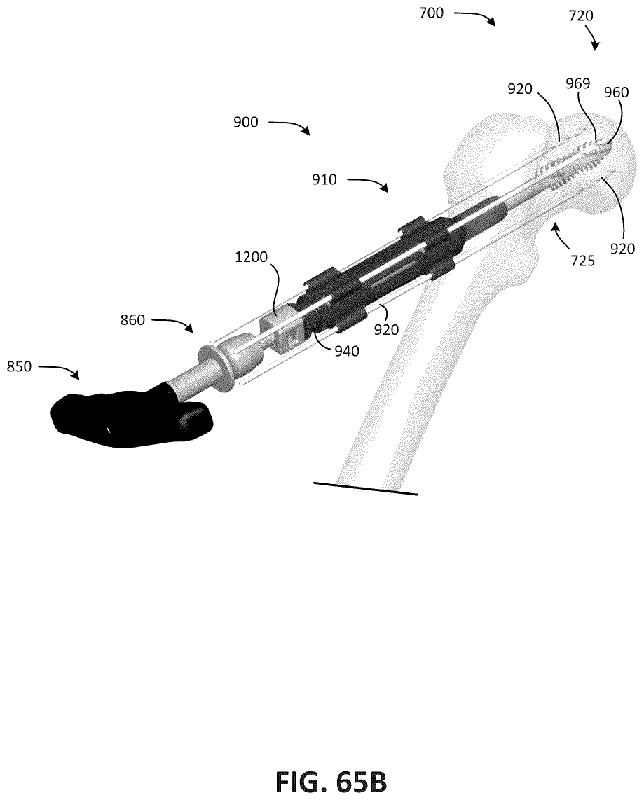

In some embodiments, an inner diameter of the anti-rotation sleeve passageway 914 may conform to an outer diameter of the tool centering sleeve 940 in order to center at least one rotatable tool (e.g., a drill tool 1310, a tap tool 960, an inserter tool 1600, a reamer tool 1100, etc., see FIGS. 63A, 63B, 65A. 65B, and 67) that is placed therethrough within the tool centering sleeve 940 and within the anti-rotation sleeve passageway 914.

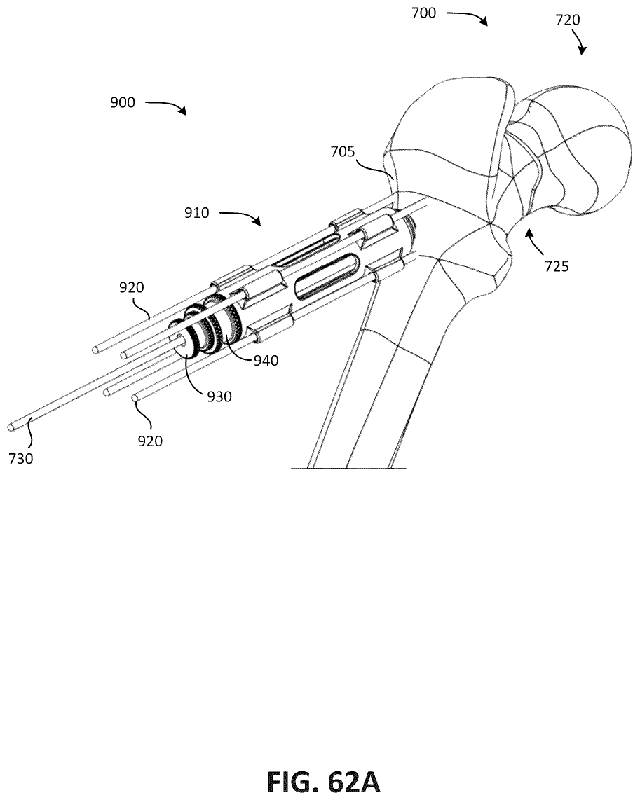

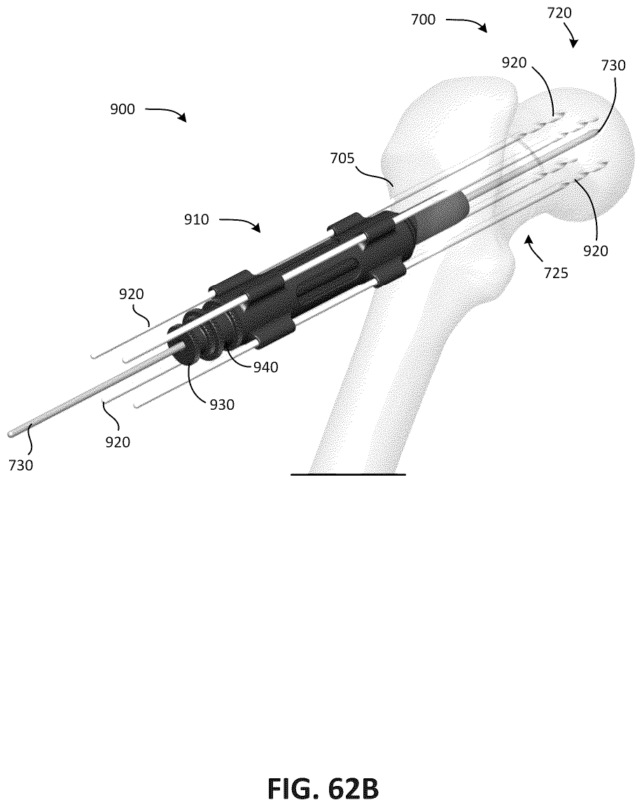

In some embodiments, an inner diameter of the tool centering sleeve 940 may conform to an outer diameter of the guide wire centering sleeve 930 in order to center the guide pin 730 placed within the guide wire centering sleeve 930, the tool centering sleeve 940, and the anti-rotation sleeve passageway 914 (e.g., see FIGS. 62A and 62B). In these embodiments, the guide wire centering sleeve 930 may utilize the orientation of the guide pin 730 to center the anti-rotation assembly 900 thereabout during assembly of the anti-rotation assembly 900 onto the femur 700.

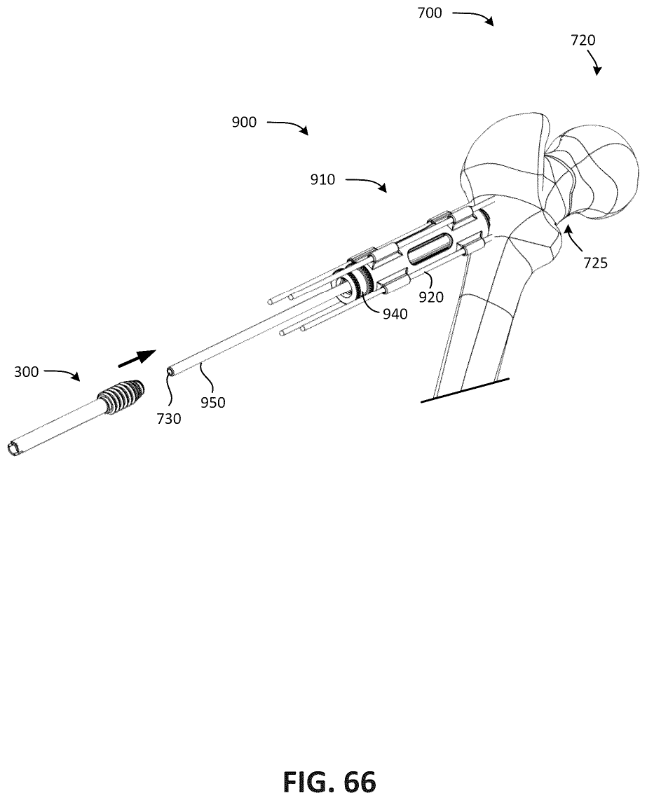

In some embodiments, an inner diameter of the femoral fastener centering sleeve 950 may conform to an outer diameter of the guide pin 730, and an outer diameter of the femoral fastener centering sleeve 950 may conform to an inner diameter of the tool centering sleeve 940, in order to center the femoral fastener 300 within the tool centering sleeve 940 and within the anti-rotation sleeve passageway 914 (e.g., see FIG. 66).

The anti-rotation assembly 900 may be utilized during at least the reaming, drilling, tapping, and femoral fastener 300 insertion surgical steps. Multiple anti-rotation placement pins may be placed around the inscribed circumference of the neck 710 of the femur 700 to cross the fracture 725 and hold the neck 710 and head 720 of the femur 700 in their reduced state to resist rotational torque forces placed upon the construct (e.g., via manual or power rotational instrumentations, such as drills, reamers, taps, femoral fastener inserters, etc.). The anti-rotation placement pins may be placed about the guide pin 730 (e.g., cylindrically, etc.) so as not to interfere with subsequent drilling, tapping, and femoral fastener insertion surgical steps.

With reference to FIGS. 58-62B, the anti-rotation assembly 900 may be coupled to the femur 700 by positioning the distal end 912 of the anti-rotation sleeve 910 adjacent the lateral surface 705 of the femur 700, and then inserting the at least one anti-rotation placement pin 920 through the at least one anti-rotation pin hole 915 and into the neck 710 and head 720 of the femur 700 in order to stabilize the fracture 725. In this manner, the anti-rotation sleeve 910 rigidly couples the neck 710 and the head 720 of the femur 700 together across the fracture 725 with the anti-rotation sleeve passageway 914 of the anti-rotation sleeve 910 configured to receive at least one rotatable tool therethrough that imparts at least one torque force on the head 720 of the femur 700 relative to the neck 710 of the femur 700 across the fracture 725. However, because the anti-rotation assembly 900 is configured to resist the at least one torque force that is imparted on the head 720 of the femur 700 by the rotatable tool, the anti-rotation assembly 900 maintains stabilization of the fracture 725 and prevents the head 720 of the femur 700 from spinning/rotating as the tool rotates. Thus, the anti-rotation assembly 900 acts to limit rotational torque forces and reduce the chance of rotational displacement of the head 720 during surgical procedures, such as femoral neck fracture and intertrochanteric fracture open reduction internal fixation (ORIF) procedures.

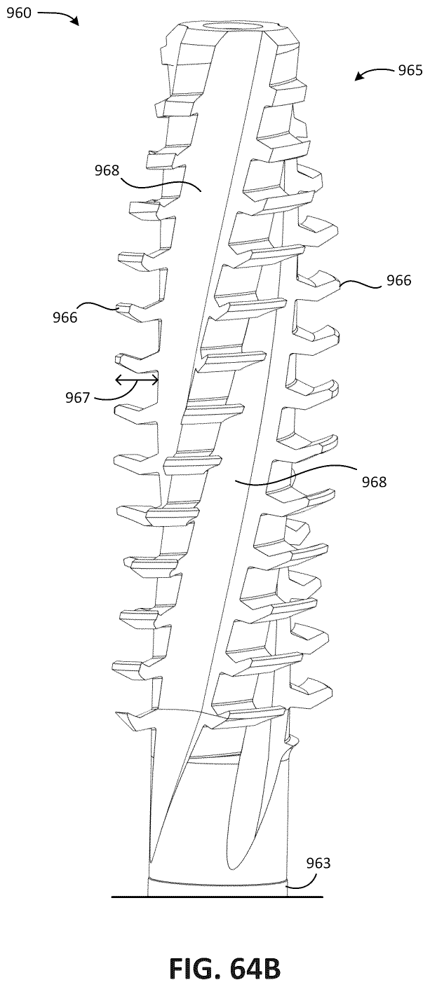

With reference to FIGS. 64A-65B, a tap tool 960 is shown in accordance with an example tap tool of the present disclosure. The tap tool 960 may generally include a proximal end 961, a distal end 962, a tap tool shaft 963, a tap tool torque connection feature 964, a tap tool head 965, cutting teeth 966 disposed about the tap tool head 965 having a height 967, and at least one cutting flute 968 formed within the tap tool head 965.

In some embodiments, the tap tool 960 may comprise a set of tap tools comprising at least a first tap tool with first cutting teeth having a first height, and a second tap tool with second cutting teeth having a second height. The first and second tap tools may generally include the same shape, features, and characteristics of tap tool 960 shown in FIGS. 64A-64B, with the only difference being the relative heights of the cutting teeth between the first and second tap tools.

In some embodiments, the first height of the first cutting teeth may be less than the second height of the second cutting teeth.

In some embodiments, the first height of the first cutting teeth may be about half of the second height of the second cutting teeth.

In some embodiments, the first tap tool may be configured to cut a first tapped bone thread 969 in the head 720 of the femur 700 at the first height of the first cutting teeth, thereby imparting a first torque force on the head 720 relative to the neck 710 across the fracture 725 as the first tap tool rotates. The second tap tool may then be configured to follow the first tapped bone thread 969 and cut the remaining height of the first tapped bone thread to the second height of the second cutting teeth, thereby imparting a second torque force on the head 720 relative to the neck across the fracture 725 as the second tap tool rotates. In this manner, the first and second torque forces imparted on the head 720 will be reduced by cutting the first tapped bone thread 969 in stages, or in succession, to reduce the torque forces applied to the head 720 and help maintain stabilization of the fracture 725.

In some embodiments, the tap tool 960 may also comprise a plurality of tap tools having a plurality of cutting teeth heights intermediate the first height and the second height that are configured to further reduce torque forces applied to the head 720 by successively cutting the first tapped bone thread into the head 720 with progressively taller cutting teeth heights in order to maintain stabilization of the fracture 725.

In some embodiments, the plurality of cutting teeth heights are configured to increase in a linear fashion between the first height and the second height.

In some embodiments, the plurality of cutting teeth heights are configured to increase in a non-linear fashion between the first height and the second height. However, it will be understood that any number of cutting teeth heights arranged in any fashion may be utilized.

In some embodiments, at least one of: the first tap tool may comprise at least one first cutting flute configured to move bone chips in a proximal direction along the first tap tool, and the second tap tool may comprise at least one second cutting flute configured to move the bone chips in the proximal direction along the second tap tool.

In some embodiments, at least one of: the first tap tool may comprise a first tap tool torque connection feature configured to receive torque forces to rotate the first tap tool, and the second tap tool may comprise a second tap tool torque connection feature configured to receive torque forces to rotate the second tap tool.

With reference to FIG. 68, once the femoral fastener 300 has been implanted within the neck 710 and head 720 of the femur 700, a tamp or impactor 1700 may be slid over the guide pin 730 and a mallet 1800 may be utilized to seat the femoral support member 400 against the lateral surface 705 of the femur 700 in preparation for final attachment to the femur 700.

FIGS. 69 and 70 show the femoral fixation assembly 600 completely installed on the femur 700 with one or more support fasteners 2800 placed through the neck 710 and head 720 of the femur 700 about the femoral fastener 300, and one or more bone plate fasteners 2500 placed within the shaft of the femur 700 to secure the femoral support member 400 thereto, similar to the process previously described with reference to FIGS. 24-31.

Any of the fasteners described herein may be configured for removal and replacement during a revision procedure by simply unscrewing and removing the fastener from the bone/tissue in which the fastener resides. Moreover, the fasteners described herein may advantageously be removed from bone without removing any appreciable amount of bone during the removal process to preserve the bone. In this manner, implants may be mechanically integrated with the bone, while not being cemented to the bone or integrated via bony ingrowth, in order to provide an instant and removable connection between an implant and a bone. Accordingly, revision procedures utilizing the fasteners described herein can result in less trauma to the bone and improved patient outcomes.

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein.

The phrases “connected to”, “coupled to”, “engaged with”, and “in communication with” may refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, components that are removably and/or non-removably coupled with each other, components that are functionally coupled to each other through one or more intermediary components, etc. The term “abutting” refers to items that may be in direct physical contact with each other, although the items may not necessarily be attached together. The phrase “fluid communication” refers to two or more features that are connected such that a fluid within one feature is able to pass into another feature. As defined herein the term “substantially” means within +/−20% of a target value, measurement, or desired characteristic.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, and methods disclosed herein.

What is claimed is:

1. A surgical kit for implanting a femoral fastener in a femoral bone, the surgical kit comprising:
a femoral fastener comprising:
a shaft comprising:
a longitudinal axis;
a proximal end;
a distal end; and
a longitudinal passageway formed through the shaft; and
a helical thread disposed about the shaft between a first location and a second location along the shaft, the helical thread comprising at least one undercut surface;
wherein, when the femoral fastener is implanted within a neck and a head of a femoral bone:
at least a portion of the helical thread resides within the head of the femoral bone;
the at least one undercut surface of the helical thread is angled toward one of the proximal end and the distal end of the femoral fastener; and
the at least one undercut surface of the helical thread is configured to transmit at least one force from the head of the femoral bone to the neck of the femoral bone; and
a targeting guide assembly comprising:
a targeting guide base comprising:
a superior end;
an inferior end;
a bone-facing surface;
an opposing surface, opposite the bone-facing surface;
a locating slot formed through the targeting guide base between the bone-facing surface and the opposing surface; and
a removal slot formed through the targeting guide base between the bone-facing surface and the opposing surface, the removal slot extending from the inferior end of the targeting guide base to the locating slot; and
a targeting guide insert comprising:
a proximal end;
a distal end;
at least one insert passageway formed through the targeting guide insert between the proximal end and the distal end of the targeting guide insert; and
an insert base connection feature at the distal end of the targeting guide insert;
wherein:
the bone-facing surface of the targeting guide base is configured to engage a lateral surface of the femoral bone;
the insert base connection feature is configured to removably couple the targeting guide insert to the targeting guide base at the locating slot;
the at least one insert passageway of the targeting guide insert is configured to orient a guide pin placed therethrough into the neck and head of the femoral bone to guide placement of the femoral fastener therein;
once the guide pin has been placed into the neck and the head of the femoral bone, the targeting guide insert is configured to detach from the targeting guide base and slidingly disengage from the guide pin in a proximal direction; and
the targeting guide base is movable in a superior direction to release the guide pin inferiorly through the removal slot that extends from the locating slot to the inferior end of the targeting guide base.

2. The surgical kit of claim 1, wherein the at least one insert passageway comprises at least one of:
a cylindrical shape;
a conical shape;
an oval shape;
a flared shape; and
a tapered shape.

3. The surgical kit of claim 1, wherein:
the at least one insert passageway comprises a plurality of insert passageways formed through the targeting guide insert.

4. The surgical kit of claim 1, wherein:
the insert base connection feature comprises an insert projection configured to be at least partially received within the locating slot of the targeting guide base.

5. The surgical kit of claim 1, further comprising:
a targeting guide positioner comprising:
a positioner shaft;
a positioner first arm projecting from a distal end of the positioner shaft;
a positioner second arm projecting from the distal end of the positioner shaft, opposite the positioner first arm;
at least one positioner base connection feature configured to couple the targeting guide positioner to the targeting guide base; and
at least one positioner insert connection feature configured to couple the targeting guide positioner to the targeting guide insert.

6. The surgical kit of claim 1, wherein the targeting guide base further comprises:
one or more placement pin holes configured to receive one or more placement pins therethrough to couple the targeting guide base to the femoral bone.

7. The surgical kit of claim 1, wherein the targeting guide base further comprises:
one or more bone fixation members configured to stabilize the targeting guide base with respect to the femoral bone.

8. A surgical kit for implanting a femoral fastener in a femoral bone, the surgical kit comprising:
a femoral fastener comprising:
a shaft comprising:
a longitudinal axis;
a proximal end;
a distal end; and
a longitudinal passageway formed through the shaft; and
a helical thread disposed about the shaft between a first location and a second location along the shaft, the helical thread comprising at least one undercut surface;
wherein, when the femoral fastener is implanted within a neck and a head of a femoral bone:
at least a portion of the helical thread resides within the head of the femoral bone;
the at least one undercut surface of the helical thread is angled toward one of the proximal end and the distal end of the femoral fastener; and
the at least one undercut surface of the helical thread is configured to transmit at least one force from the head of the femoral bone to the neck of the femoral bone; and
an anti-rotation assembly comprising:
at least one anti-rotation placement pin; and
an anti-rotation sleeve comprising:
a proximal end;
a distal end;
an anti-rotation sleeve passageway extending between the proximal end and the distal end of the anti-rotation sleeve that defines an inner surface therein; and
at least one anti-rotation pin hole disposed about the inner surface of the anti-rotation sleeve that is configured to receive the at least one anti-rotation placement pin therethrough;
wherein, when the anti-rotation assembly is coupled to the femoral bone:
the distal end of the anti-rotation sleeve is positioned adjacent a lateral surface of the femoral bone;
the at least one anti-rotation placement pin is inserted through the at least one anti-rotation pin hole and into the neck and the head of the femoral bone to stabilize a bone fracture formed between the neck and the head of the femoral bone;
the anti-rotation sleeve passageway is configured to receive at least one rotatable tool therethrough that imparts at least one torque force on the head of the femoral bone relative to the neck of the femoral bone across the bone fracture; and
the anti-rotation assembly is configured to resist the at least one torque force imparted on the head to stabilize the bone fracture as the at least one rotatable tool rotates.

9. The surgical kit of claim 8, wherein:
the at least one anti-rotation placement pin comprises a plurality of anti-rotation placement pins; and
the at least one anti-rotation pin hole comprises a plurality of anti-rotation pin holes disposed about the inner surface of the anti-rotation sleeve and configured to receive the plurality of anti-rotation placement pins therethrough.

10. The surgical kit of claim 8, wherein the distal end of the anti-rotation sleeve is angled.

11. The surgical kit of claim 8, wherein the distal end of the anti-rotation sleeve is straight.

12. The surgical kit of claim 8, wherein:
the anti-rotation sleeve further comprises at least one anti-rotation pin hole projection; and
the at least one anti-rotation pin hole is formed through the at least one anti-rotation pin hole projection.

13. The surgical kit of claim 8, further comprising the at least one rotatable tool, wherein the at least one rotatable tool comprises at least one of:
a reamer tool;
a drill tool;
a tap tool; and
an inserter tool.

14. The surgical kit of claim 8, further comprising at least one centering sleeve receivable within the anti-rotation sleeve passageway, wherein the at least one centering sleeve comprises at least one of:
a tool centering sleeve;
a guide wire centering sleeve; and
a femoral fastener centering sleeve.

15. A surgical kit for implanting a femoral fastener in a femoral bone, the surgical kit comprising:
a femoral fastener comprising:
a shaft comprising:
a longitudinal axis;
a proximal end;
a distal end; and
a longitudinal passageway formed through the shaft; and
a helical thread disposed about the shaft between a first location and a second location along the shaft, the helical thread comprising at least one undercut surface;
wherein, when the femoral fastener is implanted within a neck and a head of a femoral bone:
at least a portion of the helical thread resides within the head of the femoral bone;
the at least one undercut surface of the helical thread is angled toward one of the proximal end and the distal end of the femoral fastener; and
the at least one undercut surface of the helical thread is configured to transmit at least one force from the head of the femoral bone to the neck of the femoral bone across a bone fracture that is formed therebetween;
a first tap tool comprising:
first cutting teeth having a first height; and
a second tap tool comprising:
second cutting teeth having a second height;
wherein:
the first height of the first cutting teeth is less than the second height of the second cutting teeth;
the first tap tool is configured to cut a first tapped bone thread in the head of the femoral bone at the first height of the first cutting teeth, thereby imparting a first torque force on the head relative to the neck across the bone fracture as the first tap tool rotates; and
the second tap tool is configured to follow the first tapped bone thread and cut a remaining height of the first tapped bone thread at the second height of the second cutting teeth, thereby imparting a second torque force on the head relative to the neck across the bone fracture as the second tap tool rotates;
wherein, the second torque force imparted on the head is reduced by cutting the first tapped bone thread in the head at the first height before cutting the remaining height of the first tapped bone thread at the second height to reduce torque forces applied to the head and maintain stabilization of the bone fracture.

16. The surgical kit of claim 15, further comprising:
a plurality of tap tools having a plurality of cutting teeth heights intermediate the first height and the second height that are configured to reduce torque forces applied to the head by successively cutting the first tapped bone thread into the head to maintain the stabilization of the bone fracture.

17. The surgical kit of claim 16, wherein the plurality of cutting teeth heights are configured to increase in a linear fashion between the first height and the second height.

18. The surgical kit of claim 16, wherein the plurality of cutting teeth heights are configured to increase in a non-linear fashion between the first height and the second height.

19. The surgical kit of claim 15, wherein at least one of:
the first tap tool comprises at least one first cutting flute configured to move bone chips in a proximal direction along the first tap tool; and
the second tap tool comprises at least one second cutting flute configured to move the bone chips in the proximal direction along the second tap tool.

20. The surgical kit of claim 15, wherein at least one of:
the first tap tool comprises a first tap tool torque connection feature configured to receive torque forces to rotate the first tap tool; and
the second tap tool comprises a second tap tool torque connection feature configured to receive torque forces to rotate the second tap tool.
